(19) United States
(12) Patent Application Publication
(10) Pub. No.: US 2010/0036280 A1
(43) Pub. Date: Feb. 11, 2010

(54) METHOD OF UTILISING MEASUREMENTS OF THRESHOLD OF PAIN

(75) Inventors: Soren Ballegaard, Hellerup (DK); Gisli Magnusson, Hellerup (DK)

Correspondence Address: BIRCH STEWART KOLASCH & BIRCH, PO BOX 747, FALLS CHURCH, VA, 22040-0747, US

(73) Assignee: ULL METER A/S, Hellerup (DK)

(21) Appl. No.: 12/440,438

(22) PCT Filed: Sep. 10, 2007
(86) PCT No.: PCT/EP2007/059471

§ 371 Date: Oct. 6, 2009

Related U.S. Application Data
(60) Provisional application No. 60/842,984, filed on Sep. 8, 2006.
(60) Provisional application No. 60/829,068, filed on Oct. 11, 2006.
(60) Provisional application No. 60/924,986, filed on Jun. 7, 2007.

(30) Foreign Application Priority Data
Sep. 8, 2006 (DK) PA 2006 01159
Oct. 11, 2006 (DK) PA 2006 01321
Feb. 2, 2007 (DK) PA 2007 00190
Jun. 6, 2007 (EP) 07109765.3

(51) Int. Cl. A61B 5/00

(52) U.S. Cl. 600/552; 600/557; 600/554; 600/555

(57) ABSTRACT

A method of determining the sympathetic tone and/or level of stress and/or level or warning system sensitivity includes the steps of: measuring an applied stimulation at a threshold value of the stimulation in one or more sympathetic tone-neutral points and measuring an applied stimulation at the same threshold value in one or more sympathetic tone-dependent points. The invention further relates to a system for applying and measuring a stimulation, and the use of a system for applying and measuring a stimulation for determining the sympathetic tone including the steps of: measuring an applied stimulation at a threshold value of the stimulation at one or more sympathetic tone-neutral points and measuring an applied stimulation at the same threshold value of the stimulation at one or more sympathetic tone-dependent points.

### TECHNICAL FIELD

The invention relates to a method of here-and-now determination of the sympathetic tone/the WSS/the DRS and a system for measurement thereof. As the method provides the person with a here-and-now determination of the sympathetic tone possibly in combination with a track record of measurements it is applicable for determining healthy individuals' level of potential for performing optimally both physically and mentally. The invention further relates to use of the system according to the invention for determining the sympathetic tone and use of measuring of the nociception for determining the sympathetic tone.

### BACKGROUND ART

In mammals the nervous system is functionally divided into a somatic nervous system and an autonomic nervous system. The autonomic nervous system functions automatically and reflectory. The autonomic nervous system can further be divided into the counteracting sympathetic and parasympathetic systems. The sympathetic and parasympathetic nerves have opposite effects.

The sympathetic nervous system mobilizes the resources in the organism in a so-called “stress response” such that an immediate dangerous situation/a challenge can be handled in the optimum manner. When the stress response has caused the demanded changes in the function of the body, a new state of the body is established, which is called the “stress phase”. In this phase, mentally the person thinks faster and more clearly at the same time as sharpening the ability to focus his/her thoughts. For supporting this purpose, irrelevant sense impressions are effectively impeded. Physically the body responds by lowering the response time, increasing the muscle strength, sharpening the senses, and optimizing the coordination between thought and motor skills.

In conclusion, the above entails that the “stress phase” is a positive physiological phenomenon, when it manifests itself in the right amount and in the right balance with the necessary recovery, which as described below is effected when the parasympathetic nervous system dominates.

The parasympathetic nervous system mediates the biological processes, which restores and builds up the organism's resources and thereby ensures that the necessary resources are available when they are to be mobilized in an acute/transient stress situation.

Physiologically, simulation via the sympathetic nerves increases the pulse and the blood pressure and inhibits the secretion formation in the glands, etc., whereas the parasympathetic nerves inter alia lower the heart rate and the blood pressure to a physiological resting state level and stimulate glands to secrete. During stress the sympathetic nervous system is activated.

Stress can be pleasant, but can also be unpleasant and sometimes dangerous to life. It is essential to distinguish between two forms of stress: transient and persistent. Transient stress (=acute stress) is the physiological state of preparedness, a state which is automatically induced in the body through neural/hormonal signals from the brain when a threat is perceived. Thus, it serves as a defence mechanism. The level of stress depends on the balance between the individual expectancies of the outcome of the stimulus and the resources available. When the challenge/threat is over, homeostasis is re-established. If the person has the necessary resources available, the situation may be perceived in a positive manner. If the situation represents a state in which the strain exceeds the resources of the body, the situation will most likely be perceived negatively. If this latter situation situation continues, the resssources of the organism may become taxed and the performance of the body impaired. This state is called persistent stress or chronic stress, and is a dysfunction of the neural/hormonal processes of the brain due to a prolonged exposure to the neural processes/hormones involved in transient stress, and with insufficient restitution in between. It can be harmful to our health.

In its mildest form persistent or chronic stress manifests itself as moderate bodily symptoms such as muscle tension, fatigue or headache. In a more severe degree of stress additional symptoms are experienced in form of for instance memory problems, lack of concentration and distress from the internal organs (e.g. palpitations, stomach ache, decreased libido). In an even more serious stress state, social ability is also impaired, e.g. reduced tolerance, irritability and uncontrolled bursts of anger. In the latter case, untreated chronic stress may lead to illness whereby the working capacity is lost for a period of time.

The body's reaction to the above mentioned strain/perception of threat is controlled by the hypothalamic-pituitary-adrenal system which activates the release of steroid hormones (glycocorticoids) including cortisol. Additionally, other hormones are released among others catecholamines including dopamine, noradrenaline and adrenaline. As a result, a set of physiological reactions are created which in combination is called the stress response. Substantially all the systems of the body are affected including the brain, the cardiovascular system, the immune system, the respiratory system and the digestive system.

When the physical and mental dangers/challenges/strains have passed, the body's response thereto is inactivated and the recovery phase begins.

The stress response is not activated by purely physical or psychological threats, but also by our thoughts. A number of everyday-life situations inter alia rush for time, worries, personal relationship problems and financial worries, may activate the stress response without the person being threatened for life. The accumulated effect of these minor but daily strains may lead to persistent/chronic stress.

As a part of avoiding that the stress condition develops and thus leads to negative implications, the determination of a person's acute or accumulated stress level is vital to allow for cognitive processing and proper actions to be initiated which can reduce or completely remove the strains causing the stress and/or the person's readiness to handle these strains can be increased such that the negative stress-related consequences—both personal and social—may be averted and/or prevented. Stress cannot per se be considered an illness, but accumulated stress can make a person more susceptible to impacts which may develop into an illness.

A number of methods are known for determining the sympathetic tone (the activity of the sympathetic nervous system) as a measure of a person's stress level including measuring of cortisol in saliva, measuring of catecholamines (adrenaline and/or noradrenaline) and cortisol in serum as well as measuring of catecholamines in urine (Ekman R. and Lindstedt. G.: “Molekyler på liv og død” (molecules in life and death), in Ekman R. and Arnetz B. (red) “Stress; Molekylerne, Individen, Organisationen, Samhället” (stress; molecules, the individual, organisation and society), Libers publishing firm, Stockholm 2002, pages 77-89; Hansen A. M., Garde A. H., Christensen J. M., Eller N. H. & Nettestrøm B. “Evaluation of a radioimmunoassay and establishment of a reference interval for salivary cortisol in healthy subjects in Denmark”, Scand J Lab Invest 2003; 63: 303-10.). Measurement of skin temperature (Normell L A, Wallin B G. “Sympathetic skin nerve activity and skin temperature changes in man”. Acta Physiol Scand 1974; 91: 417-26) and sweat secretion are other known method for measuring stress.

The known methods of determining stress and sympathetic tone are encumbered by the drawbacks that either complicated technical analyses involving delays, communication and expenses are required or the methods are not unsusceptible to impacts/influences from the physical environment. Serum determination of for instance cortisol requires a laboratory analysis. Additionally one drawback of such a determination is that a change in the serum concentration of cortisol may rely on other causes than an increased level of stress. The sweat secretion determination is encumbered by the drawback that this determination may be highly unreliable, especially on a hot day where sweat secretion increases regardless of the person's level of stress.

Warning systems and defence reaction mechanisms are essential parts of the survival strategy of living organisms. Special polymodal receptors of the nociceptive system are present to detect tissue-damaging environmental stimuli, providing the organism with the information needed for an optimal response to adverse conditions—such as a reflex response or a withdrawal reaction. The receptors are susceptible to modulation by a variety of exogenous and endogenous substances, including sympathetic input and the response being regulated on a molecular level by Ca2⁺-permeable TRPV channels. Stress is known to generally suppress the pain sensation of these sensors.

A need thus exists for a fast, reliable and inexpensive method of determining the sympathetic tone as a measure of a person's potential to perform optimally both physically and mentally.

### DESCRIPTION OF THE INVENTION

The present inventors have previously found that in vital areas, stress increases pain sensation. It is also found that defense reactions/reflexes are rendered more sensitive by stress, thus leading to an enhancement of the survival potential.

It is now demonstrated that the stress increased pain sensitivity of the chest bone observed in humans (cf. WO 2005/084529 and WO 2006/092146) is correlated with a corresponding enhancement of the non-cognitive noxious withdrawal reflex (NWR), indicating a potential beneficial enhancement of the warning system as well as defense reactions and that the two are linked.

Until now, increased pain sensitivity has been regarded as a local response to tissue damage, as in inflammation, or a general dysfunction as in neurogenic pain; hence increased pain sensitivity is not known to be linked to defense reactions.

The sensitivity was measured by simple and reliable means (as taught in the present assignee's WO 2005/084529 and WO 2006/092146), and was shown to normalize when the stress-inducing influence was over. As simple objective and reliable methods for the diagnosis of stress presently do not exist, it is anticipated that the methodology has the potential for a broad range of practical applications, in cases where professionals or non-professional may benefit from a reliable measure for stress, such as in post-traumatic stress disorders in combat soldiers, heart disease, metabolic syndrome (hypertension, adipositas, depression and diabetes mellitus), and in the healthy part of the population for preventative measures as well as in farming animal welfare.

The present invention thus presents a new understanding of the modulatory potential of biological warning systems and defense reactions with the aim to improve survival potential, including both the afferent sensory and the efferent motor response. It may be emphasized that there is a known functional, structural and molecular background for the observed modulations.

Presently, an increase in sensitivity of defence reactions, as reflected in the simplest efferent motor response, the noxious withdrawal reflex (NWR), has not been suggested as part of a generally improved survival potential for either animals or humans during the perception of a threat. The modulation of NWR or other defense reactions by stress or other general environmental factors has not been a topic for investigation. The NWR has been regarded as an objective dose-dependent response to pain. However, local modulation has been observed in case of injury, protecting the injured site.

The link between sensitivity of the warning system and deference reactions is hence new and suggests a new field of biological research.

As stimuli with a potential threat are processed in the brain in the absence of conscious perception, and the conscious perception of clinical stress symptoms may be suppressed in the case of severe stress, the present findings establishes a new path in the research field of the link between conscious and unconscious perception of threats.

Presently, physiological methods to distinguish an increase in activity of the sympathetic nervous system due to stress from that of physical exercise has not been identified. The present invention provides such a method.

The present invention provides a method of determining the sympathetic tone. It is fast, simple, reliable and inexpensive and can be used as a measure of a person's acute and accumulated level of stress but as mentioned above, it may also be useful in establishing the status of an individual's warning system sensitivity (WSS) as well as defense reaction/reflex sensitivity (DRS).

The invention further provides a system for carrying out the method.

In a first aspect, the invention relates to a method of determining the sympathetic tone and/or the level of stress and/or the status of the warning system sensitivity in a subject, including the steps of: measuring an applied stimulation at a threshold value of the stimulation in one or more sympathetic tone-neutral points and measuring an applied stimulation at the same threshold value in one or more sympathetic tone-dependent points, or measuring an applied stimulation at a threshold value in one or more sympathetic tone-dependent points and optionally comparing said threshold value to a predetermined or pre-established calibration threshold value. In a second aspect, the invention relates to a method of quantitative and/or qualitative determination of the level of WSS/DRS and/or sympathetic tone and/or level of stress in an animal, including a human, said method including:
a) storage of a calibration threshold value and a stimulation threshold value, the calibration threshold value being a quantitative measure of a nociception threshold value in a sympathetic tone-neutral point in or on the animal's body and the stimulation threshold value being a quantitative measure of a nociception threshold value in a sympathetic tone-dependent point in or on the body, and subsequently;
b) calculation of an indication value of WSS and/or DRS and/or sympathetic tone and/or level of stress by comparing the stimulation threshold value with the calibration threshold value, whereby the indication value is a measure of the level of WSS/DRS and/or level of stress and/or sympathetic tone in the subject.

A further aspect relates to a method of quantitative and/or qualitative determination of status of WSS/DRS and/or sympathetic tone and/or level of stress in an animal, including a human, said method including:
a) storage of a calibration threshold value and a stimulation threshold value, the calibration threshold value being a quantitative measure of polymodal sensor cell firing threshold in a sympathetic tone-neutral point in or on the animal's body and the stimulation threshold value being a quantitative measure of polymodal sensor cell firing threshold in a sympathetic tone-dependent point in or on the body, and subsequently;
b) calculation of an indication value of warning system sensitivity by comparing the stimulation threshold value with the calibration threshold value, whereby the indication value of warning system sensitivity and/or sympathetic tone and/or level of stress is a measure of the warning system sensitivity and/or sympathetic tone and/or warning system sensitivity in the animal.

Further aspects of the invention relate to various practical implementations of the inventive methods set forth above, where these implementations aim at optimizing treatments and programmes the efficacy or compliance of which are dependent on stress-level and/or sympathetic tone in the individual subjected to the treatment or programme. For instance, the invention relates to a method for controlling the progress of a patient's therapeutic regimen, wherein the efficacy and/or patient compliance of said regimen is dependent on sympathetic tone and/or stress level and/or WSS and/or DRS in said patient, comprising
i) determining one or more times during the course of the therapeutic regimen the sympathetic tone and/or level of stress and/or status of the WSS and/or DRS in said patient, and
ii) adjusting the therapeutic regimen based on an integrated measure of the patient's benefit from the therapeutic regimen and the determination is step i.

Related to these aspects, is a method of the invention which includes an intervention possibility in the event the level of stress and/or sympathetic tone and/or level of WSS and/or DRS is deemed to high by the above-referenced methods. More precisely, the present invention also relates to a method for prevention of undesired or unproductive stress, the method comprising
a) determination of sympathetic tone and/or the stress level and/or level of WSS and/or DRS in a patient by using the herein-described methods for such determination,
   and if the determination in step a indicates an elevated sympathetic tone and/or level of stress and/or level of WSS and/or DRS, subjecting a sympathetic tone dependent point to a stimulation having a lower intensity than the stimulation threshold value for a period of time.

Also embraced by the present invention is a method of prognosis of a disease in a patient, comprising
1) determining the sympathetic tone and/or level of stress and/or level of WSS and/or DRS in the patient, and subsequently
2) providing a prognosis for the patient with respect to the disease by incorporating in the determination of the prognosis the result of the determination in step 1, a determination in step 1 indicating a low sympathetic tone and/or level of stress and/or level of WSS and/or DRS being indicative of a better prognosis than a determination in step 1 of a higher sympathetic tone and/or level of stress and/or level of WSS and/or DRS.

The invention also relates to a method for determining whether an interview-based evaluation of stress level in a subject provides a true indication of stress, comprising,
a) in parallel to the interview, determining the sympathetic tone and/or level of stress and/or level of WSS and/or DRS in the patient by utilising the determination methods described herein, and
b) ascertaining whether the interview-based evaluation provides a result that correlates positively with the determination in step a, a positive correlation indicating that the interview-based evaluation provides a true indication.

In a third aspect, the invention relates to a system for measuring the sympathetic tone and/or level of stress and/or the status of warning system sensitivity in an animal, including a human being, said system including:
a) memory means for storing a nociception calibration threshold value determined in a sympathetic tone-neutral point on or in the human body and for storing a nociception stimulation threshold value determined in a sympathetic tone-dependent point on or in the human body;
b) an electronic circuit programmed to data process the nociception calibration threshold value and the nociception stimulation threshold value so as to obtain the measurement; and
c) user-operated means for applying a discomfort-evoking stimulus to the animal's body and user-operated storage means adapted to store the nociception stimulation threshold value resulting from a first user operation;
d) user-operated means for applying a discomfort-evoking stimulus to the animal's body and user-operated storage means adapted to store the nociception stimulation threshold value resulting from a second user operation; wherein the discomfort-evoking stimulus involves 1) vibration applied by means of a first vibration base and/or 2) heat applied by means of a first heating base and/or 3) electricity applied by means of a first electricity base, and wherein the means for applying a discomfort-evoking stimulation is/are adapted to apply a stimulus which is gradually increased, the storage means being adapted to store a stimulation level at a moment in time corresponding to the first and the second user operation, respectively.

The calibration threshold value may be a predetermined or pre-established value, which is stored permanently, e.g. the value zero.

In a sixth aspect, the invention relates to use of measuring nociception for determining the level of WSS and/or DRS in a subject.

The present assignee's previous international patent applications WO 2005/084529 and WO 2006/092146 disclose systems, methods and uses which are all applicable when carrying out the present invention. Hence, the present invention includes aspects which utilise the determination methods and systems disclosed in WO 2005/084529 and WO 2006/092146 as well as improvements to the methods, uses and systems disclosed in WO 2005/084529 and WO 2006/092146. Notably, all teachings in WO 2005/084529 and WO 2006/092146 apply mutatis mutandis to determination of WSS levels according to the present invention and it will be understood that the methods for determination of sympathetic tone and/or level of stress taught in WO 2005/084529 and WO 2006/092146 may equally well be applied when determining the level of WSS according to the present invention.

However, the present invention also demonstrates that WSS and/or DRS in an animal (as well as the the level of stress) can be determined by measuring the noxious withdrawal reflex (NWR) in an animal (including a human being)—notably, previous findings using the methods and systems of WO 2005/084529 and WO 2006/092146 which established methods of determining stress levels and levels of sympathetic tone has according to the present invention also been demonstrated to correlate with the threshold value of NWR.

### BRIEF DESCRIPTION OF THE DRAWINGS

The invention is explained in detail below with reference to the drawings, in which

FIG. 1 shows the position of the sympathetic tone-neutral point anteriorly on the upper side of the clavicle, and the position of the sympathetic tone-dependent point, Per 1, the grey-shaded area, between the nipple and the anterior axillary fold, the black dot in the grey-shaded area indicating the most frequently used point within Per 1 according to the invention.

FIG. 2 shows the position of the sympathetic tone-neutral point posteriorly on the spinal column, more precisely at TH 10-11 and the position of the sympathetic tone-dependent point posteriorly corresponding to TH 3-6 in the area between the shoulder blades.

FIG. 3 shows the position of the sympathetic tone-neutral point anteriorly on the upper side of the clavicle, and the position of the sympathetic tone-dependent point, C.V. 17, the grey-shaded area, in the middle of the sternum, the black dot in the grey-shaded area indicating the most frequently used point within C.V. 17 according to the present invention.

FIG. 4 shows the position of the sympathetic tone-neutral point, anteriorly, on the upper side of the clavicle, and the position of the sympathetic tone-dependent point, St 18, the grey-shaded area, between two ribs below the nipple, the black dot in the grey-shaded area indicating the most frequently used point within St 18 according to the invention.

FIG. 5 shows a system according to the invention, the parts of the system being shown as integrated in one and the same apparatus. The apparatus includes a pressure base with a contact face adapted to exert an outer compressive force on a human's body, a sensor for measuring the compressive force exerted on the body by the pressure base. The apparatus includes a read-out unit for displaying the read-out value.

FIG. 6 shows an end portion of an apparatus of a system according to the invention, the end portion comprising a contact face adapted to exert a force on a human's body and a sensor for measuring the force exerted on the body.

FIG. 7 shows a principle diagram of an embodiment of the system of FIG. 5.

FIG. 8 shows a stress factor diagram.

FIG. 9 shows a flowchart with the basis principle for data treatment.

FIG. 10 shows a cross-section of the mechanical parts of the actuator for an embodiment of the system of the present invention for mechanical stimulation using vibration.

FIG. 11 shows the magnetic field created by the actuator for an embodiment of the system of the present invention for mechanical stimulation using vibration.

FIG. 12 shows different parts of the actuator in FIG. 10.

FIG. 13 shows a principle diagram for the system of FIG. 10.

FIG. 14 shows a display of the actuator control (adjustment of frequency and stroke).

FIG. 15 shows temperature set-point as function of time.

FIG. 16 shows stress factor as a function of time.

FIG. 17 shows a simplified diagram for the hardware design of an embodiment of the system of the present invention for thermal stimulation.

FIG. 18 shows time pulse and power pulse At as a function of time.

FIG. 19 shows the results of a study with 27 opera singers with measurements according to the invention (SA value) as a measure of changes in performance related stress. In the study measurements of MBP, PRP, Saliva cortisol and SA were made prior to, during and after song performance. Changes in SA values correlated significantly to changes in PRP (r=0.5; p<0.005), MBP (r=0.4; p<0.01) and Cortisol (r=0.3; p<0.05).

FIG. 20 shows the results of a study with 14 opera trainees with measurements according to the invention (SA value) as a measure of changes in stress in relation to mental stress. Measurements of PRP and SA were performed at the following intervals: After 10 minutes of rest, followed by challenge (mental stress test). The figure depicts changes in PRP and SA (delta SA vs. delta PRP). SA and PRP change is positive correlated during mental stress.

FIG. 21 shows the results of a study with 14 opera trainees with measurements according to the invention (SA value) as a measure of changes in stress as a result of a cycling test. Measurements of PRP and SA were performed at the following intervals: After 10 minutes of rest, followed by challenge (bicycling test). The figure depicts changes in PRP and SA (delta SA vs. delta PRP). SA and PRP change is negative correlated during physical performance.

FIG. 22 shows the results of a study with 14 opera trainees with measurements according to the invention (SA value) as a measure of changes in stress as a result of singing. Measurements of PRP and SA were performed at the following intervals: After 10 minutes of rest, followed by challenge (song performance). The figure depicts changes in PRP and SA (delta SA vs. delta PRP).

FIG. 23 shows the results of at study with 112 consecutive patients with verified ischemic heart disease. The method used was cross sectional registration of use of beta-blockage medication and measurement of SA-value. The results showed no significant difference in SA measurement between patients with and without beta-blockage (p>0.1).

FIG. 24. Opera solo singers (N=26), changes during a premiere performance in mean values of PT(FIG. 24A), HR (FIG. 24B), PRP (FIG. 24C), MBP (FIG. 24D) and salivary cortisol (FIG. 24E) with 95% confidence limits. PT: 34, 45, 32 (p<0.0001); HR: 69, 84, 72 (p<0.001); PRP: 9828, 12053, 9865 (p<0.005); MBP: 103, 107, 101 (p<0.001); salivary cotisol: 4, 5, 3 (p<0.05)

FIG. 25. Changes in PRP and PT.

(A) Opera solo singers (N=26), changes in PRP and PT during a premiere performance (r=0.54, p<0.0001).

(B): Opera trainees (N=27), changes in PRP and PT during a bicycle exercise test, (r=−0.70, p<0.0001).

FIG. 26. Occurrence of non-cognitive noxious withdrawal reflex (NWR) in patients: PT<30 (N=46); 30<=PT<60 (N=64); PT>=60 (N=43).

FIG. 27. Reliability test.

(A) First time measurement of PT conducted by HCP in 82 healthy people (r=0.94, p<0.0001.

(B) PT measurement conducted by HCP in 181 consecutive patients in a medical outpatient clinic (r=0.97, p<0.0001. (C): Self PT measurements by 36 NON-HCP (r=0.95, p<0.0001).

FIG. 28. An example of well conducted peak performance and full degree of elasticity.

SO (=WSS, shown with ⋄) is high and high at the singing audition, which is good as this is a real-life important situation with transient stress, when compared to mental and physical exercise test, which have no real-life importance. Note: the SO level may be elavated at the measurement before the singing audition if the measurement is conducted in the very last minutes before the audition.

A good level of elasticity is indicated as an increase in both SO and PRP (shown with □) at singing audition as well as in mental stress test. In addition, the increased levels of both SO and PRP are both quickly normalised with the stress situations are over.

At the bicycle test, SO decreases significantly as PRP increase, and vice versa when bicycling is over—again indication full elasticity.

FIG. 29. An example with possible insufficient peak performance and decreased level of elasticity

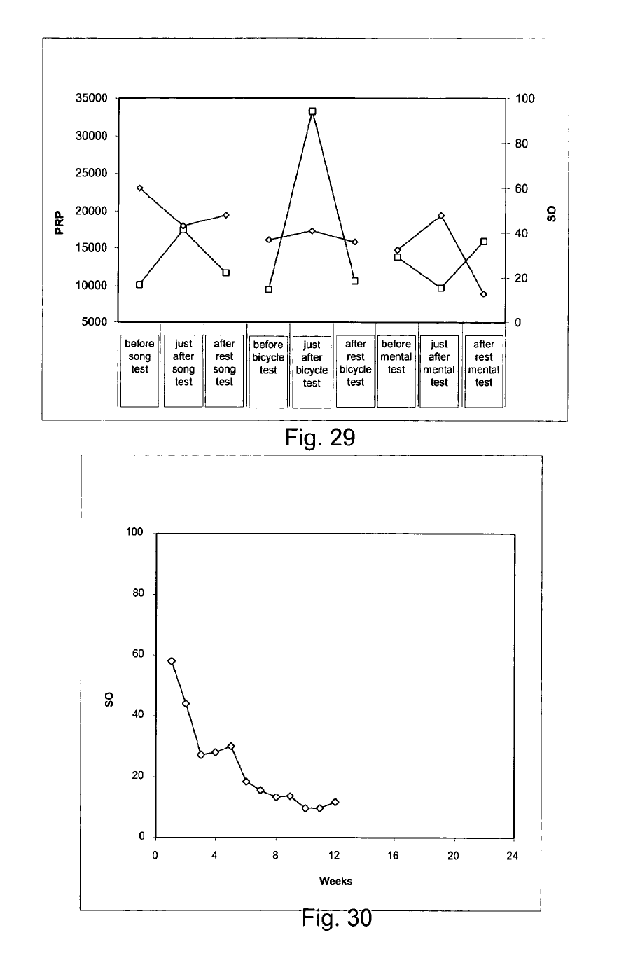

The curves uses the same symbols for SO and PRP as does FIG. 28.

Peak performance may be insufficient during mental stress test. Although the stress level increases during the test, the work of the heart decreases, which will lead to an insufficient task performance. When the test is over, the stress level goes down, while the work of the heart increases, indicating lack of elasticity and a paradox cardiophysiological response to stress.

After the singing audition, SO increases while PRP decreases as during the mental test. This supports the lack of elasticity, as the physical body is resting after the singing audition, the stress level increases—indication lack of mental restitution.

At the bicycle test, minor changes in SO are found, despite major changes in PRP—thus an additional indicator for insufficient elasticity.

The work of the heart is at a elevated 30% compared to a fellow during, and during rest as well

FIG. 30. Development in level of stress measured as WSS at wake-ups during 12 weeks of self-measurement.

SO is initially at an elevated level (normal values for men <15). Furthermore as the SO measurement are consistently high during the first weeks, it may be concluded, that the person is exposed to persistent stress.

By daily and systematic use of the Selfcare© program as taught herein, the general level of SO (=WSS) ultimately arrived at a normal level.

### BEST MODE(S) FOR CARRYING OUT THE INVENTION

Definitions

Prior to a detailed description of the invention, specific phrases relating to the aspects of the inventions are defined:

The phrase “stress” denotes a strain condition in which the strain exceeds the resources of the body. Stress can be pleasant, but can also be unpleasant and sometimes dangerous to life. It is essential to distinguish between two forms of stress: transient and persistent. “Transient stress” (=“acute stress”) denotes the physiological state of preparedness, a state which is automatically induced in the body through neural/hormonal signals from the brain when a threat is perceived. The level of stress depends on the balance between the individual expectancies of the outcome of the stimulus and the resources available. When the challenge/threat is over, homeostasis is re-established. If the person has the necessary resources available, the situation may be perceived in a positive manner. If the situation represents a state in which the strain exceeds the resources of the body, the situation will most likely be perceived negatively. If this latter situation situation continues, the resssources of the organism may become taxed and the performance of the body impaired. An increased level of stress is expressed as an increased sympathetic tone. However, an increase in sympathetic tone may not express an increased level of stress as it is seen in mild physical exercise.

The phrases “persistent stress” or “chronic stress” are used synonomically, and are a dysfunction of the neural/hormonal processes of the brain due to a prolonged exposure to the neural processes/hormones involved in transient stress, and with insufficient restitution in between. It can be harmful to our health.

The phrase “sympathetic tone” denotes the level of activity in the sympathetic part of the nervous system and is usefull in the measure of a person's potential to perform optimally both physically and mentally.

The phrase “acute stress” denotes a condition in which a person over a short period of time, typically hours/days, has experienced situations which have caused an increased activity in the sympathetic nervous system due to stress.

The phrase “transient stress” is used synonomic to “acute stress”.

The phrase “persistent stress” is used synonomic to “chronic stress”. The term persistent stress is preferred, as it neutral in respect to disease terminology, in contrast to “chronic” which is used for a prolonged disease related condition. This is found of importance as mild persistent stress may not unhealthy or harmfull to your health, if subsequent restitution takes place. To allow consistency in terminology, the notion “transient stress” is preferred to “acute stress”.

The phrase “accumulated stress” denotes a condition in which a person over a long period of time, typical weeks/months/years, has experienced situations which have caused an increased activity in the sympathetic nervous system.

The phrase “clinical stress” denotes a condition in which stress triggers clinical symptoms.

The phrase “physiological stress” denotes the determination of sympathetic tone without distinction between change in sympathetic tone due to psychological stress (=stress) or due to physical stress (=exercise induced stress)

The phrase “psychological stress” denotes stress, and is mainly used, when a distinction to physical stress is important.

The phrase “physical stress” denotes an increase in sympathetic tone due to physical exercise, only. However, it should be emphazised that this denotion holds for mild to moderate psysical exercise, only as excessive physical exercise may lead to psychological stress.

The phrase “mental stress” denotes an increase in sympathetic tone due to mental exercise, only. However, it should be emphazised that the conduction of a mental stress exercise may lead to stress in some persons and under somee circumstances, and may not lead to stress in other persons or in the same persons under other circumstances. Whether a mental stress test leads to stress or not depends on the personal and circumstantial balance between the individual expectancies of the outcome of the stimulus and the resources available.

The phrase “stimulation” denotes any type of stimulation which activates the skin's mechanoreceptors, thermoreceptor and/or nociceptive receptors. Stimulation may be provided as mechanical, thermal, electrical, radiation and/or chemical stimuli. A mechanical stimulation may for instance be provided by means of a compressive force and/or by means of vibration. A thermal stimulation may for instance be provided by means of cold and/or heat. An electrical stimulation may for instance be provided by provided by means of alternating current and/or direct current. Radiation stimulation may for instance be provided by means of an applied infrared, visible and/or ultraviolet light or combined spectra thereof, e.g. a laser, light-emitting diode, infrared, ultraviolet and/or white light source. Chemical stimulation may be provided by means of an organic and/or an inorganic compound.

The phrase “sympathetic tone-neutral point” denotes a point on or in the body in which the sensitivity to an applied stimulation is independent of the activity level of the sympathetic nervous system and/or to the level of acute stress. Also covered by the expression is a point on or in the body where increased sympathetic tone and/or level of stress causes a higher threshold for sensitivity or nociception in said point.

The phrase “sympathetic tone-dependent point” denotes a point on or in the body in which the sensitivity to an applied stimulation is dependent on the activity level of the sympathetic nervous system, in the sense that increased sympathetic tone or an increased level of stress causes the point to exhibit a lowered threshold for sensitivity and/or nociception.

As will be apparent from the present disclosure, the anatomic location of the sympathetic tone-neutral and sympathetic tone-dependent points are not essential. In cases where stimulation is performed utilising an implanted device, the point in question will be “in” the body, whereas use of a hand-held device typically will aim at stimulating a point “on” the body.

The phrase “threshold value of the simulation” denotes at which intensity the applied stimulation is to be applied to a given point in order for the person to perceive the applied stimulation as not pleasant, more specifically as unpleasant or as pain.

The phrase “threshold value of pressure sensitivity” denotes at which intensity the applied pressure is to be applied to a given point in order for a person to perceive the applied pressure as not pleasant, more specifically as unpleasant or as pain.

The phrase “nociception threshold value” denotes the threshold at which the person in the respective point perceives a stimulation as nociceptive, i.e. as tissue-damaging. The expression also includes stimulation which is perceived as uncomfortable by the person.

The phrase “substantially at the same time” denotes that the measurements, e.g. of the calibration threshold value and the stimulation threshold value, are performed within a period of a few minutes, e.g. one minute, two minutes, three minutes, five minutes, ten minutes, fifteen minutes.

The phrase “significantly lower” means that the nociception threshold value in a sympathetic tone-dependent point is no more than 85%, particularly no more than 80%, and most particularly no more than 75%, of the threshold value in a sympathetic tone-neutral point.

The phrase “system for applying and measuring a stimulation” denotes a system, e.g. an apparatus or several apparatuses, which are able to apply and measure a stimulation.

The phrase “pressure-sensitive apparatus” denotes an apparatus which is able to apply and measure a pressure.

The phrase “marker” denotes a means marking a measuring point.

The phrase “measuring point” denotes a point whose threshold value of the stimulation at an applied stimulation is either neutral or dependent on the sympathetic tone.

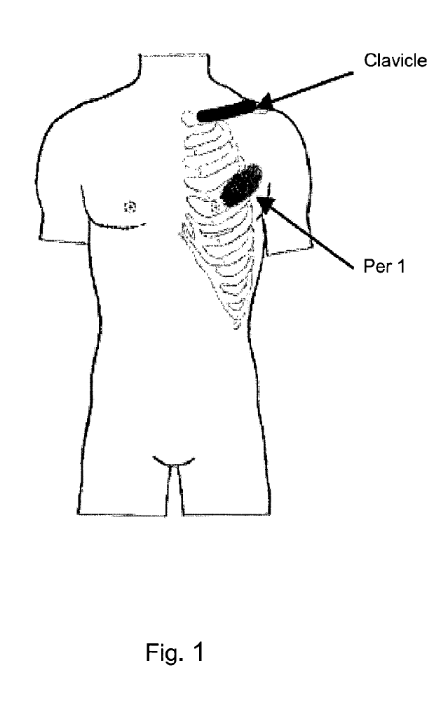

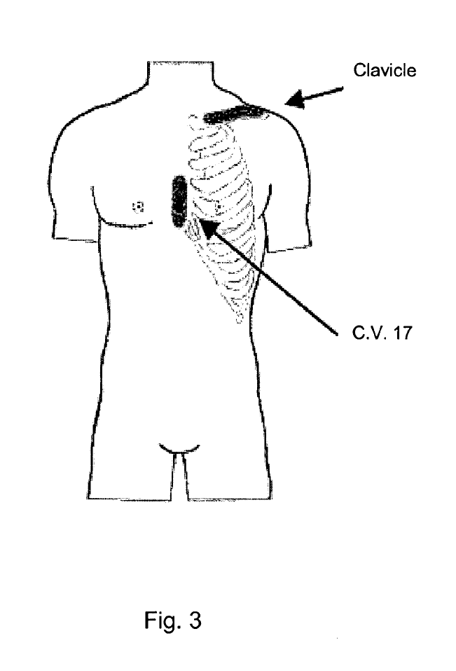

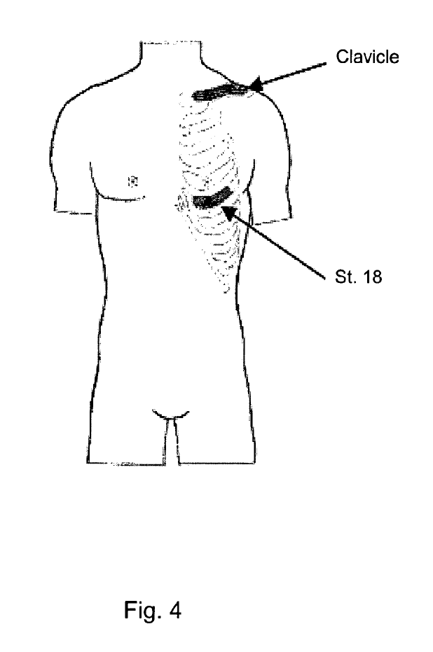

The expressions “C.V. 17”, “Per 1” and “St 18” denote meridian points pursuant to conventional Chinese theory (Beijing College of Traditional Chinese Medicine: Essentials of Chinese Acupuncture, Beijing Foreign Languages Press, 1980). C.V. is a conception vessel; Per is the pericardium and St is the stomach. The C.V. 17 point, the grey-shaded area, is shown in FIG. 3, where the most frequently used point according to the present invention within C.V. 17 in the grey-shaded area is indicated by the black dot. The Per 1 point, the grey-shaded area, is shown in FIG. 1, where the most frequently used point according to the present invention within Per 1 in the grey-shaded area is indicated by means of the black dot. The St 18 point, the grey-shaded area, is shown in FIG. 4, where the most frequently used point within St 18, in the grey-shaded area, is indicated by means of the black dot. The described points, C.V. 17, Per 1 and St 18, are well-defined according to their Chinese names and are in form of points on the surface of the body. In FIGS. 1, 3 and 4 grey-shaded areas are provided to mark that an actual area is to be examined and that the point merely is defined by its quality as being the most sore point when stimulated. This also means that the point may be outside the grey-shaded area marked on the drawings. In reality, the point may be at any position within the portion of the skin corresponding to the nerve supply to the heart of the sympathetic nervous system (as for instance stated in the following references: Rutherford J. D., Braunwald E. & Cohn P. F., “Chronic heart disease”; Braunwald E., ed. “Heart Disease. A textbook of Cardiovascular Medicine”. Philadelphia: W.B. Saunders Company, 1988; 1314-67; Williams P. L., Warwich R., Dyson M. & Bannister L. H., eds. Gray's Anatomy. New York: Churchill Livingstone, 1989; 723-1168; Mann, F., “Textbook of acupuncture”, William Heinemann medical books, London 1987; 57-64).

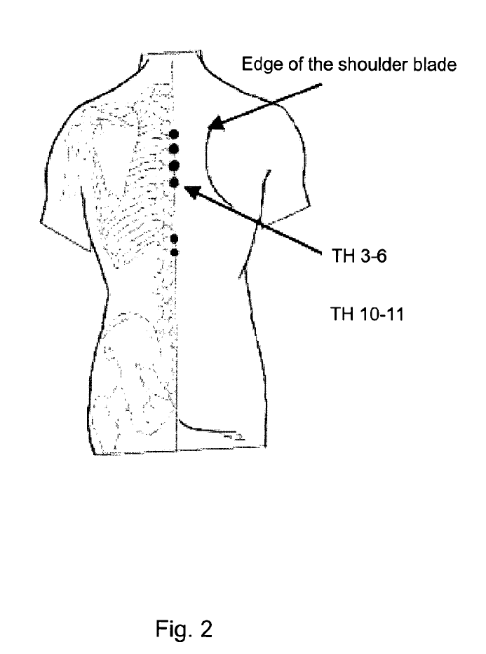

The expressions “TH 3-6” and “TH 10-11” denote the spinous processes 3-6 and the spinous processes 10-11, respectively, on the thoracic vertebrae of the same numbers. The spinous processes are the parts of the spinal column which feel like hard projections. The spinous processes 3-6 and the spinous processes 10-11 are shown in FIG. 2, the spinous processes 3-6 being the uppermost four black dots on the spinal column and the spinous processes 10-11 being the two lowermost dots on the spinal column.

“Warning system sensitivity” or “WSS” denotes a CNS controlled alerting system which acts through the sympathetic nervous system to establish an enhanced performance or alertness in situations of stress. Thus, an increase in WSS is always accompanied by an increased sympathetic tone, whereas increased sympathetic tone not necessarily is accompanied by an increase in WSS (e.g. when the increase in sympathetic tone is due to a physiological need to increase sympathetic tone such as in mild to moderate physical exercise). The findings of the present inventions indicate that a general increase in WSS is accompanied with an increased tenderness (lowering of nociceptive threshold) in sympathetic tone dependent points on or in the body, meaning that a measurement of the present invention which demonstrates such an increase in tenderness provides an indication of increased sensitivity of WSS.

The phrase “Defense reaction/reflex sensitivity” or “DRS” denotes a CNS controlled non-cognitive reflex mediated defense system, linked to the sympathetic nervous system and to WSS in order to establish enhanced defense reactions. Thus, an increase in DRS is always accompanied by an increased sympathetic tone and WSS, whereas increased sympathetic tone not necessarily is accompanied by an increase in DRS (e.g. when the increase in sympathetic tone is due to a physiological need to increase sympathetic tone such as in mild to moderate physical exercise). The findings of the present inventions indicate that a general increase in DRS is accompanied with an increased tenderness (lowering of nociceptive threshold) in sympathetic tone dependent points on or in the body, meaning that a measurement of the present invention which demonstrates such an increase in tenderness provides an indication of increased sensitivity of DRS.

The phrase “noxious withdrawal reflex” during measurement is used for the presence of in-voluntary muscle contractions in the region of the eye, cheeks (=startle reflex) or in the flexor muscles of the neck and upper extremity.

The phrase “Elasticity of the nervous system” denotes the ability of the organism to exercise the appropriate adjustments of function in response to changes in circumstances, ie. the size of the response to a stressfull situation and the rate of recovery. A god elasticity denotes that the organism adjusts quickly and adequatly to such changes. In respect to a specific situation of transient stress, this means that the stress response is activated sufficiently at the peak of the performance and that homeostasis is reestablished quickly, when the situation is over. The degree of elasticity is a usefull measure for level of persistent stress, with an increasingly lack of elasticity representing an increase in level of persistent stress.

### Embodiments of the Invention

For ensuring the optimum utilization of a person's total resources, in a given stress-evoking situation it is vital that this can be ensured by measuring the functional level of the nervous system at any given time.

The method according to the invention provides humans with such a tool in form of a method of determining the immediate (here-and-now) activity level of the sympathetic nervous system:
1) A low measured value denotes a low activity level in the sympathetic nervous system and is thus the best possible base for coping optimally with a future stressful situation.
2) A single high value provides the user with the information that the person—physiologically speaking—has mobilised the resources of the organism in a so-called “stress phase” with a view to coping with a situation which the brain perceives as dangerous or potentially dangerous.
3) Repetitive high measurements provide the user with the information that the person is in a prolonged “stress phase”, which in the long run may tax the resources of the organism and provide a basis for a reduced functional level mentally, physically, emotionally and socially.
4) Varying high and low measurements provide the user with the information that the person is in situations in which the “stress phase” alternately is activated and inactivated. As a result a possibility for learning and awareness exists.

In a specific embodiment, the method is thus linked to special tools and educational programmes which based on the actual measurement can teach the person how to prevent and treat negative stress and teach the person what specifically increases/reduces the stress level in him/her.

The present invention is the result of intensive research in methods for determining the activity level of the sympathetic nervous system and thus a person's potential to perform optimally both physically and mentally and neatly solves the problems of the known methods of determining the sympathetic tone, which is precisely a measure of the activity level of the sympathetic nervous system, here referred to as psychological stress, in contrast to physical stress.

According to the present invention, it has surprisingly been found that points on the body exist, whose threshold value of a stimulation when this is applied to the point is sympathetic tone-neutral or even capable of reacting by analgesia, while other points are sympathetic tone-dependent, cf. the definition above—it has further been found that this is only the case when the sympathetic tone is linked to the status of WSS and/or DRS, whereas an increase in sympathetic tone which is the consequence of e.g. simple mild to moderate physical exercise does not lead to an increase in tenderness in the sympathetic tone-dependent areas/points. In other words, certain points exist, where the sensitivity to an applied stimulation is independent of the activity level of the sympathetic nervous system or where an increased sympathetic tone/level of stress induces analgesia, while there are other points, where the sensitivity to an applied stimulation is dependent on the activity level of the sympathetic nervous system (when dependent on WSS/DRS) in the sense that these points can become hyperalgesic in response to an increased sympathetic tone and/or level of stress and/or level of WSS/DRS. Hence, according to the invention, a person's sensitivity to the applied stimulation in a sympathetic tone-dependent point increases when the activity level of the sympathetic nervous system also increases in connection with an increase in WSS/DRS. This realization is surprising in that it has previously been described that stress and thus increased sympathetic tone generally increases the tolerance to pain (Amit and Galina, Physiol. Rev. 66: 1091-1120, 1986). The identified sympathetic tone-dependent points thus respond differently than what is known for the body in general. In fact, the identified sympathetic tone dependent points are those where stress and/or increased sympathetic tone can be demonstrated to effect a hyperalgesia (i.e. increased sensitivity or increased nociception), something which is in contrast to the previously demonstrated analgesia induced by stress or increased sympathetic tone.

By measuring which intensity of an applied stimulation is necessary to obtain a threshold value of the stimulation in a sympathetic tone-neutral point and comparing this with the necessary intensity of an applied stimulation to obtain the same threshold value of the stimulation in a sympathetic tone-dependent point, a physiological measure of WSS/DRS (and sometimes of the sympathetic tone) and thus of the person's psychological stress or immediate stress level is obtained. The measurement may be considered as a here-and-now determination of the level of the the WSS.

When using the method according to the invention to determine the chronic, immediate or acute stress level and/or WSS/DRS status, the method provides the person with a tool for adjusting the stress state and thereby optimizing his/her performance. An acute increase in the activity level of the sympathetic nervous system may have a beneficial effect on the person's performance. It is thus well known that an increased adrenaline level may be beneficial for optimising the performance. The method according to the invention is thus used to adjust the sympathetic tone such that an increased activity level of the sympathetic nervous system is obtained resulting in a beneficial effect.

Also important is the ability of the method of the invention to render it possible for a person to identify factors in his/her environment or lifestyle which result in non-recognized increases or decreases in WSS/DRS (e.g. observed as transient or persistent stress) because the method of the invention allows for repeated determinations of the level of WSS/stress—if a person runs a diary during a period with such repeated determinations, it becomes possible to correlate events from daily life with the determination, thus facilitating identification of stress-inducing factors as well as stress-reducing activities/actions.

A method of determining the immediate activity level of the sympathetic nervous system and/or of the level of stress and/or level of WSS and/or DRS is thus provided, said activity level being significant to the person's ability or potential to perform optimally. The method may be of a diagnostic nature and have diagnostic value, but it may also be described as a prognostic method in that it provides the person with a prognosis of the person's immediate potential to perform optimally and thus prevent suboptimal performance. As mentioned in the introduction, the sympathetic nervous system mobilises the resources of the organism in a so-called “stress response” such that an actual dangerous situation/challenge is handled in the best possible manner. This means that mentally the person thinks faster and more clearly at the same time as the ability to focus his/her thoughts is increased. For supporting this purpose, irrelevant sense impressions are effectively impeded. Physically the body responds by lowering the response time, increasing the muscle strength, sharpening the senses, and optimizing the coordination between thought and motor skills.

A test among 146 healthy randomly selected persons revealed that half (41) of the 79 persons which were heavily stressed, i.e. in a stress group level 3 as defined below, perceived themselves as having no/little stress As a result, the present method and measuring tool are of great practical value by providing the person with vital information to which the person otherwise would not have access.

As mentioned above, the method according to the invention may also be used to record the activity level of the sympathetic nervous system and thus the WSS and/or DRS on a long-term basis and may thus be effective for preventing in time that such a state lead to stress-related complications. Such recordings may for instance be forwarded to a central register/data centre monitoring the data of the individual person and in time sends a warning back to the person, whereby the said complications can be avoided.

According to the invention the stimulation sensitivity in the points may vary highly from one person to the next and a single determination of the sympathetic tone/WSS/DRS for a given person cannot necessarily be related to the actual stress state of the person unless the person's normal state is known. It may thus be necessary to supplement the single determination of the sympathetic tone/WSS/DRS with additional information. A low activity level of the sympathetic nervous system/WSS/DRS and a small difference between the repeated determinations thus indicates an optimum prognostic utilization of the person's resources. A high activity level and smail difference difference between repeated determinations is, however, not unambiguous, but requires additional information. A situation, where a high activity level is determined by means of the method, may thus occur, while an additional analysis by means of a stress form (questionnaire) reveals that the person displays no sign of clinical stress. In this case the measurement may reflect: 1) transient stress and not persistent chronic stress or it may reflect 2) persistent stress with the person not being conscious of the clinical stress symptoms. The latter may be observed in persons with severe levels of persistens stress. Supplementary tests with respect to elasticity may distinguish between the two situations: high level of elasticity indicates that the persons is exposed to transient stress, while insufficient elasticity may indicate that the person is exposed to persistent stress (see FIGS. 28-30). A situation, where high values of WSS/DRS are combined with low values of WSS/DRS, while a short time period, indicate that the high measurement reflect situations associated with transient stress, and that the level of persistent stress is low (see FIG. 28-29).

Nevertheless, for many practical purposes it is convenient to determine calibration threshold values representing various levels in a population (for example with respect to age, sex and ethnic background), since this provides for the possibility of ascertaining the stress level and/or sympathetic tone in an individual by performing one single measurement of the stimulation threshold value, thus rendering the need for the measurement of a calibration threshold value unnecessary.

Thus, the calibration threshold value may, instead of being a value obtained from a measurement, be a predetermined or preestablished value, i.e. a value based on previous observations from one person or a large group of persons.

According to the invention a stimulation may be any type of stimulation activating the skin's mechanoreceptors, thermoreceptor and/or nociceptive receptors, or equivalent receptors inside the body (e.g. in the periostal tissue). Stimuli may be provided as mechanical, thermal, electrical, radiation and/or chemical stimulus. A mechanical stimulation may for instance be provided by means of a compressive force and/or by vibration. A thermal stimulation may for instance be provided by means of cold and/or heat. An electrical stimulation may for instance be provided by means of alternating current and/or direct current. Radiation stimulation may for instance be provided by means of an applied infrared, visible and/or ultraviolet light or combined spectra thereof, e.g. a laser, light-emitting diode, infrared, ultraviolet and/or white light source. Chemical stimulation may be provided by means of an organic and/or an inorganic compound.

It is also possible to interact directly with polymodal sensor cells or efferent motor nerve cells, cf. the detailed discussion below. These cells may be stimulated with AC or DC, neurotransmitters or chemicals that are capable of triggering firing of a nerve cell.

The recorded physiological measure of the sympathetic tone is a total measure of the sum of the person's acute stress level and the person's accumulated stress level—i.e. the WSS and/or DRS. The method according to the invention also allows for the recordal of the effect of any intervention/stress-reducing initiatives.

The threshold value of the stimulation is obtained when the person, to whom astimulation is applied to a specific point no longer perceives the applied stimulation as comfortable, more specifically when the person perceives the applied stimulation as unpleasant or as pain. Again, a correlating measurement may be obtained by determining the firing threshold for polymodal sensor cells or efferent motor nerve cells in a sympathetic tone dependent point.

Sympathetic tone-neutral points may also be denoted as calibration points. These points may be located anteriorly on the upper side of the clavicle and posteriorly on the spinal column, specifically denoted as TH 10-11. The points may also be located anywhere on a finger or a toe, although preferably on the dorsal side of a finger or a toe.

Sympathetic tone-dependent points may also be denoted as recording points. These points may be located anywhere on/in the skin which innervationally correspond to the nerve supply of the sympathetic nervous system to the heart, e.g. anteriorly, to which three points are connected: C.V. 17 in the middle of the sternum, ST 18 between two ribs below the nipple and Per 1 between the nipple and the anterior axillary fold and posteriorly corresponding to TH 3-6 in the area between the shoulder blades. According to an embodiment the most sore point of the said points is preferably chosen, such a point rendering the most accurate representation of the activity level.

The present invention allows for an overall measure of a person's acute stress over a short period of time, e.g. hours/days, as well as of the accumulated stress over a long period of time, e.g. months/years.

The distinction between transient and persistent stress may be carried out by means techniques known to the person skilled in the art. These techniques include without being limited thereto: conversations about the person's physical and mental state or other manners in which the state can be elucidated optionally by filling in a stress form (questionnaire). Furthermore, the causes of stress can be found by means of techniques which are known to the person skilled in the art. These techniques include without being limited thereto: a conversation about the person's physical and mental state or other manners in which the state can be elucidated optionally by filling out a stress/resource balance sheet. These techniques include without being limited thereto: repeated measurent of the WSS/DRS by the individual in combination with diary with respect to This is briefly discussed above as supplement of additional information to the determination according to the invention. It is also possible to monitor a person's activity level over a period of time (by continously measuring heart rate, blood pressure etc.) as well as letting the person record a detailed diary with respect to 1) WSS/DRS, 2) events, 3) thoughts, 4) emotions and 5) personally identified stress-related clinical symptoms, which in combination with cognitive processing of the achieved information will provide an answer to distinguish between transient and persistent stress.

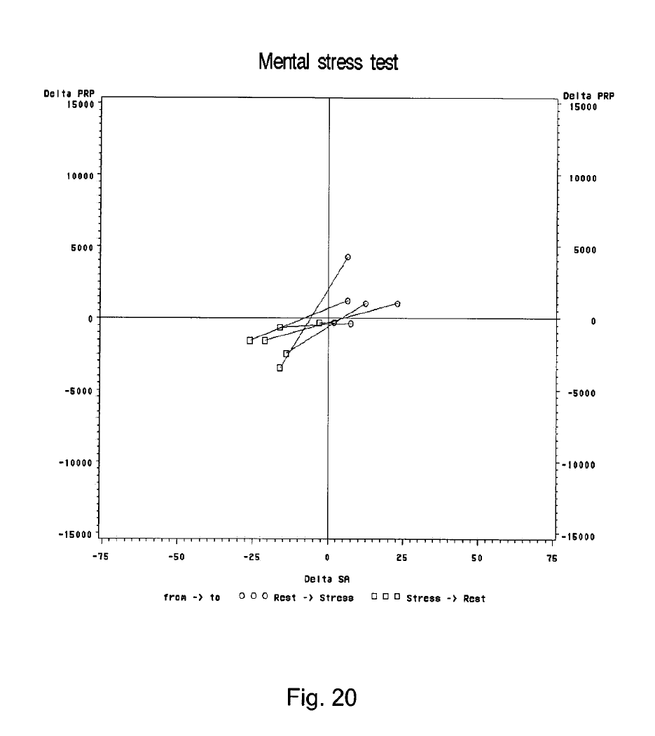

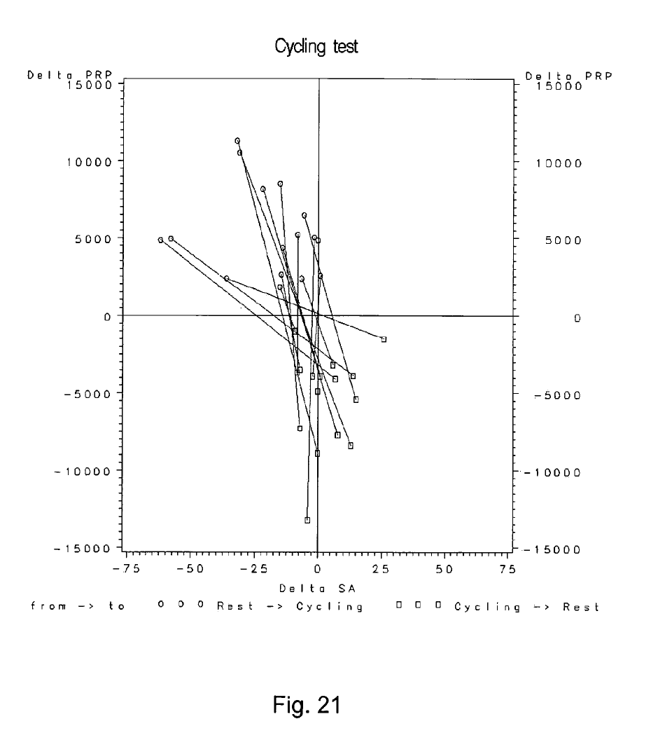

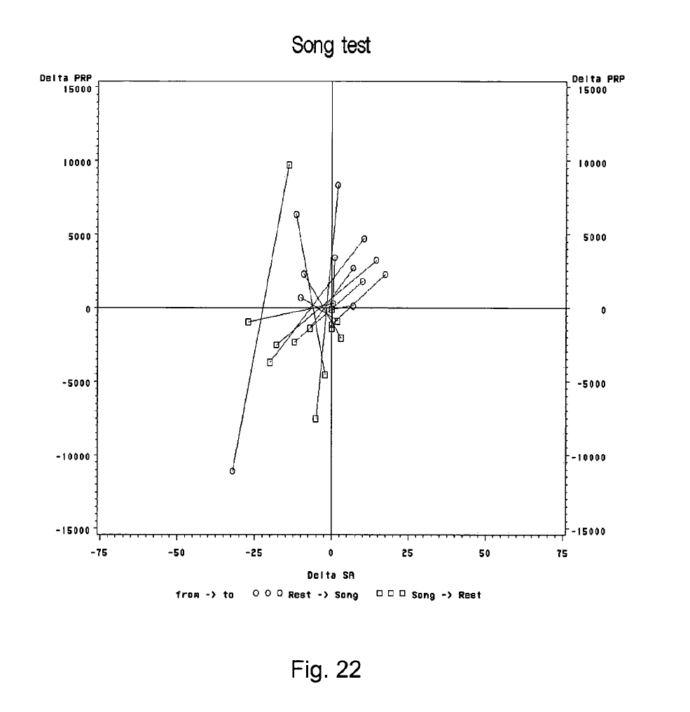

The inclusion of specific test for elasticity may also be included. These techniques include without being limited thereto: Conduction of mental stress test, physical exercise test (for example a bicycle exercise test) and/or performance tests relating to the professional life of the person (see FIG. 20-30). The aspects of the tests reflects the elasticity: the size of the stress response with respect to the stimulus and the rate of the subsequent recovery.

The first clinical signs of chronic stress are fatigue and increased muscle tension in the muscles of the motor apparatus. It can manifest itself as for instance headache and back, shoulder and neck pains. This state is harmless and is experienced in many of the situations which are perceived as positive stress.

In case of prolonged stress loads, additional symptoms are triggered in the portions of the nervous system which are not under the power of the will, viz. the autonomic nervous system. These symptoms may for instance manifest themselves as moodiness, stomach ache, palpitations and lack of concentration.

If the stress load is further exacerbated, additional symptoms to the above symptoms are triggered in the portions of the nervous system which are under the power of the will. A person is for instance no longer able to control his/her anger or irritability and the social behaviour is negatively affected.

The measuring of which intensity of an applied stimulation is necessary to obtain a threshold value of the stimulation can be determined by using a system capable of measuring the intensity of the applied stimulation. One example of such a system for measuring an applied stimulation is a system capable of measuring an applied mechanical stimulus, an applied thermal stimulus, an applied electrical stimulus, an applied radiation stimulus and/or a chemical stimulus. A system for measuring an applied mechanical stimulus may for instance be an apparatus for measuring an applied compressive force, said apparatus for instance being a manometer. After tests with for instance an apparatus capable of measuring an applied compressive force, the measuring of which intensity of an applied compressive force is necessary to obtain a threshold value of the pressure sensitivity can be performed with a finger.

The method according to the invention may furthermore be used as a measure of the effect of various initiatives. These initiatives, which may or may not be related to professional health treatment, may for instance include initiatives corresponding to the situations perceived as stress-evoking.

The method according to the invention may be carried out by a person other than the person being measured or by the person being measured. The most accurate measurement is obtained when the person himself/herself performs the determination.

Comprehensive studies have now revealed that the levels of activity of the sympathetic nervous system (Level 0-3) can be correlated in the following manner to which stimulation in form of an applied compressive force is necessary to obtain a threshold value of the pressure sensitivity in a sympathetic tone-neutral in relation to which stimulation in form of an applied compressive force is necessary to obtain the same threshold value of the pressure sensitivity in a sympathetic tone-dependent point:

Level 0: When the applied compressive force at a threshold value of the pressure sensitivity in a sympathetic tone-dependent point exceeds or is equal to 80% of the applied compressive force at the same threshold value of the pressure sensitivity in a sympathetic tone-neutral point.

Level 1: When the applied compressive force at a threshold value of the pressure sensitivity in a sympathetic tone-dependent point is between 55% and less than 80% of the applied compressive force at the same threshold value of the pressure sensitivity in a sympathetic tone-neutral point.

Level 2: When the applied compressive force at a threshold value of the pressure sensitivity in a sympathetic tone-dependent point is between 30% and less than 55% of the applied compressive force at the same threshold value of the pressure sensitivity in a sympathetic tone-neutral point.

Level 3: When the applied compressive force at a threshold value of the pressure sensitivity in a sympathetic tone-dependent point is less than 30% or the applied compressive force at the same threshold value of the pressure sensitivity in a sympathetic tone-neutral point.

The above ratios between the level of activity of the sympathetic nervous system and the applied compressive force at a threshold value of the pressure sensitivity in a sympathetic tone-dependent point in relation to the applied compressive force at the same threshold value of the pressure sensitivity in a sympathetic tone-neutral point may vary from one person to the next. In a few cases the variation may be up to about 90%.

In the same person, the measurements may furthermore vary between the different sympathetic tone-dependent points and between the different sympathetic tone-neutral points. In order to obtain the most accurate determination of the activity level of the sympathetic nervous system it is thus vital to choose a sympathetic tone-neutral point, which is not sensitive due to other factors.

The above correlation between which applied compressive force is necessary to obtain a threshold value of the pressure sensitivity in a sympathetic tone-neutral point in relation to which applied compressive force is necessary to obtain the same threshold value of the pressure sensitivity in a sympathetic tone-dependent point has also been found to apply when a thermal, electrical, radiation or chemical stimulus is used. As an example it has been found that when an applied compressive force is used, a sympathetic tone-dependent point is more sensitive to for instance heat and cold, the heat for instance being transferred by heat conduction or by radiation, said point also being more sensitive to influences from organic and/or inorganic compounds than a sympathetic tone-neutral point.

Any sympathetic tone-neutral point can be used with any sympathetic tone-dependent point. The use of sets of a sympathetic tone-neutral point and a sympathetic tone-dependent point either anteriorly or posteriorly is preferred. As an example, it is preferable to use the sympathetic tone-neutral point anteriorly on the upper side of the clavicle in combination with the sympathetic tone-dependent points C.V. 17 or St 18 or Per 1 or preferable to use the sympathetic tone-neutral point TH 10-11 in combination with the sympathetic tone-dependent point TH 3-6.

The invention thus relates to a method of determining the sympathetic tone and/or the level of stress and/or the status of the warning system sensitivity in a subject, including the steps of: measuring an applied stimulation at a threshold value of the stimulation in one or more sympathetic tone-neutral points and measuring an applied stimulation at the same threshold value in one or more sympathetic tone-dependent points, or measuring an applied stimulation at a threshold value in one or more sympathetic tone-dependent points and optionally comparing said threshold value to a predetermined or pre-established calibration threshold value. Typically, the threshold value of said stimulation is the stimulation's nociception threshold value in the relevant point of stimulation.

According to a particular embodiment of the invention, an applied stimulation may be provided by an applied mechanical, thermal, electrical, radiation and/or chemical stimulus.

According to a particular embodiment of the invention a mechanical stimulus may be provided by an applied compressive force.

According to a particular embodiment of the invention a mechanical stimulus may be provided by an applied vibrating force.

According to a particular embodiment of the invention, a thermal stimulus may be provided by an applied heat or cold source.

According to a particular embodiment of the invention an electrical stimulus may be provided by an applied alternating current or an applied direct current.

According to a particular embodiment a radiation stimulus may be provided by means of an applied infrared, visible and/or ultraviolet light or combined spectra thereof, e.g. a laser, light-emitting diode, infrared, ultraviolet and/or white light source.

According to a particular embodiment of the invention a chemical stimulus may be provided by an applied organic and/or inorganic compound.

According to a particular embodiment of the invention, the determination of an applied stimulation at a threshold value of the stimulation may be carried out by means of a system for measuring the applied stimulation.

According to a particular embodiment of the invention, the measuring of the applied stimulation at a threshold value of the stimulation in a sympathetic tone-neutral point may be performed anteriorly on the upper side of the clavicle and/or posteriorly on the spinal column corresponding to TH 10-11 and/or on a finger and/or on a toe; in the latter two cases, the applied stimulation is preferably performed on the dorsal side of the finger/toe.

According to a particular embodiment of the invention, the measuring of the applied stimulation at a threshold value of the stimulation in a sympathetic tone-dependent point may be carried out at one or more locations on the skin which innervationally correspond to the nerve supply of the sympathetic nervous system to the heart, e.g. in one or more of the anterior points to which three locations are connected: C.V. 17 in the middle of the sternum, ST 18 between two ribs below the nipple and Per 1 between the nipple and the anterior axillary fold and posteriorly corresponding to TH 3-6 in the area between the shoulder blades, where the most sore of the said points is chosen.

According to a particular embodiment of the invention it relates to a method of quantitative and/or qualitative determination of sympathetic tone and/or level of stress in an animal, including human, and/or level of WSS and/or DRS, said method including:
a) storage of a calibration threshold value and a stimulation threshold value, the calibration threshold value being a quantitative measure of a nociception threshold value in a sympathetic tone-neutral point on or in the animal's body and the stimulation threshold value being a quantitative measure of a nociception threshold value in a sympathetic tone-dependent point on or in the animal's body, and subsequently;
b) calculation of an indication value of sympathetic tone and/or level of stress and/or level of WSS and/or DRS by comparing the stimulation threshold value with the calibration threshold value, whereby the indication value is a measure of the sympathetic tone and/or level of stress and/or level of WSS in the animal.

In an embodiment, the calibration threshold value and the stimulation threshold value are measured substantially simultaneously. The calibration threshold value may, however, also represent a historic mean value obtained on the basis of previous measurements or a predetermined value such as a constant which for instance may represent an average value of a number of different persons. In certain embodiments, the calibration threshold value is set to zero.

When using pre-established or predetermined calibration threshold values, this is primarily done in order to facilitate a user-friendly approach to the methods of the invention, since it will only be necessary to perform one single measurement in order to obtain a simple and readily accessible indication of the level of stress and/or sympathetic tone.

At any rate, the indication value of the sympathetic tone/level of stress is a preferably a mathematical combination of the calibration threshold value and the stimulation threshold value. This is to mean, that the read-out of the method is a value (or other alpha-numerical indication such as a colour code, a tone etc.) which is obtained by mathematically combining the stimulation threshold value and the calibration threshold value. Typically, the mathematical combination is a mathematical function (normally a monotonic function) of the ratio between the calibration threshold value and the stimulation threshold value (i.e. a function of the calibration threshold value divided by the stimulation threshold value). However, the mathematical combination may also be a function of the difference between the two values.

It should be noted that even though it is practical to do so according to the invention, these two threshold values need not be measured using the same unit of measure, and it is even conceivable (although not highly practical) that the two threshold values are threshold values for different types of stimulation.

In an embodiment nociception is induced by means of a exposure to compressive force, heat, cold, radiation, chemical stimulus or combinations thereof.

According to an embodiment, a significantly lower nociception threshold value in a sympathetic tone-dependent point than in a sympathetic tone-neutral point indicates that a person has WSS/DRS-related increased sympathetic tone.

The determined indication values of the sympathetic tone can be recorded either here-and-now or over a lengthy period of time. A particular embodiment thus relates to a method in which the indication value of the sympathetic tone is compared to at least one previously determined indication value of the sympathetic tone, said previous value indicating sympathetic tone at a earlier point in time.

Various uses of the method of the Invention

Since the methods referred to above are capable of providing a quantitative and/or qualitative measure of sympathetic tone and/or level of stress and/or level of WSS and/or DRS, it is possible to utilise the methods in connection with a large number of treatments (medical and non-medical) where the efficacy of the treatment is to some extent dependent on the sympathetic tone and/or level of stress in the patient being subjected to the particular treatment.

Numerous disease states are known to be associated with changes in sympathetic tone, which may either contribute directly to the pathogenesis of the disease (e.g., ischemic heart disease, hypertension) or be a consequence of the primary disease (e.g., pain, endocrinological disorders, psychological disorders). In either case, the method of the invention may be used to accurately monitor the influence of any pharmacological intervention prescribed to either treat the primary disease or to alleviate symptoms that are associated with major discomfort. To the patient and/or practitioner, this measure provides a measurable read-out that allows the patient to monitor efficacy of the pharmacological treatment and if necessary to effect a change of the therapy in order to provide an optimization thereof.

So, the present invention provides for a method for controlling the progress of a patient's therapeutic regimen (medicinal, surgical or by other means), wherein the efficacy and/or patient compliance of said regimen is dependent on sympathetic tone and/or stress level and/or level of WSS and/or DRS in said patient, comprising
i) determining one or more times during the course of the therapeutic regimen the sympathetic tone and/or level of stress and/or level of WSS and/or DRS in said patient, and
ii) adjusting the therapeutic regimen based on an integrated measure of the patient's benefit from the therapeutic regimen and determination is step i. Preferably, step i is performed by one of the methods for determining sympathetic tone and/or level of stress and/or level of WSS described herein.

The term “integrated measure of the patient's benefit from the therapeutic regimen and determination of level of stress/WSS and/or DRS” denotes a combination of information concerning the general and/or specific medical status of the patient, the patient's stress/resource balance and the stress/WSS/DRS level measurement disclosed herein together with information which gauges to what extent the patient has in fact followed or been subjected to the therapeutic regimen. In other words, the presently disclosed methods for determination of sympathetic tone and/or stress/WSS/DRS level are used together with more traditional means and measures for evaluating effect or compliance of a therapeutic regimen in order to arrive at a more accurate evaluation of said efficacy and/or compliance.

Examples of pharmacological compliance enhancement is expected during therapy with the following non-limiting group of therapeutic regimens: treatment with SSRI (Selective Serotonine Re-uptake Inhibitors), psychopharmalogical treatment of psychological, mental or behavorial disturbances, which are influenced by stress, including depression, other mood disorders, addiction, dependence disorder, neurosis, and suicidal behavorior, insulin-treatment in diabetes, nicotine substitution used as adjuvant therapy in smoking cessation, hormonal therapy in postmenopausal syndromes, hormone or other therapeutic means with respect to reproduction, fertility and miscarriage treatment, antiinflammatory therapy in acute and chronic inflammation, antiinfective therapy in infectious diseases, treatment of hypo- or hyperthyroid conditions, treatments with respect to dental care, treatment of diseases in heart, vessels, and kidney using cardiovascular drugs, treatment of ulcers, irritable bowel syndrome, malabsorption, nausea, and other symptoms using gastrointestinal drugs, pharmacological treatment with body weight lowering drugs, exercise programmes, relaxation programmes, diet programmes, counselling or coaching, stress-management programs, personal development programmes, personal performance programmes, and self-care programmes.

Notably, the use of the method of the invention in controlling treatment with SSRI is based on surprising findings made recently by the present inventors. In general, newly diagnosed female cancer patients exhibit a very high level of stress/WSS as determined by use of the presently disclosed method (as evidenced by a lowered threshold level for pain stimuli in sympathetic tone-dependent points), but it was observed that one single female patient, who differed from other examined female patients in that she received SSRI treatment, exhibited a significantly lower level of stress/WSS in spite of the fact that she was newly diagnosed with metastatic cancer. Further, the general condition of the female cancer patients included the presence of NWR, something which could not be found in the SSRI-treated woman.

As discussed above, it is also possible to utilise the methods for stress/WSS/DRS-determination of the present invention with a view to intervention. Thus, the invention also relates to a method for prevention of undesired or unproductive stress, the method comprising
a) determination of sympathetic tone and/or the stress/WSS/DRS level in a patient by means of the methods disclosed herein,
   and if the determination in step a indicates an elevated sympathetic tone and/or level of stress/WSS/DRS, then subjecting a sympathetic tone dependent point on or in the patient to a stimulation having a lower intensity than the stimulation threshold value for a period of time.

This particular intervention can e.g. be used to treat a patient which suffers heavily from stress and is in a situation where immediate treatment is necessary. However, it is also possible to use a more long-term treatment regimen in less critical cases, where the above method further comprises, after step a,
b) a determination of sympathetic tone and/or the stress level in a patient via the methods disclosed herein, and if this new determination does not indicate a less elevated sympathetic tone and/or level of stress, then subjecting a sympathetic tone dependent point to a stimulation having a lower intensity than the stimulation threshold value for a period of time which is different (preferably longer) from the period of time in step a, and

c) repeating step b until the determination indicates a less elevated sympathetic tone and/or level of stress than the determination in step a. Conveniently, and especially practical in cases where the intervention is not performed by a skilled practitioner, the subjection of the sympathetic tone dependent point to the lower stimulation intensity may be controlled by indicating a correct stimulation intensity by means of a visible or audible indication—in this way, it is avoided that the stimulation becomes insufficient or too forceful. Of course, this embodiment requires that a system or apparatus including means for sensing the intensity of the stimulation is used by either the patient himself or a practitioner.

This embodiment may include that the subjection of the sympathetic tone dependent point to the lower stimulation intensity is controlled by a closed loop system capable of stimulating a polymodal sensor cell and capable of measuring afferent impulses from said polymodal sensor cell and/or efferent motor cells related to the muscular defense reflex reations; for details on such systems, cf. below.

It is also possible to use the present invention prognostically in a method comprising
1) determining the sympathetic tone and/or level of stress in the patient, and subsequently
2) providing a prognosis for the patient with respect to the disease by incorporating in the determination of the prognosis the result of the determination in step 1, a determination in step 1 indicating a low sympathetic tone and/or level of stress/WSS/DRS being indicative of a better prognosis than a determination in step 1 of a higher sympathetic tone and/or level of stress/WSS/DRS. Also here, it is preferred that the determination in step 1 is performed by methods disclosed herein. Accordingly, it is also possible to use this method prophylactically, since it becomes possible to intervene in patients which have a poor prognosis and/or are at risk for developing either a specific disease condition and/or disease in general

The disease is preferably selected from the group consisting of an acute, subacute or chronic inflammatory condition;
a condition in which immunological reactions cause harm to human organs or in which insufficient function of the immunological system cause impaired function and/or disease;
an acute, subacute and chronic infectious disease;
a cardiovascular disturbance, which is affected by sympathetic tone, such as circulatory shock, atheriosclerosis, thrombosis, an ischemic condition, infarction, cardiac arrhythmia, hypertension;

a neoplastic growth disturbance;
an aquired metabolic disturbance;
a poisining or physical damage due to mechanic, thermal, electrical or radiation energy;

a psychological, mental or behavioural disturbance, which are influenced by sympathetic tone, such as depression or other mood disorders, an addiction or dependence disorder of any kind, a neurosis, a suicidal behaviour, a sleep disturbance, fatigue, a stress-related complain of psychological and/or mental character;
a fertility decrease in both female and male;
a gynaecological disturbance, which is influenced by sympathetic tone, such as premenstrual syndrome, dysmenorhea, menopause problems, hyperemesis gravidarum, preeclampsia and eclampsia, premature labor, situs invertus, induction of Labor, postpartum hemorhage;
an otolaryngological disturbance, which is influenced by sympathetic tone, such as tinnitus and presbyacusis;
a dermatological disturbance, which is influenced by sympathetic tone such as pruritus;
a gastrointestinal disease with stress-sensitive clinical signs and symptoms, such as gastric and duodenal ulcer, irritable bowel syndrome, malabsorption, diarrhea, constipation, nausea, and vomiting;
a neurological disturbance, such as tension headache, migraine, concussion, Parkinson's disease, Alzheimer's disease, intracranial traumas, and neuropathies an endocrinological disorder, such as diabetes, hypothyroidism, hyperthyroidism, an adrenocortical disorder, adrenomedullary disorder, a hypothamic disorders, a pituitary disorder, and polycystic ovary syndrome;
a allergy, such as one with reactions in skin, bronchi, and the gastrointestinal tract;
a pulmonary diseases with impaired gas exchange, such as bronchitis and emphysema;
a disease in joints and bone, such as, acute or chronic arthritis and osteoporosis;
a disorder related to changes in body weight composition, such as obesity, weight loss, cachexia;
a sodium and water-retaining disease state, such as heart failure, kidney failure, liver failure; and
pain.

One especially interesting embodiment of the invention is based on the finding that the method of the invention for measuring stress/WSS/DRS and/or sympathetic tone correlates well with known measurements for myocardial oxygen consumption. Thus, the invention provides a method for determining myocardial oxygen consumption in a subject, the method comprising a determination according to the method of the invention in the subject and determining the level of myocardial oxygen consumption based on a predetermined correlation to sympathetic tone measurements. This correlation has been shown to be significant in the sense that there is a significant correlation between measurements of the “Pressure Rate Product” (the product between systolic blood pressure and heart rate; cf. Opie L H. Normal and abnormal cardiac function: in Heart Disease. Braunwald E, Zippes D P & Libby P. (ed): W. B. Saunders 2001 (6th ed): 468-469; and Noble R E: Diagnosis of stress: Metabolism 2002; 51 (5): 37-39)) and the measurements of the invention; hence the correlation between myocardial oxygen consumption and sympathetic tone or WSS/DRS is preferably based on a pre-established correlation between sympathetic tone determinations and PRP (Pressure Rate Product) determinations.

PRP mirrors the heart's blood supply and thus work capacity. It also mirrors the degree of stress. PRP constitutes a practical index for the heart's oxygen uptake and thus reflects the heart's work. The heart's need for oxygen can be increased by the heart rate, wall stress and contractility in the heart muscle which are all conditions that can precede ischemia in a patient with ischemic heart disease (Opie L H. Normal and abnormal cardiac function: in Heart Disease. Braunwald E, Zippes D P & Libby P. (ed): W. B. Saunders 2001 (6th ed): 468-469.). Oxygen uptake is regulated by beta-adrenergic catecholamines. As such, it is a useful indicator of the heart's sympathetic activity and thus the degree of the heart's physiologic stress load (Noble R E: Diagnosis of stress: Metabolism 2002; 51 (5): 37-39).

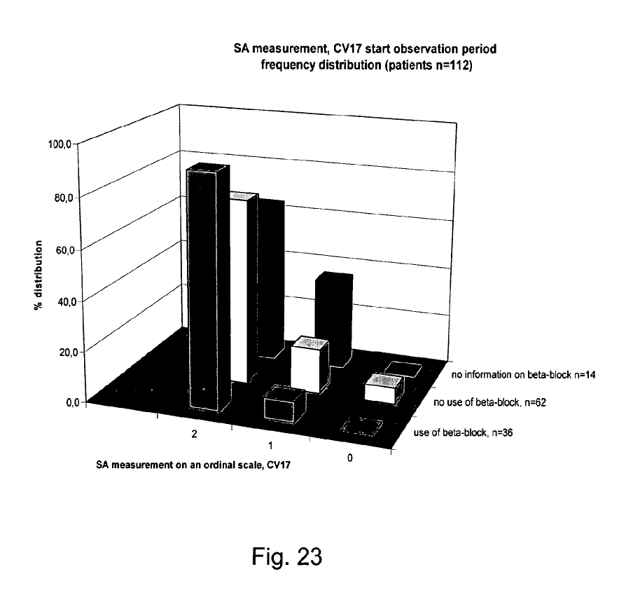

Another interesting embodiment of the invention is based on the finding that by using the method according to the invention, it is possible to distinguish between psychological stress and physical stress. It has thus been shown that the measurements obtained according to the invention (SA levels) do not always follow PRP (i.e. sympathetic tone of the heart). When the workload of the heart is changed due to changes in mental stress/psychological stress, the measurements obtained according to the invention follow the sympathetic tone of the heart. However, when the driver behind the workload i.e. the sympathetic tone of the heart is exclusively physical performance, the sympathetic drive on the heart changes concomitantly, as expected but the measurements obtained according to the invention do not (SA levels). Thus, in one aspect of the invention the measurements obtained according to the invention can be used to reflect the psychological level of stress (i.e. the level of WSS/DRS), rather than the sympathetic tone of the heart as such. It does reflect the sympathetic tone of the heart, when psychological stress is involved cf. the study shown in FIGS. 20, 21, 22 and 25. The result of the study shown in FIGS. 20, 21, 22 and 25 have subsequently been confirmed in another study with 112 consecutive patients with verified angina pectoris (ischemic heart disease) the results of which have been shown in FIG. 23. The tested group of patients is relevant, as more than 90% of these patients have elevated SA levels. Beta-blockage medication inhibits transformation in beta-adrenergic sympathetic receptors of the heart thus leading to a decrease in the sympathetic tone of the heart. The hypothesis to be tested in this study was that no change in SA measurements between two subgroups of patients (+/− beta-blockage treatment) would support the above mentioned findings, while a difference in tenderness would suggest that SA measures the sympathetic tone of the heart. In this study, the level of SA measurements was compared in two subgroups of this population: 1) 62 patients with no use of beta-blockage and 36 patients with daily use of beta-blockage (for the last 14 patients this information was not available). The results showed no significant difference in SA level at initial examination between the two groups of patients (p>0.1). The results thus support that the measurements obtained according to the invention (SA levels) measures psychological stress (=stress), rather than merely sympathetic tone of the heart.

The method of the invention also provides a surrogate measure for cardiac work capacity—this, however, requires that 2 measurements of the invention be combined, one for a “stressed state” and one for a relaxed state:

The method entails determining cardiac work capacity in a subject by determining sympathetic tone according to the method of the invention under stress conditions and during rest, respectively, and determining the cardiac work capacity of the subject based on a mathematical combination of sympathetic tone determinations under stress conditions and during rest. The mathematical combination is selected from a difference, a ratio, and any monotonic function thereof.

In diabetics, measurements of the level of glycated haemoglobin are used to determine the long-term efficacy of anti-diabetes treatment, but it is also accepted that such measurements provide information on the chronic stress-level of diabetic patients (where elevated values correlate with a chronic elevated stress level). However, it has been demonstrated that at similar levels of stress in a subject, the measured glucated haemoglobin correlates with the stress measurements of the invention, thus providing a method for indirectly determining the level of glycated haemoglobin in a diabetic subject by determining the sympathetic tone according to the method of the invention and subsequently determining the glycated haemoglobin level as a monotonic function of the sympathetic tone determination.

This determination is best performed when it is predetermined that the subject does not suffer from a high level of chronic stress, e.g. by means of the above-described methods or by means of a direct determination of the level of glycated haemoglobin.

This also opens for a variant of the above-described method for preventing/reducing stress, namely in a diabetic subject. The method comprises consolidating the measurements of stimulation threshold values with at least one measurement of glycated haemoglobin (since the latter can confirm that the subject is indeed suffering under a high chronic stress level, meaning that stress-reducing intervention is to be expected to have a beneficial effect). That the intervention is indeed necessary can be furthermore confirmed by studying a measure for transient stress (such as the above-mentioned PRP), which will not correlate with the measurement of the invention in the event the subject is suffering from chronic stress, whereas there is a strong and positive correlation between the measurement of the invention and PRP when the there is a low level of chronic stress.

The method of the invention opens for a number of possible self-monitoring or auto-monitoring schemes of variations in disease or treatment progress and these may be combined with rational intervention:

A patient with diabetes mellitus (DM) regularly measures blood sugar for estimation of need for insulin. However this currently entails the need for a blood test, which is not always available or practical. However, utilising the methods and systems of the present invention, e.g. where the system is incorporated in a mobile telephone, will allow the DM patient to keep track on his blood sugar in various environments. Since the blood sugar level decreases faster when the stress level is high, compared to when stress level is low, a stress measurement of the invention will provide a surrogate measurement which can indicate the need for more sugar or more insulin compared to the expected dosage. For instance, a DM patient may experience a number of stress indication measurements which indicates a deviation from the delicate balance between insulin intake, sugar intake and metabolic rate and with a build-in calculator attached to an alarm in the system (e.g. the cellular phone), it is possible to receive a proper warning that such a deviation has occurred in order to facilitate the correct course of action.

Also, a cancer patient receiving chemotherapy will benefit from an optimized effect of the therapy. In line with the above, a series of the measurements of the invention will be able to provide the information that the stress level has been elevated to an extent for such a length of time that decreased effect from the chemotherapy may be the consequence. Accordingly, when this information is provided by the system of the invention to the patient, he or she has the option to take proper action, i.e. actions which will decrease the stress level in order for the therapy to be maximally effective. Subsequently, the efficacy of this effort can be recorded as sufficient or not—until full effect is achieved.

Furthermore, a patient with ischemic heart disease (IHD) may use the similar information provided by a mobile track record of measure stress values. He or she may know that a certain day in the future a straining physical situation may occur and there is a substantial risk that the IHD may cause limitation of a definitive character. With the track record from the system of the invention and the continuous measurement in the period up to the event, the patient can act in a way that ensures a permanent low stress level (as measured by the method of the invention), which will enable that the physical task is performed without limitations. However, while in the middle of the event, the recording can provide the information if additional sympathetic tone reducing actions are needed. Such actions could for example be acupressure, rest or prophylatic anti-anginal medications.

One of the important findings leading to the present invention is that the methods of determining sympathetic tone and/or level of stress/WSS/DRS disclosed herein provide for an objective evaluation of stress-level. It thus becomes possible to utilise the methods as a quality control of other methods for determining stress in subjects.

Questionnaires for the use of measured level of stress is today regarded as evidence-based and used all over the world. However, in patients with persistent/chronic stress—such as for example women with breast cancer—the present inventors have found that the correlation between stress level measured by questionnaires and stress hormone in the blood is negative, indication that psychological neglect is present. It has, however, been found that the present technique has the ability to identify this negative correlation—and thus providing an objective (and non-invasive) control for the use of questionnaires in the measurement of clinical stress. This is supported by the test of 146 healthy persons, mentioned earlier, which revealed that half of the persons with a high level of persistent stress, experienced themselves as not being stressed or only stressed to a minor degree. Furthermore, is it known that stimuli with a potential threat are processed in the brain in the absence of conscious perception. In addition, scientific studies, testing the use of questinaires with respect to measurement of stress indicate the results are distorted due to 1) memory distortion, 2) impact of significant experiences and 3) distortion from the present state of stress.

Hence, the present invention relates to a method for determining whether an interview-based evaluation of stress level in a subject provides a true indication of stress, comprising,
a) in parallel to the interview, determining the sympathetic tone and/or level of stress/WSS/DRS in the patient by utilising any one of the methods disclosed herein, and
b) ascertaining whether the interview-based evaluation provides a result that correlates positively with the determination in step a, a positive correlation indicating that the interview-based evaluation provides a true indication.

The above thus allows for correct design of interview studies of stress.

Related to the above, it has been concluded by the inventors that the method of the invention is a convenient and non-invasive way of monitoring stress/WSS/DRS in a large number of situations. For instance, the method of the invention may be used for monitoring the level and/or nature of stress in a subject who is undergoing an investigative trial of a potentially stress-relevant nature. It thus becomes possible to examine whether a test treatment, which is aimed at reducing stress, actually has this effect, but it also becomes possible to determine whether a test treatment exhibits a tendency to induce or reduce stress, even in the event the focus of the treatment may be unrelated to stress.

Closed Loop Method for Controlling Sympathetic Tone/Stress

The present inventors have shown that the polymodal sensor cell affects the pain threshold during psychological stress (Ballegaard S, Karpatschof, Nyboe J, W. Trojaborg, Hansen A M, Magnusson G & Petersen B P. “Stress increases sensitivity of the biological warning system and defense reactions”; manuscript submitted for publication).

This opens for an embodiment of the present invention which utilises an implanted closed loop device for controlling sympathetic tone and/or stress level:

A small intracutaneous or subcutaneous stimulation unit sends out impulses (typical electrical) with increasing intensity, and the pain threshold of the adjacent polymodal sensors can be electrophysiologically gauged as the afferent nerve fibers will be activated. The efferent motor nerve cells associated with the noxious withdrawal reflex may also be used. In this case, the firing threshold of these cells are gauged.

When the afferent firing threshold is low, and/or the efferent firing threshold is low—indicating that the psychological stress level is elevated, an efferent electrical stimulation will be initiated by the device, thus leading to efferent signal transmission to the central nervous system with the aim to modulate=decrease level of sympathetic activity.

This closed loop: 1) afferent impulse—2) processing of response to the impulse—3) efferent impulse with the aim to change the physiological state of the organism—and 4) control of the effect by repeated afferent impulses can be most useful in conditions associated with persistent stress, e.g. metabolic syndrome, hypertension, depression, diabetes mellitus or ischemic heart disease. Other possible indications are various cancers as well as conditions characterized by chronic pain.

The third part of the above described loop does exist already and is known from devices used in epidural spinal cord stimulation in patients with ischemic heart disease (Sanderson J E, Brooksby P, Waterhouse D, Palmer R B G, Neubauer K. “Epidural spinal electrical stimulation for severe angina: a study of its effects on symptoms, exercise tolerance and degree of ischaemia”. Eur Heart J 1992; 13: 628-633; De Jongste M J L, Haaksma J, Hautvast R W M, Hillege H L, Meyler P W J, Stall M J, Sanderson J E & Lie K I. “Effect of spinal cord stimulation on myocardial ischemia during daily life in patients with severe coronary artery disease”. Br Heart J 1994; 71: 413-418; Landsherre C D, Mannheimer C, Habets A, Guilaume M, Bourgeois I, Augustinsson L E, Eliasson T, Lamotte D; Kulbertus H & Rigo P. “Effect of spinal cord stimulation on regional myocardial perfusion assessed by positron emission tomography”. Am J Cardiol 1992; 69: 1143-1149.

Hence, an important embodiment of the invention relates to a method for reducing the level of sympathetic tone and/or the level of stress and/or pain perception in a subject in need thereof, the method comprising
a) stimulating polymodal sensor cell(s) and/or efferent motor nerve cells in the subject so as to gauge the firing threshold, where the stimulation preferably is by means of electrical stimuli;
b) monitoring afferent impulses originating from the polymodal sensor cell(s) and/or the efferent motor nerve cell in response to the stimuation in step a);
c) if the monitoring in step b) reveals a lowered threshold for firing, which is indicative of elevated psychological stress/WSS/DRS, stimulating efferent nerve fibres to change the physiological state of the subject, and
d) repeating steps a)-c).

In this embodiment of the invention, it is preferred that stimulation of the efferent nerve fibres in step c) involves epidural spinal cord stimulation.

A related embodiment is a closed loop device for controlled stimulation of efferent nerve fibres, comprising
means for applying electrical stimuli to polymodal sensor cells and/or efferent motor nerve cells,
means for measuring afferent impulses originating from the polymodal sensor cells and/or the efferent motor nerve cells;
means for comparing measured afferent impulses with standard values;
means for applying electrical stimuli to efferent nerve fibres; and
means for controlling the application of electrical stimuli to efferent nerve fibres as a response to the comparison between the afferent impulses with the standard values.

The advances of the new concept is:
Very low intensitity of afferent stimulation is needed as the stimulation site is close to the polymodal nerve cell/efferent motor nerve cell
The intensity of the efferent stimulation needs to be low as well, for the same reason
The patient may or may not be involved—depending of step two is done automatically or with the conscious help for the person to indicate pain threshold—for example by pressing a bottum
As stimulation can be performed often, the needed adjustment of the physiological state with the respect to the individual stimulation can be minimized.

Alternative Method for Determination of Stress/Sympathetic Tone

As will appear from Example 12, the method for determining sympathetic tone/stress/WSS/DRS level by use of measurements in sympathetic tone-dependent points on or in the body has been found to correlate with the presence of noxious withdrawal reflex (NWR)—in brief an increase in the stress-level/sympathetic tone is accompanied by an increase in the presence of NWR.

This opens for the possibility to use NWR as a means for convenient and fast determination of the presence or absence of increased level of stress/sympathetic tone in an animal by determining the presence in the animal of a noxious withdrawal reflex (NWR) in response to a stimulus, said stimulus being one which does not elicit an NWR in a majority of individual animals in population having a normal sympathetic tone or not being stressed but which does elicit an NWR in a majority of individual animals in a population having increased sympathetic tone or high level of stress,

where the observation of an NWR in response to said stimulus is an indication that said animal has an increased sympathetic tone and/or level of stress.

The stimulus may be a stimulus which induces mild pain, but can also be any stimulus of the senses (a visible or audible stimulus, e.g.) which has been demonstrated to provoke an NWR in a majority of stressed animals but not in a majority of non-stressed animals.

One advantage of using NWR (which phylogenetically is a very well-conserved property), is that it can provide a fast qualitative answer to the question: “is the observed animal in a stressed condition?” The answer to this question is not only relevant when studying human subjects, but equally well when studying experimental animals, i.a. because stress in such experimental animals will be able to influence the reliability of scientific experiments performed on the animals—in more simple terms: a scientific study where the animals exhibits varying degrees of stress/WSS/DRS or sympathetic tone will be more likely to produce variation of result between animals and the consequence is that the study will require a larger amount of animals in order to demonstrate any differences effect. This means that if it is possible to screen the animals so as to exclude those that are (severely) stressed, the number of animals used in the study could be reduced. In this context, it would be convenient to observe such animals for the presence or absence of NWR—this can be done by regularly exposing the animals to a stimulus which predominantly will elicit NWR in stressed animals.

Of course, human subjects may also be “standardized” according to this principle, e.g. in clinical or pre-clinical trial settings where measurements of physiological effects are relevant. So, devising a simple putatively NWR-inducing test and observing whether or not the human subject in question reacts with an NWR will allow a objective sorting of human subjects which undergo scientific trials.

Likewise, it will be possible to provide a reliable gauging of animal welfare among domesticated or farm animals and already at an early stage determine that such animals are in a stressed state, even before they develop stress-induced behaviour.

The surveillance for NWR can be conducted in any of a large number of ways. For a particular type of animal, a relevant NWR can be selected and a corresponding NWR-inducing stimulus can be chosen. The behaviour of the animals can then be monitored, e.g. by camera surveillance or by any other convenient way of continuously or regularly observing NWR in the animals.

Systems of the Invention

The invention also relates to a system for measuring the sympathetic tone in an animal, including a human being, said system including:
a) Memory means for storing a nociception calibration threshold value determined at a sympathetic tone-neutral point on or in the animal's body and for storing a nociception stimulation threshold value in a sympathetic tone-dependent point on or in the animal's body;
b) An electronic circuit programmed to data process the nociception calibration threshold value and the nociception stimulation threshold value so as to obtain the measurement.

In an embodiment, the system according to the invention may further include user-operated means for applying a discomfort-inducing stimulus to the surface of the animal's body and user-operated storage means adapted to store the nociception calibration threshold value resulting from a first user operation.

The system may further, or alternatively, include user-operated means for applying a discomfort-inducing stimulus to the surface of the animal's body and user-operated storage means adapted to store the nociception stimulation threshold value resulting from a second user operation.

The means for applying a discomfort-evoking stimulus may be contained in a first unit and said electronic circuit may be contained in another unit. For obtaining the necessary data transfer between the first and second units the units may for instance be provided with means for wireless communication. In a so-called “distributed system” the first unit may for instance be a hand-held unit, which the user easily can bring with him/her, while the other unit may be a central computer at a doctor or hospital, said computer collecting data from a number of different users which each has a hand-held unit. Optionally the computer may be placed in the home of the user. In a distributed system a mobile phone may advantageously be used as communications means for transferring data from the hand-held unit to the computer, the hand-held unit for instance wirelessly transmitting data to the computer via a conventional mobile phone signal. This signal may be forwarded by a mobile phone provider to the computer via the internet. Optionally the second unit may be formed of a programmed mobile phone for instance communicating with the first unit via Bluetooth™, in which case a system utilizing the computing strength and memory storage of the mobile phone is used instead of a distributed system.

In a second embodiment, the means for applying a discomfort-evoking stimulus and the said electronic circuit are integrated in one and the same apparatus.

In an embodiment, the means for applying a discomfort-provoking stimulus are adapted to apply a stimulus which is gradually increased, the storage means being adapted to store a stimulation level at a moment in time corresponding to the first and second user operation, respectively.

In an embodiment the invention relates to a system in which the applied discomfort-evoking stimulus includes exposure to compressive force, vibration, heat, cold, electricity, radiation, chemical stimulus or combinations thereof.

In a particular embodiment of the system, the applied discomfort-inducing stimulus is stopped at the time of the first or the second user operation.

The invention further relates to a system for applying and measuring a stimulation to determine the sympathetic tone, said system including a measuring unit and optionally a read-out unit displaying the applied stimulation.

According to a particular embodiment of the invention, the system includes a marker for marking the measuring points such that it can be established where the stimulation was applied.

According to a particular embodiment of the invention, the system is provided with a scale divided into at least two zones, particularly four zones, which each for instance relates to the above levels of stress 0, 1, 2 and 3.

According to a particular embodiment of the invention these zones may have different colours, patterns or other distinctive marks which make them distinguishable from each other.

In an embodiment the pressure may be applied by means of a pressure base or a clamp.

The contact face of the pressure base is resilient in a particular embodiment.

In further embodiment the pressure base contains a liquid, a gel and optionally gas-filled bubbles.

In a particular embodiment the contact face on the pressure base is less than 4 cm², preferably between 1 and 2 cm².

The system according to a particular embodiment of the invention includes a pressure base with a contact face adapted to exert an outer compressive force on the animal's body, a sensor for measuring the compressive force exerted by the pressure base on the body, an electronic circuit adapted to store a first measured compressive force and a second measured compressive force, respectively, and to calculate a read-out value as an expression of the ratio between the first measured compressive force and the second measured compressive force, an embodiment of the system also including a read-out unit for displaying the read-out value. If the first measured compressive force is the measuring performed in a sympathetic tone-neutral point and the second measured compressive force is the measuring performed in a sympathetic tone-dependent point, the level of activity of the sympathetic nervous system may be displayed as the read-out value.

The system according to a particular embodiment of the invention includes a vibration base with a vibration head adapted to exert an outer vibrating force on the animal's body, a sensor for measuring the vibrating force exerted by the vibration base on the body, an electronic circuit adapted to store a first measured vibrating force and a second measured vibrating force, respectively, and to calculate a read-out value as an expression of the ratio between the first measured vibrating force and the second measured vibrating force, an embodiment of the system also including a read-out unit for displaying the read-out value. If the first measured vibrating force is the measuring performed in a sympathetic tone-neutral point and the second measured vibrating force is the measuring performed in a sympathetic tone-dependent point, the level of activity of the sympathetic nervous system may be displayed as the read-out value.

The system according to a particular embodiment of the invention includes a heating base with a contact face adapted to apply heat on the animal's body, a sensor for measuring the temperature applied by the heating base on the body, an electronic circuit adapted to store a first measured temperature and a second measured temperature, respectively, and to calculate a read-out value as an expression of the ratio between the first measured temperature and the second measured temperature, an embodiment of the system also including a read-out unit for displaying the read-out value. If the first measured temperature is the measuring performed in a sympathetic tone-neutral point and the second measured temperature is the measuring performed in a sympathetic tone-dependent point, the level of activity of the sympathetic nervous system may be displayed as the read-out value.

The system according to a particular embodiment of the invention includes an electricity base with an electricity head adapted to exert an outer electric force on the animal's body, a sensor for measuring the electric force exerted by the electricity base on the body, an electronic circuit adapted to store a first measured electric force and a second measured electric force, respectively, and to calculate a read-out value as an expression of the ratio between the first measured electric force and the second measured electric force, an embodiment of the system also including a read-out unit for displaying the read-out value. If the first measured electric force is the measuring performed in a sympathetic tone-neutral point and the second measured electric force is the measuring performed in a sympathetic tone-dependent point, the level of activity of the sympathetic nervous system may be displayed as the read-out value.

The contact face of the pressure base may be resilient. As a result a more accurate measurement is obtained, the contact face being adaptable to uneven areas on the body and provides a uniform pressure. The pressure thus corresponds to the applied force divided by the area of the contact face.

The pressure base may contain a liquid, a gel and optionally gas-filled bubbles, whereby a particularly snug fit to the surface of the body is obtained in the measuring point.

According to an embodiment the area of contact face on the pressure base may be less than 4 cm², such as between 1 and 2 cm², or less than 1 cm², such as between 0.5 and 1 cm².

The sensor may include a piezoresistive force sensor.

The vibration base may comprise an essentially spherical or hemispherical vibration head. In some embodiments, the diameter of the vibration head is less than 1 cm, such as between 0.05 mm and 0.5 mm, or less than 0.5 mm, such as between 1 mm and 4 mm.

Embodiment where a mechanical stimulus is provided by an applied vibrating force may comprise a magnetic actuator for the appliance of the vibrating force. The magnetic actuator may in some embodiments be arranged along a linear, possibly a straight, axis. The axis may, however, also be curved or otherwise ergonomically shaped e.g. in order to fit into a user's hand.

The magnetic actuator may comprise a plurality of sliding elements and a sliding bar. Each sliding element may include a bore or a cavity allowing it to be positioned on the sliding bar thereby enabling the sliding elements to slide along the sliding bar. The sliding elements and the sliding bar may be made of a non-magnetic metal, such as aluminium.

The sliding elements may be of a substantial circular shape, and each of the sliding elements may comprise a circumferential groove on its outer surface. The groove may be provided to support windings wound around the sliding elements. The windings may be made of a magnetic metal, such as copper.

The magnetic actuator may further comprise a plurality of magnets and separators, the separators being made of a material with a high magnetic permeability to ensure that they do not saturate.

In some embodiments, the sliding bar has a longitudinally extending bore or cavity, with the magnets and separators being alternatingly positioned inside the sliding bar. The sliding bar may further comprise two end pieces holding the magnets and separators in place inside the sliding bar. The magnets may be magnetized in the axial direction (in parallel with the pipe), and may be positioned with alternating pole directions.

The sliding bar may be designed to move inside the sliding elements together forming a hole, thus the sliding bar and/or the sliding elements may be coated with a friction reducing material, such as a lubrication.

A magnetic field perpendicular to the sliding bar may be created by the magnets. The sliding elements and sliding bar may be placed inside an iron casing, which may further maximize the magnetic field in the area of the windings.

The magnetic actuator may further be connected to a control unit enabling alternation of a current flowing in the windings. The connection may be by wire or alternatively be by wireless communication.

The magnetic actuator may create vibrations by alternating the current flowing in the windings. In one particular embodiment, the vibrations may be created in the following way: Current flowing in the windings is essentially perpendicular to the magnetic field and both are essentially in the plane perpendicular to the axis of the system. A force is thus created in parallel to the sliding bar. As the direction of the magnetic field is alternating for each sliding element and the sliding elements have alternating winding directions each sliding element/magnet pair will develop a force in the same direction. By changing the direction of the current the sliding bar will be pushed back and forth.

In a particular embodiment, the area of a contact face of the heating base is less than 4 cm², such as between 1 and 2 cm², or less than 1 cm², such as between 0.5 and 1 cm².

The heating base may be comprised in a heating actuator which may further comprise a control unit. The control unit may be positioned inside the heating actuator or may alternatively be connected to the heating actuator by a wired or wireless connection system.

The control unit may be provided to change the temperature of the contact face. A measurement may be started by pressing a start button on the control unit. When a build-in starting temperature is reached the measurements may be started. The build-in starting temperature may in some embodiments be varied by a user. A preferred starting temperature is 35° C. The measurements may automatically be started when a user presses the contact face towards the body.

The control unit may increase the temperature of the contact face until a build-in maximum temperature is reached or until a user interrupts the upward temperature movement. The user may interrupt the upward temperature movement, when his/her pain limit is reached. The control unit may record the temperature at which the measurement is interrupted.

The contact face may be cooled by natural cooling. In an alternative embodiment, a cooling element may be build-in the heating actuator.

The electricity base may comprise an essentially spherical or hemispherical electricity head. In some embodiments, the diameter of the electricity head is less than 1 cm, such as between 0.05 mm and 0.5 mm, or less than 0.5 mm, such as between 1 mm and 4 mm.

Embodiment where an electrical stimulus is provided by an applied electric force may comprise an electrical actuator for the appliance. These embodiments may further comprise a control unit enabling current flow in the electricity head.

In some embodiments, the control unit enables changing of current from alternating current to direct current and vice versa. In alternative embodiment, only alternating current or direct current is enabled.

In embodiment using direct current, the control unit may be provided to change the current flow in the electricity head. A measurement may be started by pressing a start button on the control unit. The control unit may comprise a build-in starting level for the current flow. The build-in starting level may in some embodiments be varied by a user. The measurements may automatically be started when a user presses the electricity head towards the body.

The control unit may increase the direct current flow in the electricity head until a build-in maximum flow is reached or until a user interrupts the increasing current flow. The user may interrupt the increasing current flow, when his/her pain limit is reached. The control unit may record the current flow at which the measurement is interrupted.

In embodiment using alternating current, the control unit may be provided to change the frequency of the alternating current flow in the electricity head. A measurement may be started by pressing a start button on the control unit. The control unit may comprise a build-in starting level for the frequency. The build-in starting level may in some embodiments be varied by a user. The measurements may automatically be started when a user presses the electricity head towards the body.

The control unit may increase the frequency of the alternating current flow in the electricity head until a build-in maximum frequency is reached or until a user interrupts the increasing frequency. The user may interrupt the increasing current flow, when his/her pain limit is reached. The control unit may record the frequency at which the measurement is interrupted.

In some embodiments, alternating current and direct current may be combined.

The electrical actuator may in some embodiments further be used to locate the point to stimulate.

The system may be integrated in an apparatus preferably being hand-held and power-supplied by one or more batteries. As a result the user may bring the apparatus along on travels.

The read-out unit may comprise an electronic display.

The electronic circuit may be adapted to determine the read-out value as one of a number, e.g. four, discrete read-out values (0, 1, 2, 3), the ratio between the first measured value and the second measured value being rounded off to or allocated a discrete read-out value (0, 1, 2, 3) displayed on the read-out unit.

The discrete read-out value (0, 1, 2, 3) may be non-proportional to the ratio between the first measured value and the second measured value. The apparatus may thus be accommodated to a lacking proportionality or linearity between the measuring results and the level of activity of the sympathetic nervous system. The level 0 may thus correspond to the second measured value being 80% or more of the first measured value, level 1 may correspond to the second measured value being 55-80% of the first measured value, level 2 may correspond to the second measured value being 30-55% of the first measured value, and level 3 may correspond to the second measured value being 30% or less than the first measured value.

The electronic circuit may be adapted to calculate the first measured value as an average of a number of measured values and calculate the second measured value as an average of a number of measured values. A more reliable measurement of the person's stress level is thus obtained, the error indication of the read-out value caused by measurement uncertainty at the individual measurements being reduced.

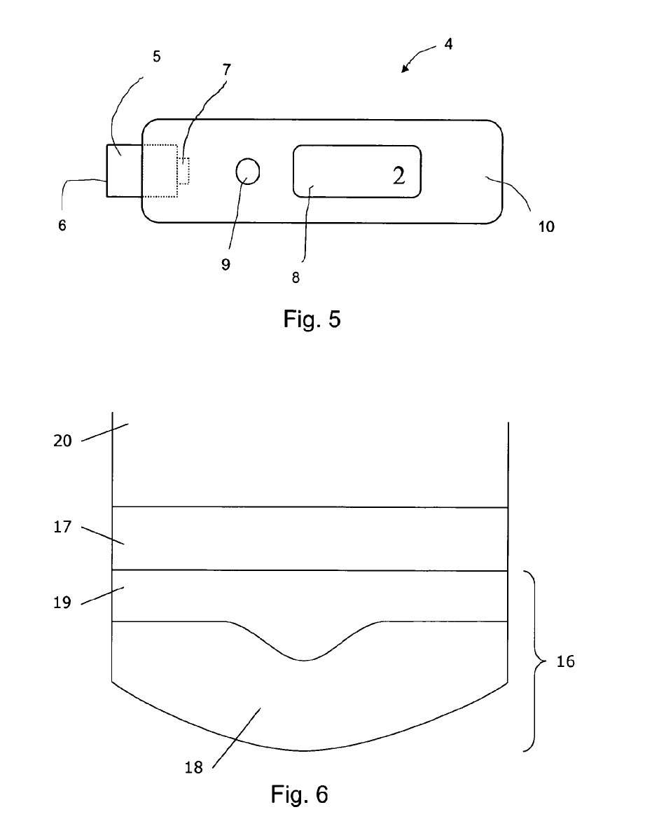

FIG. 5 shows a system according to the invention, the system being shown as integrated in one and the same apparatus for reason of clarity. The apparatus is hand-held and includes a housing 10, an electronic display 8, a control button 9 and a pressure base 5 extending from one end of the housing 7. The free end of the pressure base 5 has a contact face 6. Inside the apparatus the pressure base 5 abuts a force sensor or pressure sensor 7 connected to a not-shown electronic circuit. The electronic circuit is capable of storing the force or pressure measurements detected by the sensor 7. The circuit is further capable of making calculations and transmitting a read-out value to the electronic display 8. A not-shown battery supplies the circuit with power.

In use, the person holds the apparatus in his/her hand and exerts an increasing pressure on a sympathetic tone-neutral point on the body until the threshold value of discomfort has been reached. The electronic circuit records the maximum compressive force detected by the sensor. The person pushes the control button 9 and then exerts an increasing pressure on a sympathetic tone-dependent point on the body until the threshold value of discomfort is reached. The electronic circuit records the maximum compressive force. When the control button 9 has been pushed, the circuit calculates a read-out value as an expression of the ratio between the first measured compressive force and the second measured compressive force. In this example, the read-out value is 0, 1, 2 or 3, if the second measured compressive force is more than 80%, 55-80%, 30-55%, respectively, or less than 30% of the first compressive force. The apparatus may optionally be adapted to determine a mean value of a number of measurements of the first compressive force and a mean value of a number of measurements of the second compressive force, the read-out value being determined on the basis of these mean values. FIG. 5 shows a measuring in which the activity level of the sympathetic nervous system is 2 corresponding to the compressive force on the sympathetic tone-dependent point at discomfort being between 30% and 55% of the compressive force on the sympathetic tone-neutral point.

FIG. 6 illustrates a detail of a contact face 16 for applying a pressure force to a human's body. The contact face 16 includes a relatively soft convex member 18 made from a resilient material, such as a rubber material. The member 18 constitutes a distal end of a device embodying the system of the present invention. The relatively soft member 18 is supported by a relatively hard supporting member 19 made from, e.g., a plastics or rubber material or steel and defining a central protrusion into the relatively soft member 18. Accordingly, a centre portion of the contact face 16 is less resilient than a peripheral, outer portion thereof. The relatively soft member 18 may ensure that the contact face is capable of adapting its shape to uneven surfaces of the human's body. On the other hand, the relatively hard supporting member 19 may ensure that the force applied by the user is appropriately transferred to the body. The relatively hard supporting member 19 is supported by a pressure sensor 17, e.g. a piezoelectric sensor, mounted to or accommodated in a housing 20 of the device.

Whereas the contact face 16 is illustrated as a cylindrical member with a convex end portion in FIG. 6, it should be understood that alternative forms are envisaged. For example, it may be conical with a flat or convex end portion.

It will be appreciated that the system of the present invention may include means for applying a further stimulus to the surface of an animal's body, the further stimulus having a lower intensity than the stimulation threshold value, e.g. for purposes of treatment and/or stress relieve. For example, such means may be provided as a second, separate pressure base or they may be constituted by the pressure base also used for the purpose of measurement as described above. The first and second pressure bases may be provided at a free end of a hand-held unit, e.g. a pen-shaped unit. In one embodiment, the first pressure base is exchangeable with the second one, in which case the system of the invention preferably includes one pressure-base mounting spot only. In other embodiment, the system may accommodate the first and second pressure bases simultaneously, e.g. at opposite surface portions of a hand-held unit, for example at opposite ends thereof.

The contact face or pressure base of the device may be exchangeable, so as to allow the user or physician to choose a pressure base best suitable for a particular purpose or a particular patient. A plurality of pressure bases may be provided in a kit of elements together with e.g. a hand-held device incorporating other features of the system of the invention, e.g. pressure sensor, electronic control circuit, memory means and display.

A sound-emitting device may be provided for providing an acoustic signal to the user indicative of application of a desired force during treatment or stress relieve. Alternatively, an optical, vibratory or other signal emitter may be provided. The sound-emitting device is preferably connected to an electronic control circuit of the device, which receives input from the pressure sensor 7 or 17, so that a characteristic of the sound may be varied in dependence of the applied force. For example, the sound level, frequency, or the duration of intervals of silence between sound fragments or the duration of the sound fragments may be varied. In one embodiment, no sound is emitted, when the user applies a force, which is too low, whereas a sound having a first characteristic, e.g. a low frequency sound signal is applied, when the applied force is appropriate. If the force applied exceeds a predetermined threshold level, a high frequency alarm signal is emitted. Other embodiments are envisaged, including embodiments providing optical signals in a display of the device or by means of light-emitting diodes.

Example of Mechanical Stimulation using Pressure

An embodiment of the apparatus according to the present invention is comprised in a pen-shaped housing 4 (see FIG. 5) and is switched on by activating the push button 9 on the pen. Measurements are performed by pressing the rubber pad 5, placed on one end of the pen, against a pressure sensitive point on the patient. The pen automatically switches off when no further measurements are made within 1 minute.

The pen 4 measures the mechanical force acting on the rubber pad. The measurement cycle is started when the force rises above approximately 500 grams, and is stopped when the force drops beneath approximately 250 grams. During the measurement cycle the measurement value of the highest force is stored in the memory of the embedded computer.

After each measurement cycle the computer calculates a stress factor, which is shown in the display 8. This showing remains until a new measurement cycle is started—or the system is switched off by the time limit.

Measurement Principle and Linearization

The force sensor inside the pen-shaped housing of the system is a pressure sensitive ohmic resistor. At no force the resistance is relatively high. For increasing force the sensor resistance decreases, the reciprocal resistance being almost proportional to the force.

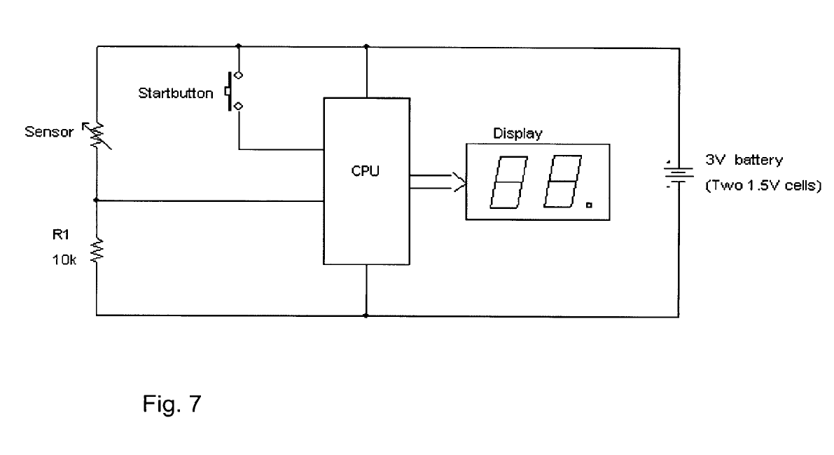

As seen in FIG. 7, the sensor is electrically in series with a fixed resistor R1, and this series connection is connected across the battery in the system. The voltage over resistor R1 is therefore a function of the force acting on the sensor. This voltage rises, when the force rises, but the relationship is non-linear.

The voltage at the top point of R1 is fed to an analogue input port of the computer, transforming the measurement voltage to a number between 0 and 1023 in the built-in AD-converter. As the battery voltage is used as reference voltage for the AD-converter, the measurement is not influenced by changes of the battery voltage.

The non-linear relationship between the force and the measurement value is changed to a corresponding linear relationship using the following algorithm:

corrected value=measurement value*correction factor/(correction factor−measurement value)

The correction factor is usually not a predetermined one. Its value is determined during an initial adjustment procedure.

Calculating the Stress Factor

During calibration of the system, the linearized value for 15 kg force is stored as a force reference value called “ref”. The linearized value of the maximum force during the latest measurement cycle is stored in the memory as a variable called “correctmax”. Next, the “correctmax” is transformed to a percentage of the reference force:

percent=100*correctmax/ref

Finally, the 7-segment display shows a stress factor, truncated to 2 decimal digits:

display=200−100*log₁₀(percent)

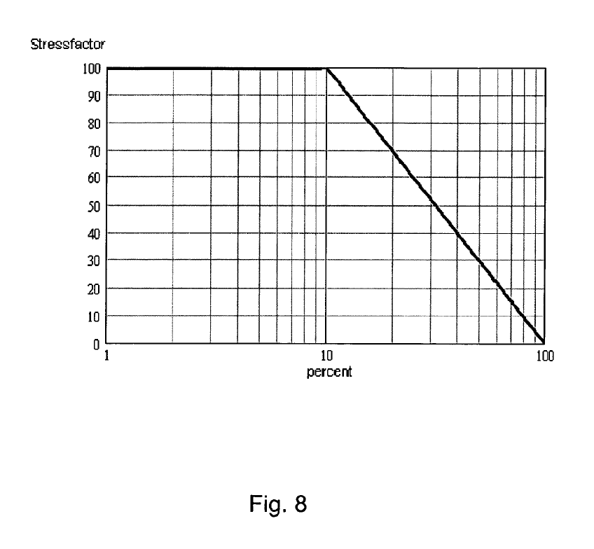

The stress factor as a function of “percent” is shown as a graph in FIG. 8.

Software Structure

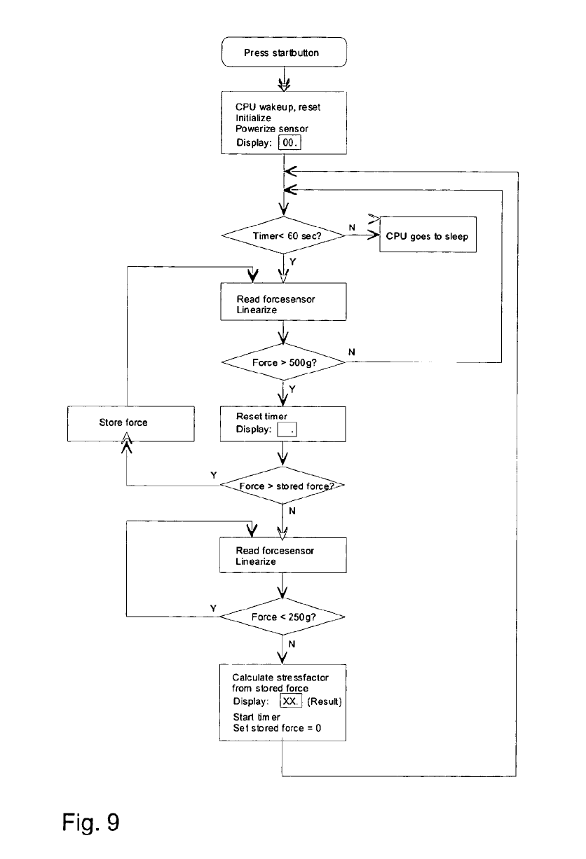

The basic principle for the data treatment is seen FIG. 9, showing a flowchart for the main function, which should be directly readable without further explanation. Side functions such as the surveillance of the battery voltage, generation of a beep sound when the sensor is overloaded (force >15 kg) etc. is not shown.

The processor cycles continuously with high speed through a main loop, alternating the attention between:
Data treatment: collecting analogue data, converting data to digital values, doing mathematic calculations etc., and
Controlling light in the segments of the LED display, one digit at a time.

Only about 20% of the cycle time is used for the data treatment. The remaining time is used for controlling the display.

Example of Mechanical Stimulation using Vibration

The mechanical shaping of the actuator is based on the pressure actuator described above. The rubber plug is replaced with a vibration head. The dimension of the spherical or hemispherical head is approximately 2 mm. A connected microprocessor based control unit controls the amplitude and frequency of the vibration.

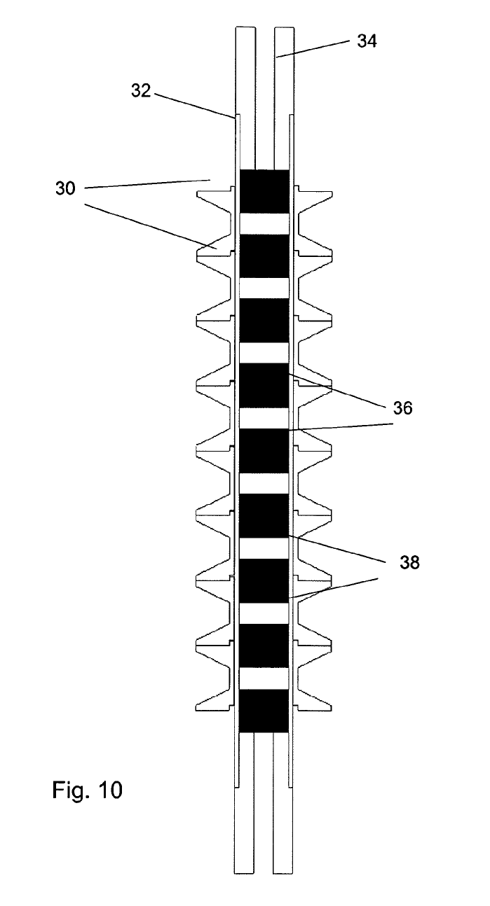

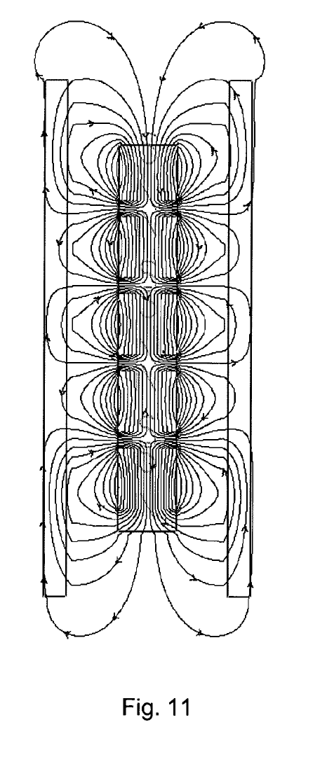

The mechanical part of the actuator is shown in cross section in FIG. 10. The protrusive parts are aluminium wheels 30 wound with 0.1 mm copper wire (not shown) in the groove. Eight of these are connected in parallel with alternating winding direction. The wheels are pushed together forming a hole with an inner diameter of 6 mm and a length of 48 mm. An aluminium pipe 32 with very walls is arranged in the hole. In each end of the pipe an end piece 34 holds the contents in place. The pipe is designed to be able to move inside the hole with minimal friction. The pipe is filled with strong rare earth (neodymium) magnets 36 separated by small iron discs 38. The magnets are magnetized in the axial direction (in parallel with the pipe) and placed with alternating pole directions.

The magnetic configuration creates a magnetic field shown in FIG. 11 for 5 magnets and 6 separators. Preferably, the separators are essentially made from iron with a high magnetic permeability to ensure that they do not saturate.

The pipe is placed inside the coils as shown in FIG. 10. Preferably, the separators are centred with respect to the wheels. Any displacement will weaken the pull created when current flows through the coils. The eight wheels are arranged inside an iron casing (not shown) that further maximizes the magnetic field in the area of the windings. The ideal situation would be if all field lines where perpendicular to the pipe in the area of the windings, as this would maximize the force that is developed when current flows. As seen from FIG. 11 this is, however, not the case in a practical implementation of the system.

An iron casing is made up of stacked laser-cut plates 40. End plates 41 close the encasing. A steel rod (not shown) is used for aligning the stacked plates perfectly using the small 2 mm holes cut in each plate. The iron encasing is arranged inside the extruded aluminium profile, and suitable end pieces close the device. Each end of the pipe is loaded with a spring that keeps the pipe centred with respect to the wheels.

The thickness of the pipe walls has been minimized taken production capabilities into account. As seen from the flux figure (FIG. 11), the magnetic field is stronger closer to the pipe. The pipe walls and the thickness of the wheels at the bottom of the grove take the winding away from the region with the strongest magnetic field.

The wheels and the rod should preferably be made from an essentially non-magnetic material.

Ideally the pipe and the wheels should be made from a non-conducting material. An alternating field will create eddy currents and induce losses in the aluminium.

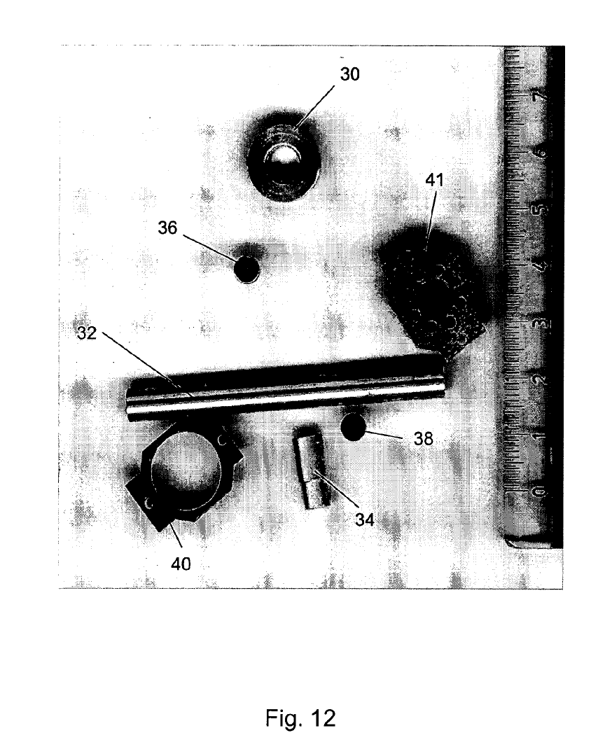

FIG. 12 shows different part of the actuator: aluminium wheels 30, aluminium pipe 32, end piece 34, rare earth magnet 36, iron disc 38, and parts of the iron casing 40 and 41.

Functional Description

Current flowing in the windings is essentially perpendicular to the magnetic field and both are in the plane essentially perpendicular to the axis of the device. A force is thus created in parallel to the axis. As the direction of the magnetic field is alternating for each wheel and the wheels have alternating winding directions, each wheel/magnet pair will develop a force in the same direction. The actuator has a limited stroke, as the effect described above will cease to work when the pipe is not centred with respect to the windings.

Actuator Driving and Software

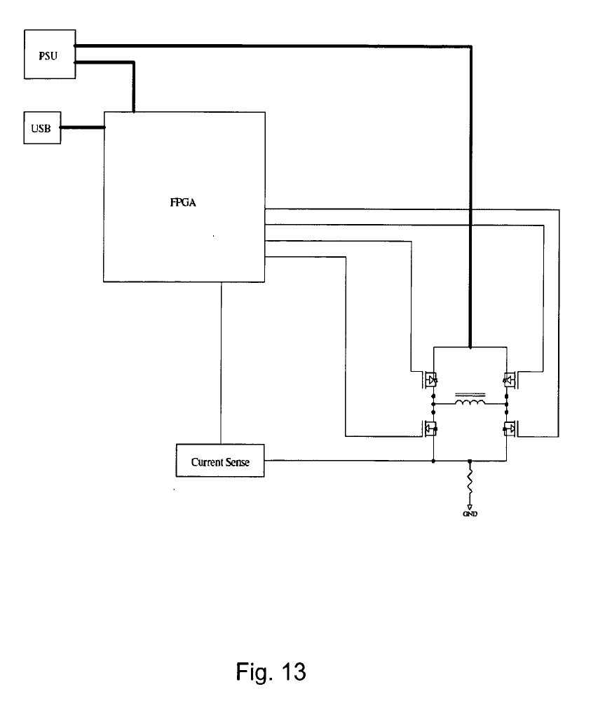

The actuator is driven using a simple H-bridge with current choppers limiting the steady state current in the windings (see FIG. 13). The FPGA contains an Altera NIOS based uP system equipped with an USB interface for connection to a host. By changing the direction of the current the pipe will be pushed back and forth.

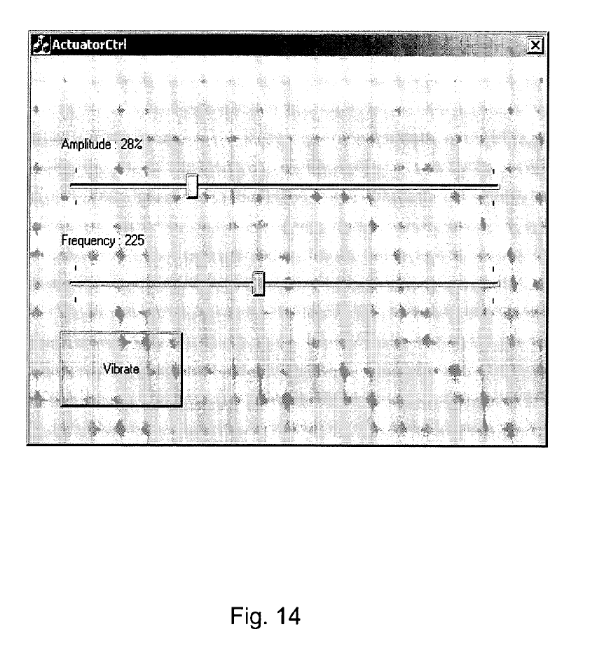

The stroke is adjusted by adjusting the current limit. This will affect the current flowing in the coils and thus the force exerted on the pipe. A small PC-based programme takes care of adjustment of frequency and stroke (see FIG. 14). The device connects as a simple HID USB device and receives simple commands via the programme. Vibration times out after 10 seconds in order to prevent overheating the coils.

According to a particular embodiment of the invention, the system may apply and measure a thermal stimulus, e.g. heat or cold.

Example of Thermal Stimulation

The mechanical shaping of the actuator is based on the above described pressure actuator. The rubber plug is replaced by a heating surface. The shape of the heating surface is almost identical to the shape of the pressure actuator.

Thus, the present embodiment includes:
a pen-shaped housing including hardware of the system,
a control-box, and
a battery charger unit.

The present embodiment includes a metal knob in the top end and a pushbutton named “HEAT” at the side of the pen-shaped housing. The control-box has a lighted start-button and a 2-line display in the lid. The pen is connected to the control-box with a thin cable. The control-box comprises a microprocessor which controls the temperature range and the temperature versus time.

A measurement is started by pressing the start button on the control-box. The pushbutton glows red indicating that the power is switched on, and the metal knob on the pen is heating up. The display writes: “Heating, wait”. When the pen after few seconds has reached the start temperature (35° C.), a build-in sounder beeps shortly and the display writes: “Ready”.

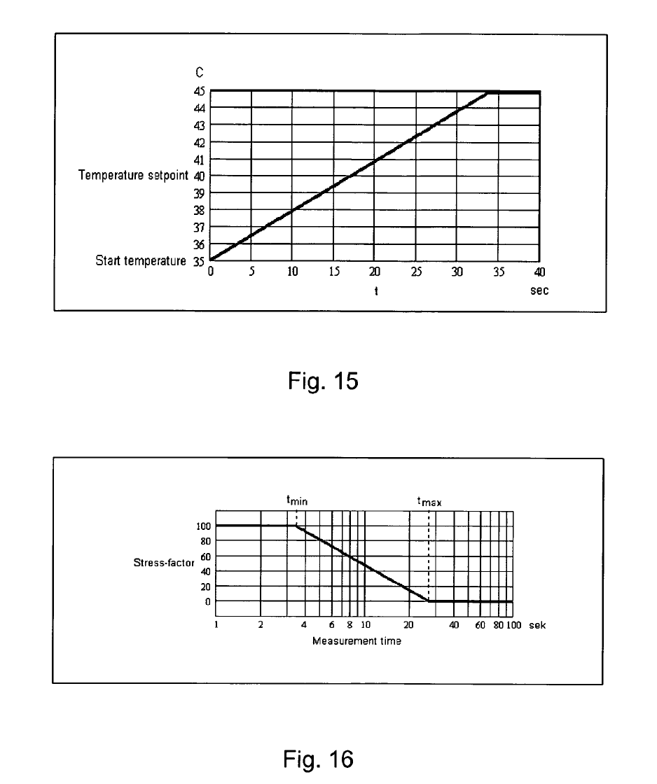

The metal knob is pressed against a sensitive point on the patient's body and the pushbutton “HEAT” on the pen is pressed. As long as the pushbutton is activated, the set-point for the temperature is gradually increased with a constant slope, eventually stopping at a maximum temperature (45° C.). The temperature set-point as a function of time is shown in FIG. 15. A control loop implemented in software makes the temperature of the metal knob following the temperature set-point. During this period, “Measuring” is appearing in the display.

When the patient reaches his/her pain limit the pushbutton on the pen should be released and the pen moved from the skin. The red light in the start-button on the control-box switches off, showing that the measurement has ended and the power in the pen is switched off.

The control-box then calculates and displays
Stress factor,
Elapsed measurement time, and
Max. temperature reached.

This showing is kept until either a new measurement is started, or the system reaches a power timeout. When the measurement period is over (the light in the start-button is off), the temperature of the metal knob decreases by natural cooling. If a new start is initialized before the start-temperature is reached, the start is postponed and the display writes: “Cooling, wait”. When the start-temperature again is reached, a beep sound is heard, “Ready” appears in the display.

Algorithm for Calculating the Stress Factor

The stress factor is a number between 0 and 100. The stress factor is logarithmic function of the elapsed measurement time t. When this is low, the stress factor is high and visa versa. The stress factor is calculated from the following equations, where t is the elapsed measurement time, tₘₐₓ and tₘᵢₙ are fixed, but adjustable maximum and minimum time values:

if t<tₘᵢₙ stress factor=100

if tₘᵢₙ≦t≦tₘₐₓ stress factor=100[log₁₀(tₘₐₓ)−log₁₀(t)]/[log₁₀(tₘₐₓ)−log₁₀(t_{min)]}

if t>tₘₐₓ stress factor=0

FIG. 16 shows the stress factor as a function of time.

Hardware Design

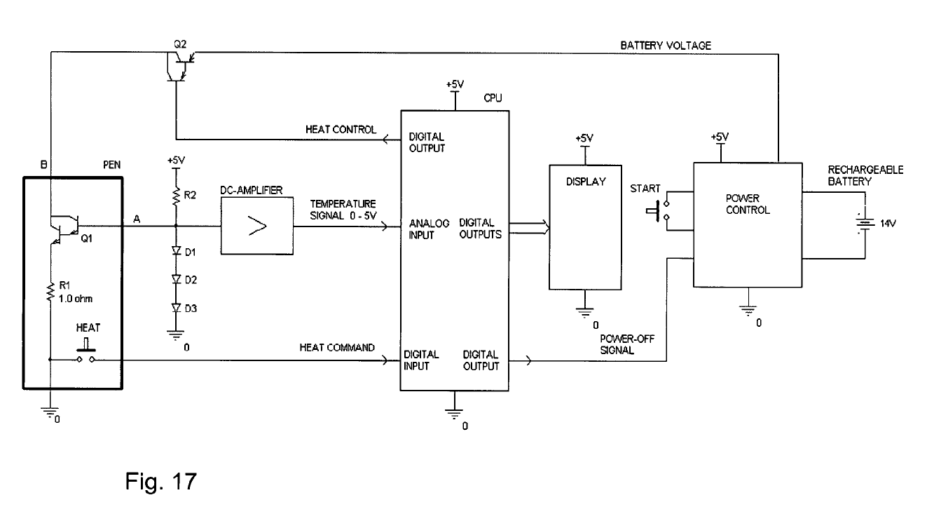

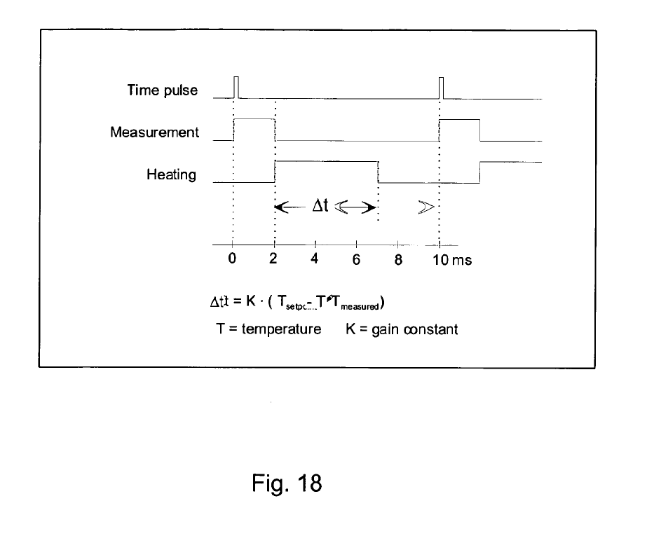

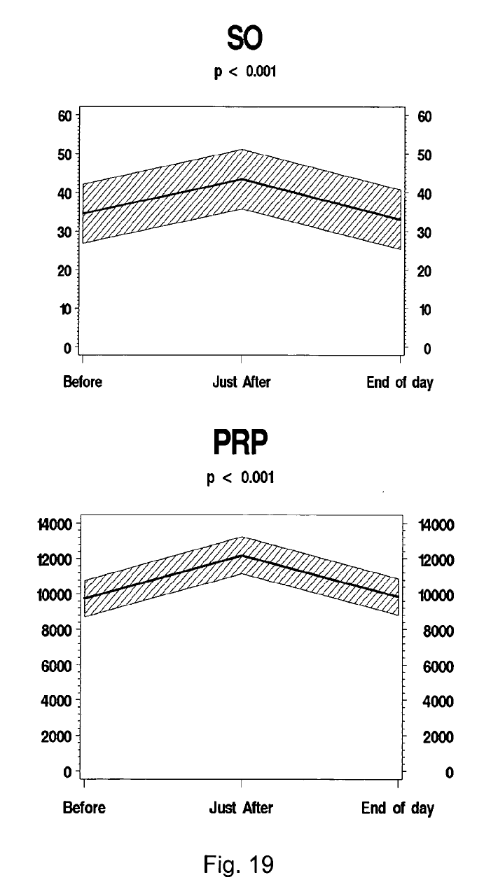

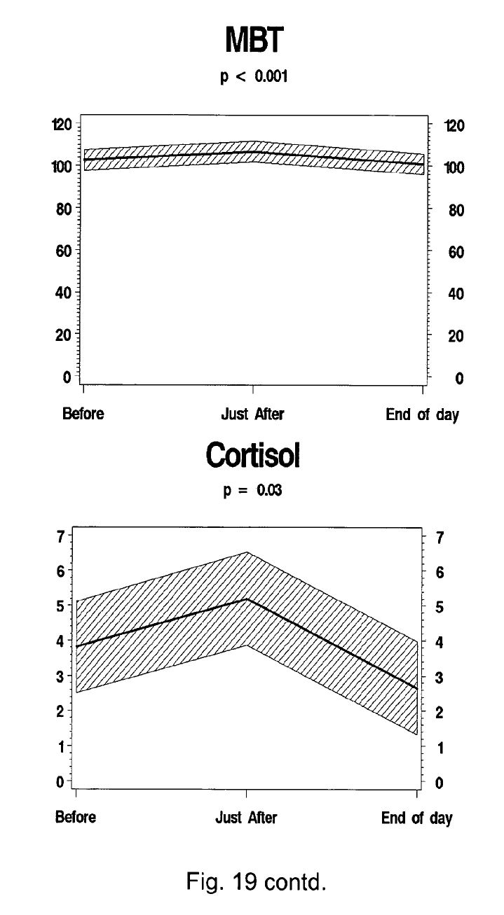

A simplified block diagram for the hardware design is shown in FIG. 17. The pen contains a Darlington power transistor Q1, a resistor R1 and a pushbutton named “HEAT”. The transistor has thermal contact with the metal knob on the pen. When pressed the pushbutton sends a heating command to the CPU. Q1 has two functions. In each sampling period, lasting 10 milliseconds, it acts a temperature sensor in the first 2 milliseconds. In the first part of the next 8 milliseconds it acts a heating element. This is shown in the timing diagram of FIG. 18.

In the measurement period of 2 milliseconds following happens, cf. the diagram of FIG. 17: The Darlington transistor Q2, placed in the control box, acts as a switch cutting the collector current of Q1 off. Through resistor R2 a low current (few milliamperes) flows through the Q1 basis-emitter-diodes and resistor R1 to ground. The voltage on point A is determined solely by the voltage drop V_{be} across the basis-emitter-diodes, which are temperature dependent. From room temperature, where V_{be} is about 1.4 V, this voltage decreases approximately 4 mV/° C., when the temperature increases. A DC amplifier amplifies the voltage on point A, giving 0-5 V output corresponding to 15-75° C. A 10-bit AD converter in the CPU receives this voltage and converts the temperature to a number from 0 to 1023 in the software.

In the heating period of max 8 milliseconds following happens: The Darlington transistor Q2 acts as an electronic switch and switches on, controlled by the CPU. The voltage on point B is close to the battery voltage V_{BAT}=14 V. The voltage on point A is close to 2.1 V, because it is now determined by the voltage divider comprising R2 and the diodes D1-D3, each having a knee-voltage of 0.7 V. As the basis-emitter voltage V_{be} is about 1.4 V, and R1=1Ω, the collector current I_{C} in Q1 is

I_{C}=(V_{A}−V_{be})/R1=(2.1−1.4)/1=0.7 A

When this current flows, the power delivered in Q1 is

P≈V_{BAT}·I_{C}=14 V·0.7 A=9.8 W

making a rapidly increasing temperature in Q1 and the attached metal knob.

As seen FIG. 18 this power is only delivered in the first part of the 8 milliseconds. The width of the power pulse At is modulated by the CPU by means of a software based proportional control loop:

Δt=K·(Tₛₑₜₚₒᵢₙₜ−T_{measured})

where K is an adjustable gain constant, Tₛₑₜₚₒᵢₙₜ the temperature set-point and T_{measured} the measured temperature.

The display is a 2-line 16-digit dot-matrix display with build-in controller and backlight, controlled by 14 lines from the CPU. Not shown in FIG. 17 are 5 pushbuttons, placed inside the control box and all connected to the CPU. These buttons named: Reset, Mode, Up, Down, and Save can be used for resetting the CPU and adjusting the following parameters: Tₛₜₐᵣₜ, Tₘₐₓ, slope, tₘᵢₙ, tₘₐₓ and the gain K.

The power is supplied from a build-in 14V rechargeable Li-Ion battery package. When the start-button is pressed a hardware based control logic switches the power on, also supplying 5 V for the CPU and other parts of the circuit. When 3 minutes has passed without any actions, the CPU sends a power cut-off signal to the power control circuit.

The battery charger is a commercial mains-supplied unit. For charging it is connected to the control box with a cable. For safety reasons the electronics is detached from the battery, when the plug of the charging cable is inserted the control box.

Software

The software structure is a so-called “Finite State Machine”, a structure commonly used in PLC's (Programmable Logic Controllers). This structure contains a closed loop of states, each containing one or more actions and one transition. A pointer runs through the total loop with high speed, activating the action(s) of one state only. This activation continues for many consecutive runs, until eventually the conditions for the belonging transition is fulfilled (true). In the following run another state is activated.

The conditions for the transitions are: Timer run-outs, counters reaching certain numbers, signals from other parts of the circuit being set or reset, pushbutton being pressed or released etc. (Further explanation, see “wikipedia.org” among others.)

Example of Electrical Stimulation

Electrical stimulation may be provided by the use of alternating current and/or direct current. As an example a commercially available actuator: Tao available from MibiTech ApS, Denmark, may be used, cf. WO 2004/062723, which is hereby incorporated by reference. The head of this electrical actuator is essentially spherical or ball-shaped having a diameter of about 2 mm. This electrical actuator provides electrical stimulus by an applied alternating current.

The electrical actuator has two functions: 1) the actuator may be used to locate the exact position to stimulate, and 2) the actuator may be use to carry of the stimulation. I.e. if the determination of sympathetic tone and/or stress level indicates an elevated sympathetic tone and/or stress level, a sympathetic tone dependent point may be subjected to an electrical stimulation having a lower intensity that the stimulation threshold for a period of time.

An advantage of this electrical actuator is that the ability to locate the point to stimulate is very well.

This electrical actuator may be described as electro-therapeutic device having first and second electrodes or probes for making electrical contact to the body of an individual. The device has voltage supplying means for supplying an alternating output voltage across the electrodes to pass an alternating current through the body of the individual, and the voltage supply means are adapted for controlling the frequency of the output voltage so that the output voltage frequency is automatically changing in time between a low frequency and a high frequency. The voltage supply means may be adapted for controlling the frequency of the output voltage so that the output voltage frequency is changing between a low frequency and a high frequency at regular time intervals. The actuator is further described in WO 2004/062723 “Electro-therapeutic device and method of electro-therapeutic threatment” (MibiTech ApS) which is hereby incorporated by reference.

According to a particular embodiment of the invention, said system may apply and measure a radiation, e.g. an infrared, visible and/or ultraviolet light or combined spectra thereof, provided as an example by means of a laser, light-emitting diode, infrared, ultraviolet and/or white light source.

According to a particular embodiment of the invention, said system may apply and measure a chemical stimulus, e.g. an organic or inorganic compound.

The invention further relates to the use of a system for applying and measuring a stimulation for determining the sympathetic tone including the steps of: measuring an applied stimulation at a threshold value of the stimulation in one or more sympathetic tone-neutral points and measuring an applied stimulation at the same threshold value of the stimulation in one or more sympathetic tone-dependent points.

According to a particular embodiment of the invention, a system is used for applying and measuring a stimulation for determining sympathetic tone, the measuring of an applied stimulation at a threshold value of the stimulation in one or more sympathetic tone-neutral points being performed anteriorly on the upper side of the clavicle and/or posteriorly on the spinal column corresponding to TH 10-11.

In an embodiment, the invention relates to use of a system for applying and measuring a stimulation for determining sympathetic tone, the measurement of an applied stimulation at a threshold value of the stimulation in one or more sympathetic tone-dependent points being performed at one or more locations on the skin which innervationally (i.e. relating to the nerve supply) correspond to the nerve supply of the sympathetic nervous system to the heart, e.g. in one or more of the points contained in the areas: C.V. 17 in the middle of the sternum, ST 18 between two ribs below the nipple and Per 1 between the nipple and the anterior axillary fold and on the back corresponding to TH 3-6 in the area between the shoulder blades, where the most sore of the said points is chosen.

According to a particular embodiment of the invention a system is used which is capable of applying and measuring a mechanical stimulus such as a compressive force.

According to a particular embodiment of the invention a system is used which is capable of applying and measuring a mechanical stimulus such as vibration.

According to a particular embodiment of the invention a system is used which is capable of applying and measuring a thermal stimulus such as heat or cold.

According to a particular embodiment of the invention a system is used which is capable of applying and measuring an electrical stimulus such as a alternating current or direct current.

According to a particular embodiment of the invention, a system is used which is capable of applying and measuring radiation, e.g. infrared, visible and/or ultraviolet light or combined spectra thereof, provided as an example in form of a laser, light-emitting diode, infrared, ultraviolet and/or white light source.

According to a particular embodiment of the invention a system is used which is capable of applying and measuring a chemical stimulus such as an organic or inorganic compound.

In a further aspect, the present invention provides a mobile telephone comprising or being connected to an embodiment of a system of the present invention. The system may be entirely integrated in the mobile telephone, thus including discomfort-evoking means as well as read-out means in the phone. Alternatively, only parts of the system may be integrated in the phone. For example, discomfort-evoking means may be included in a housing of the mobile phone, whereas read-out means may be provided at a remote location, at which appropriate means are provided for receiving data communicated from the phone. In one embodiment, a computer system may be provided at a hospital or a physician's practice, to which read-out values indicative of thresholds of pain, stress factor or other values are communicated. Such a computer system may also be provided in the patient's home. The read-out means may be connected to a display of the mobile telephone and/or to a loudspeaker of the phone capable of emitting a suitable audio signal, such as an artificial voice signal. For instance, a critical stress factor may be displayed as a warning on the display, or it may be indicated by an acoustic warning signal.

The mobile telephone may be adapted to remind the user of the phone to perform pain threshold measurements. For example, the phone may be programmed to emit a reminder signal at a given point in time, or it may be programmed to emit a reminder upon receipt of a predetermined wireless signal, included e.g. in an SMS or MMS message. Thus, a physician or other person may prompt the user of the mobile telephone to perform a nociception stimulation by means of the system of the present invention incorporated in the phone.

The phone may be programmed to log measurements performed by the user, i.e. to store values of several measurements, including e.g. nociception stimulation threshold values, stress factors or other values producable by the system of the invention. The time of the measurements may likewise be logged. Such logging may also be performed at a remote facility, to which the phone may connect via wireless communication. Alternatively, logged data may be transmitted from the phone to a computer system via a wired or wireless connection, such as BlueTooth™, when the phone is close to the computer system.

Activation of the stimulus-evoking means incorporated in the phone may be performed via the phone's keypad, via touch-screen, voice recognition or via a separate push button designated for that purpose.

The invention is further illustrated in the following examples:

### EXAMPLES

Unit in all measurements in the examples when measuring an applied compressive force by means of a manometer is British pounds (lbs)/cm², in the following referred to as lbs.

Units in all measurements in the examples when measuring a compressive force applied with a finger at the threshold value of the pressure sensitivity are: 0, +, ++, +++, where 0 is the applied compressive force at a threshold value of the pressure sensitivity in a sympathetic tone-neutral point, and where =, +, ++, +++ is the compressive force applied with a finger at the same threshold value of the pressure sensitivity in a sympathetic tone-dependent point, where 0 equals the applied compressive force in a sympathetic tone-neutral point, and +, ++, +++ is the relatively lower applied compressive force.

### Example 1

### Example 1a

The sympathetic tone of a person was determined in the following manner: By means of a manometer at a threshold value of the pressure sensitivity in the sympathetic tone-neutral point anteriorly on the upper side of the clavicle, the applied compressive force was measured to 13.8 lbs. Then the applied compressive force was measured to 13.0 lbs at the same threshold value of the pressure sensitivity in the sympathetic tone-dependent point C.V. 17. At the same threshold value of the pressure sensitivity in the sympathetic tone-dependent point the applied compressive force was thus 94% of the applied compressive force in the sympathetic tone-neutral point. According to the present invention this corresponds to Level 0 stress. The person then filled-in a questionnaire about the person's stress level, said questionnaire confirming that the person displayed no signs of clinical stress.

### Example 1b

The sympathetic tone of another person was determined in the following manner. By means of a manometer at a threshold value of the pressure sensitivity in the sympathetic tone-neutral point anteriorly on the upper side of the clavicle, the applied compressive force was measured to 14.3 lbs. Then the applied compressive force was measured to 11.0 lbs at the same threshold value of the pressure sensitivity in the sympathetic tone-dependent point C.V. 17. At the same threshold value of the pressure sensitivity in the sympathetic tone-dependent point the applied compressive force was thus 77% of the applied compressive force in the sympathetic tone-neutral point. According to the present invention this corresponds to Level 1 stress.

Example 1c

The sympathetic tone of a third person was determined in the following manner. By means of a manometer at a threshold value of the pressure sensitivity in the sympathetic tone-neutral point anteriorly on the upper side of the clavicle, the applied compressive force was measured to 10.0 lbs. Then the applied compressive force was measured to 7.0 lbs at the same threshold value of the pressure sensitivity in the sympathetic tone-dependent point Per 1. At the same threshold value of the pressure sensitivity in the sympathetic tone-dependent point the applied compressive force was thus 70% of the applied compressive force in the sympathetic tone-neutral point. According to the present invention this corresponds to Level 1 stress.

Example 1d

The sympathetic tone of a fourth person was determined in the following manner: By means of a manometer at a threshold value of the pressure sensitivity in the sympathetic tone-neutral point posteriorly on the spinal column corresponding to TH-10-11 the applied compressive force was measured to 24.0 lbs. The applied compressive force at the same threshold value of the pressure sensitivity in the sympathetic tone-dependent point posteriorly on the spinal column corresponding to TH 3-6 was measured to 22.5 lbs. At the same threshold of the pressure sensitivity in the sympathetic tone-dependent point, the applied compressive force was thus 94% of the applied compressive force in the sympathetic tone-neutral point. According to the present invention this corresponds to Level 0 stress. The person then filled-in a questionnaire about the person's stress level, said questionnaire confirming that the person displayed no signs of clinical stress.

### Example 2

### Example 2a

The sympathetic tone of a person was determined in the following manner: By means of a manometer at a threshold value of the pressure sensitivity in the sympathetic tone-neutral point anteriorly on the upper side of the clavicle, the applied compressive force was measured to 17 lbs. Then the applied compressive force was measured to 8.0 lbs at the same threshold value of the pressure sensitivity in the sympathetic tone-dependent point C.V. 17. At the same threshold value of the pressure sensitivity in the sympathetic tone-dependent point the applied compressive force was thus 47% of the applied compressive force in the sympathetic tone-neutral point. According to the present invention this corresponds to Level 2 stress.

### Example 2b

The sympathetic tone of another person was determined in the following manner. By means of a manometer at a threshold value of the pressure sensitivity in the sympathetic tone-neutral point anteriorly on the upper side of the clavicle, the applied compressive force was measured to 10.5 lbs. Then the applied compressive force was measured to 5.0 lbs at the same threshold value of the pressure sensitivity in the sympathetic tone-dependent point St. 18. At the same threshold value of the pressure sensitivity in the sympathetic tone-dependent point the applied compressive force was thus 48% of the applied compressive force in the sympathetic tone-neutral point. According to the present invention this corresponds to Level 2 stress.

### Example 2c

The sympathetic tone of another person was determined in the following manner. By means of a manometer at a threshold value of the pressure sensitivity in the sympathetic tone-neutral point anteriorly on the upper side of the clavicle, the applied compressive force was measured to 14.0 lbs. Then the applied compressive force was measured to 5.0 lbs at the same threshold value of the pressure sensitivity in the sympathetic tone-dependent point, Per 1, and to 5.5 lbs in the sympathetic tone-dependent point St. 18. At the same threshold value of the pressure sensitivity in the sympathetic tone-dependent points the applied compressive force was thus 36% and 39%, respectively, of the applied compressive force in the sympathetic tone-neutral point. According to the present invention this corresponds to Level 2 stress.

### Example 3

The sympathetic tone of a person was determined in the following manner: By means of a manometer at a threshold value of the pressure sensitivity in the sympathetic tone-neutral point anteriorly on the upper side of the clavicle, the applied compressive force was measured to 9.0 lbs. Then the applied compressive force was measured to 2.0 lbs at the same threshold value of the pressure sensitivity in the sympathetic tone-dependent point C.V. 17. At the same threshold value of the pressure sensitivity in the sympathetic tone-dependent point the applied compressive force was thus 22% of the applied compressive force in the sympathetic tone-neutral point. According to the present invention this corresponds to Level 3 stress.

### Example 4

The person mentioned in example 3 was given a personally calibrated system according to the invention for measuring an applied compressive force for determining the sympathetic tone, said system including a measuring device and a scale, which in this example was divided into four zones corresponding to the four levels of stress, said system displaying the applied compressive force and provided with a marker for marking of one or more measuring points. By using the supplied system according to the invention, the person was able to determine his/her sympathetic tone at any convenient time. As a result the person was subsequently able to determine the sympathetic tone by observing to which zone an applied compressive force corresponded at the threshold value of the pressure sensitivity in a sympathetic tone-dependent point. One zone corresponds to less than 30% (Level 3); another zone corresponds to between 30% and less than ⁵⁵% (Level 2); a third zone corresponds to between 55% and less than 80%; and a fourth zone corresponds to more than or equal to 80% of the applied compressive force at the threshold value of the pressure sensitivity in a sympathetic tone-neutral point.

### Example 5

The sympathetic tone of a person was determined in the following manner: By means of a manometer at a threshold value of the pressure sensitivity in the sympathetic tone-neutral point anteriorly on the upper side of the clavicle, the applied compressive force was measured to 9.0 lbs. Then the applied compressive force was measured to 2.0 lbs at the same threshold value of the pressure sensitivity in the sympathetic tone-dependent point C.V. 17. At the same threshold value of the pressure sensitivity in the sympathetic tone-dependent point the applied compressive force was thus 22% of the applied compressive force in the sympathetic tone-neutral point. According to the present invention this corresponds to Level 3 stress. The person then filled-in a questionnaire about the person's stress level, said questionnaire showing that that the person displayed symptoms of chronic accumulated stress.

The threshold value of the pressure sensitivity was determined to “+++” by using a finger.

Four weeks later—after suitable intervention—the same measurements were repeated for the person.

By means of a manometer at a threshold value of the pressure sensitivity in the sympathetic tone-neutral point anteriorly on the upper side of the clavicle, the applied compressive force was measured to 10.0 lbs. Then the applied compressive force was measured to 9.5 lbs at the same threshold value of the pressure sensitivity in the sympathetic tone-dependent point C.V. 17. At the same threshold value of the pressure sensitivity in the sympathetic tone-dependent point the applied compressive force was thus 95% of the applied compressive force in the sympathetic tone-neutral point. According to the present invention this corresponds to Level 0 stress. The threshold value of the pressure sensitivity was determined to “0” by using a finger.

At the same time, the person advised that the previously recorded clinical signs of stress had passed.

### Example 6

At a test with 250 randomly selected persons, the correlation between physiological stress and clinical stress was examined. The 250 randomly selected persons were told to fill out a questionnaire to ascertain whether they had experienced some specific situations within the last four weeks. There were 35 questions in total which represented different clinical signs of stress.

The persons were then instructed to examine themselves—in plenum—by initially identifying the upper side of the clavicle and there to register which intensity of an applied pressure was necessary to obtain the threshold value of the pressure sensitivity. With this as a starting point, the persons were instructed to locate C.V. 17 and based on the same procedure used on the upper side of the clavicle to determine the relative applied compressive force necessary to obtain the same threshold value of the pressure sensitivity on a four-point scale: 0, +, ++, +++.

All of the questionnaires were then collected and analysed.

The correlation between the applied compressive force to obtain the threshold value on the upper side of the clavicle in relation to C.V. 17 was significant (p<0.001), ie. the more stress symptoms experienced by the individual person within the last four weeks the less compressive force was to be applied to the thorax in the point C.V. 17 in relation to the upper side of the clavicle.

### Example 7

### Prognostic use of the Method/System

A completely healthy person, e.g. a musician or conductor, employs the method and system each morning to ensure a low measurement, which prognostically gives an optimum utilization of his/her resources when music is to be played/conducted later in the day.

If one morning the measurement is high, the measurement allow for initiation of stress-reducing activities, such as exercise/relaxation. When the activity has been completed, the person can measure whether this has had a sufficient effect, ie. a low measurement is obtained. If the desired goal has not been met, the procedure may be repeated.

### Example 8

### Daily Stimulation with a Preventive Effect

As in example 7, the system in this example, however, also being used for performing the following actions:

By means of the system such a strong continuous pressure is maintained in a sympathetic tone-dependent point that the pressure is felt without the stimulation causing pain. After 20-40 seconds the person registers that the subjacent soreness has decreased.

By means of the system this can be recorded as a 50% increased pain threshold; The pain threshold may thus increase from 40% to 60% of the threshold value in the sympathetic tone-neutral point.

Physiologically, this entails that the “stress phase” has passed and the restitution phase is activated.

This action may contribute to preventing negative stress.

### Example 9

### Ad Hoc Stimulation for Immediate Relief of Stress

As example 7, in this example, however, the user registers a high value and immediately performs the action as described in example 8. At a correctly performed action, the user will be able to register a likely 50% improvement in the measured value after 20-40 seconds.

### Example 10

### Measuring for Learning

As the method and system provide a here-and-now measurement of the stress level, i.e. the activity in the sympathetic nervous system, a person is able known his/her “morning value” and repeat the measurements during the day so as to identify specific situations affecting the stress level (e.g. a conversation, an order, a phone message, a task).

As the stress phase is activated within a few second and passes again within 20-40 seconds, the method and system provide completely new possibility for learning how different daily situations affect the stress level—both in negative and positive direction.

In the long view, the method is thus able to tell the person whether for instance a holiday has had the desired relaxing effect.

### Example 11

### Use of the Invention for Determining State of the Biological Warning System

Introduction—Biological Warning Systems and Warning System Sensitivity

Warning and defense systems have been an essential part of the survival strategy of living organisms throughout the entire evolution. In the early part of evolution, the bacteria, like Escherichia coli has developed a special sensory system, based on molecules on the surface of the bacteria, which monitor the chemical condition of the environment in respect of factors critical to their survival—and have developed a modulator system with the potential to induce stress tolerance. Further, the coiling reflex (a withdrawal reflex) in the amphioxus, a early ancestor to the vertebrates, represents the most primitive nervous reflex.

In animals the ability to detect a potentially tissue-damaging environmental stimulus is further developed into what is called the nociceptive system, which provides the organism with the information needed for an optimal response to adverse environmental conditions—and it may be accompanied by a reflex response such as withdrawal. The nociceptive system is based on a polymodal receptor, an undifferentiated nerve cell of identical nature throughout the evolutionary chain, from fish to higher vertebrates and humans. The receptor is stimulated by mechanical pressure, temperature and acidity. The sensitivity of the receptor may undergo different kinds of modulation:

One aspect is stress-induced analgesia, in which pain sensation is suppressed. This helps the injured/fighting animal to suppress general pain sensation in order to optimize fighting/fligthing capacity. A second aspect is stress-induced hyperalgesia, in which pain sensation is increased. This is seen in animals as increased sensitivity in paw pressure test and tail flick test, respectively. Clinical observations in heart patients have indicated that an elevated level of sympathetic tone was associated with an increased tenderness in specific acupuncture points, while other points on the body surface were unaffected. Similarly, a lower level of sympathetic tone was associated with a decrease in tenderness of these acupuncture points.

In an unpublished pilot study in 250 healthy people and conducted by the present inventors, a significant correlation was found between self-palpated degree of tenderness in specific acupuncture points when compared to non-acupuncture points and self-reported degree of stress. This observation has not previously been described as part of the warning system in animals and/or humans. From a teleological and evolutionary point of view, both aspects of stress modulation of nociception improve survival potential by using a mechanism, which can be demonstrated as far back in the evolution as the fish.

The present inventors have therefore realized that in case of an aversive environment the sensitivity of the polymodal receptors within the segmental innervation of the heart is increased simultaneously with the increase in the sympathetic activity. This warning system sensitivity (WSS) can be measured by means of the the apparatus and system described herein.

Serum Cortisol as a Measure for Transient Stress

Several studies indicate that serum or saliva cortisol may be useful indicators of transient stress. In patients presenting cardiopulmonary arrest to an urban emergency department, serum cortisol levels increased significantly during the first 6 hours after return of spontaneous circulation and decreased significantly during the following 18 hours. In people undergoing Coronary artery by-pass grafting (CABG), serum cortisol was found to be elevated after surgery. Measurements of serum Cortisol in 24-hours has been found to be useful in understanding the response to apoplexia, with higher levels of serum cortisol being associated with greater severity of the neurological deficit, larger ischemic lesions on CT and worse prognosis (p<0.05).

In people with short or long term unemployment, a decrease in physical performance was associated with an increase in serum cortisol and increased emotional disturbance, the latter evaluated from a questionnaire. In soldiers a positive correlation between serum cortisol and acute stress was found. In fish, an increase in serum cortisol has been found during expose to acute stress. However, this response was absent in fish exposed to persistent stress.

In women with early breast cancer, a cognitive-behavioral stress management program reduced serum cortisol, when compared to an un-treated control group. In an uncontrolled pilot study in women with primary breast cancer and 6 months observation period, a comprehensive stress-management program was found to lower middle blood pressure, decrease the number of clinical signs of persistent stress, self-evaluated stress level and decrease in degree of tenderness on specific locations on the chest. However, neither glycated hemoglobin nor serum cortisol was changed.

Pressure-Rate-Product (PRP) as a Measure for Transient Stress

In cardiology the Pressure-Rate-Product (PRP) serves as a practical index for myocardial oxygen uptake and thus reflects cardiac work. It is mainly controlled by beta-adrenergic catecholamines and as such it is a suitable marker for transient changes in sympathetic tone of the heart.

Glycated Hemoglobin as a Measure for Persistent Stress

Several studies indicate that glycated hemoglobin HbA1c may be used as a measure for persistent stress. In medical students HbA1c has been found to be elevated in students prior to an exam, when compared to other students. Furthermore, 4 months later HbA1c was significantly decreased in the exam group. These results are later confirmed in other studies. Among native Australians, when compared to Western Australians, an elevated HbA1c was found as a possible sign of the persistent stress of “westernization” among the native people. In patients with myocardial infarction and no diabetes, Hb1Ac and serum cortisol at time for admission correlated significantly to the 5.5 year survival. Persistent job strain has been found to be associated with elevated levels of Hb1Ac in both Japanese and Danish workers. Among burned out women a higher level of HbA1c was found, when compared to a control group. In a review on physical measures for stress, Kelly and Hertzman strongly recommends HbA1c as useful for measurement of persistent stress.

Blood Pressure as a Measure for Persistent Stress

Persistent stress is a strong pathogenetic factor in Metabolic Syndrome. Metabolic syndrome, affecting approximately 40 million Americans is associated with obesity, insulin resistance, hypertension and dyslipidermia, all of which are risk factors for cardiovascular disease and premature death. The increase in blood pressure with increasing age has been found to be related to persistent stress.

Systolic blood pressure changes more rapidly than diastolic blood pressure during transient stress. In an uncontrolled pilot study in women with primary breast cancer and 6 months observation period, a comprehensive stress-management program was found to lower middle blood pressure, decrease the number of clinical signs of persistent stress, self-evaluated stress level as well as Warning System Sensitivity. However, neither glycated hemoglobin nor serum cortisol was changed significantly.

Impaired Stress Response with Persistent Stress

An impaired cortisol stress response has been observed in fish exposed to environmental pollution. In rats, maternal deprivation has been found to produce persistent abnormalities in behavioral and neuroendocrine functions of the hippocampus, an important region of the brain in respect to the stress response. Maternal stress in humans, measured as reduced birth or infant weight, has been found to influence the stress response later in life, and with a marked gender difference. Two studies have demonstrated a blunted cardiovascular response to acute stress in otherwise healthy people exposed to persistent stress.

In a unpublished pilot study on opera singers, the present inventors have observed that when persistent stress is low, measured as low or normal HbA1c levels, a correlation is found between change in WSS and PRP from before to after performance, and this correlation could not be found, when the degree of persistent stress is high (measured as a high HbA1c level).

The present investors have found that persistent stress is asssociated with reduced elasticity during stress related to a performance or a mental stress test (see FIG. 28-30)

Choice of Treatment to Reduce Warning System Sensitivity (WSS)/Defense Reaction/Reflex sensitivity (DRS)

From a clinical perspective, the ability to measure WSS/DRS as a measure of stress is only interesting if the it can be positively influenced by a conscious effort. An unpublished clinical pilot study on 183 consecutive patients in a Danish and a Japanese acupuncture clinic, indicated that after 3 minutes of acupuncture, 94% of the patients experienced a decrease in WSS with a 35% decrease as the median effect. It has not been possible to identify interventions of other kinds with a similar response and effect rate. Accordingly, as the aim of the present example is to verify that an elevated Warning System Sensitivity measured by the method and apparatus of the present invention can be diminished, the results of the pilot study suggested the use of acupuncture treatment as a first choice. In other earlier published studies, acupuncture has been found to increase Pressure-Rate-Product (PRP) in heart patients and modulate PRP in healthy persons.

Design of Investigational Study 1

The primary aim of study 1 is to verify the inventive finding that when the level of transient stress is the same, persons with high serum levels of glycated hemoglobin (HbA1c) exhibit an elevated Warning System Sensitivity (WSS), measured by the method of the invention as an increased degree of tenderness in specific points of the skin, when compared to persons with low HbA1c.

The secondary aim of the study is to explore the following hypotheses:
a. Persons with a high Middle Blood Pressure (MBP) have a high WSS, when compared to persons with a low MBP.
b. Persons with a high PRP have a high WSS, when compared to persons with low PRP.

The trial will be an open, prospective, observational study.

Recruitment is made from private companies and by invitation in the media. 300 persons in total with will be included: persons with an expected high level of stress as well as persons with an expected low level of stress. Men and Women age 18-75 years old are included.

Exclusion Criteria: Diabetes Mellitus (insulin dependent and non-insulin dependent), Psychiatric disease, Pregnant women and women who is breastfeeding, Systemic steroid medication within the last 6 months, Previous measurement of WSS.

The selection of subjects is based on the fac that the included groups reflect the general healthy adult population.

The following procedure will be used:
Verbal and written information provided to the subject prior to the visit through a telephone call and a letter/email.
Signature of informed consent
Baseline information

The questionnaire is filled out before baseline measurements are conducted in order to exclude screening failures. All data are collected in printed form. All data will be transferred to a SPSS database prior to analysis. A screening and enrolment log will be used.

The study will be conducted on the location of the company or at the inventors' acupuncture centre.

The individual effect variables are measured as follows:

Questionnaires
Demographic data; age, sex, BMI, waistline, concomitant disease, concomitant medication, own perception of stress level

Physiological Measures
Glycated hemoglobin (HbA1c) will be measured from capillary blood; i.e a blood sample from the fingertip of the subject. It is used as a measure for persistent stress.
Blood pressure and pulse rate will be recorded by automatic monitors.
Middle blood pressure (MBP) is calculated as the median of 3 automatic measurements within one minute—and is used to reflect the general level of long-term sympathetic tone of the cardiovascular system as is used as a measure for persistent stress (see introduction).
Systolic blood-pressure-heart-rate-product (PRP) is calculated as an indicator of cardiac work and myocardial oxygen consumption, thus reflecting the transient sympathetic tone of the heart (see introduction).
Warning System Sensitivity (WSS) measured as degree of tenderness is measured on specific acupuncture points on the chest (Nussbaum & Downes 1998). It is measured by StressZensor© and used as a possible measure for sympathetic tone, according to patent (Ballegaard 2004). WSS is measured on the left index finger (on dorsal site of middle phalanx) and on acupuncture point CV 17 (see introduction). Presence of “withdrawal reflex” in association with the measurement will be registered.

Primary Effect Variables

WSS in respect to HbA1c.

Secondary Effect Variables
WSS in respect to MBP.
WSS in respect to PRP.

The subjects are asked to have no food, tea, coffee, smoking, alcohol within the last 2 hours prior to the examination.

All persons with a WSS level >40, will be invited to participate in the randomised trial described below (“investigational study 2”), in which short term acupuncture is compared to rest placebo with respect to lowering the WSS.

The subject may withdraw at any time.

Blinding

Subjects are blinded by the hypothesis of the study: the level of WSS being determined by the level of glycated hemoglobin—and this information being unknown to him or her. Concerning researcher blinding, the researcher conducting the WSS measurement cannot be blinded. Accordingly, two teams of researchers work together, each being blinded to the result of the other team. One team measures WSS and the other HbA1c, blood pressure and pulse rate. The use of automatic machinery for measurement of blood pressure, pulse rate, and HbA1c helps blinding the researcher, who conducts these measurements. Furthermore, the subject is told not to have verbal communication with the observers, except when during SO measurement, to say stop when pressure-pain-threshold has been reached.

Sample Size Calculation

From previous studies (Breast Cancer & Opera) it is expected that the mean WSS will increase 10 units for each increase of 1 in HbA1C. It is also observed that male subjects have considerably lower values of WSS. Based on experience from these studies it is assumed that the different levels for male/female can explain 43% of the total variance, and that the combination of sex and HbA1c can explain 45% of the total variance. Testing the hypothesis of no influence of HbA1c using a F-test with α=0.05 (Type I) the following powers can be achieved:

| | | | | | |
|---|---|---|---|---|---|
| N = | 200 | 250 | 300 | 350 | 400 |
| β = | 77% | 85% | 91% | 95% | 97% |
| (Type II) | | | | | |

Thus the planned sample size of 300 results in an acceptable power of 91%;

Statistical Methods

The primary analysis will be made using linear regression of WSS on HbA1c controlling for sex and age. The primary hypothesis will be tested using a type III F-test for the inclusion of HbA1c in the model.

Subsequently, an explorative analysis will be made including any linear regression of WSS on PRP and MBP on above mentioned model.

Design of Investigational Study 2

This study described in the following aims to verify the following hypotheses:

Primary aim: When compared to a placebo pill and rest, acupuncture decreases Warning System Sensitivity

Secondary aims are to verify the findings of the above-mentioned pilot studies that:
a. Persons with high level of HbA1c have a decreased elasticity of their nervous system, measured as a blunted response with respect to Pressure-Rate-Product (PRP), MBP, saliva cortisol and Warning System Sensitivity, when exposed to a standardised acupuncture treatment.
b. Persons with a low level of persistent stress, measured as a low level of serum HbA1c, have an elastic nervous system, measured as a significant and concomitant response with respect to PRP and WSS, when exposed to standardised acupuncture treatment.

A prospective, single blinded, between-group comparison study, including three treatment arms will be performed. The study involves 100 consecutive persons, who meet the inclusion criteria, randomised to one of three treatments: acupuncture, placebo and rest.

Inclusion criteria: Men and Women age 18-75 years old, with a WSS>40.

Exclusion criteria: Diabetes Mellitus (insulin dependent and non-insulin dependent), Psychiatric disease, Pregnant women, and women who is breastfeeding, Systemic steroid medication within the last 6 months, Previous measurement of WSS, Acupuncture treatment within the last 6 months.

The selection of subjects for the study is based on the following:
1) The hypotheses of the study were generated through a pilot study based on consecutive adult patients at an acupuncture clinic.
2) Persons included represents the general population, have an elevated Warning System Sensitivity, have not previously been measured by use of the apparatus and method of the invention and are not regular users of acupuncture.

In the randomised clinical trail the following procedure will be used:
1) Verbal and written information provided to the patient prior to the visit through a telephone call and a letter/email.
2) Signature of informed consent
3) Baseline information
4) Randomisation to either one of three possible treatments (acupuncture, placebo pill or rest).
5) 3 minutes run-in period with the patient in supine position
6) Pre-treatment measurements: saliva cortisol, pulse rate, blood pressure, Warning System Sensitivity measured by the method of the present invention.
7) 3 minutes of treatment with the patient in unchanged position: Either acupuncture treatment, placebo or rest.
8) Post treatment measurements: saliva cortisol, pulse rate, blood pressure, Warning System Sensitivity measured by the method of the invention.

The placebo is in form of a vitamin tablet, which dissolves after sublingual administration. For acupuncture treatment, points Zuzanli (St 36) are used bilaterally. The needles (disposable stainless steel needles, Serin©) are inserted perpendicular to the skin into the underlying muscle, to a depth of approximately 5 mm with no further mechanical or electrical stimulation and left in situ for 3 minutes. Rest is in the study defined as staying in the supine position with no further action taken place, either by experimenter or by subject.

For the randomised trial, all measurements and treatments will be conducted at the inventors' acupuncture centre.

The individual effect variables are measured as follows

Questionnaires
Demographic data; age, sex, BMI, waistline, concomitant disease, concomitant medication, own perception of stress level, expectations concerning the effect of the three allocated treatments.
General health.
Presence of clinical sign of persistent stress.
Presence of occupational stress (National Institute of Occupational Health) To AMH. International references for validity of the questionnaire

Physiological Measures
Glycated hemoglobin (HbA1c) will be measured from capillary blood; i.e a blood sample from the fingertip of the subject. It is used as a measure for persistent stress.
Blood pressure and pulse rate will be recorded by automatic monitors.
Middle blood pressure (MBP) is calculated as ⅔×diastolic blood pressure+⅓×systolic blood pressure. The median of 3 automatic measurements within one minute is used. MHP is used to reflect the general level of long-term sympathetic tone of the cardiovascular system and used as a measure for persistent stress (see introduction).
Saliva cortisol will be collected by a saliva sample on location, while analysis of the saliva will be conducted in a special laboratory. It is used as a measure for transient stress.
Pressure-Rate-Product (PRP) is calculated as an indicator of cardiac work and myocardial oxygen consumption, thus reflecting the transient sympathetic tone of the heart.
Warning system sensitivity (WSS) measured as degree of tenderness is measured on specific acupuncture points on the chest described herein. It is measured by the method of the invention and used as a measure for sympathetic tone. WSS is measured first on the left index finger (on dorsal site of middle phalanx) and subsequently on acupuncture point CV 17.

Primary Effect Variables

In between-group differences of WSS in respect to the three treatment arms; acupuncture, rest and a placebo pill.

Secondary Effect Variables
In between-group differences in respect to the three treatment arms concerning PRP, MBP and saliva cortisol.
Correlation between changes in WSS and changes in PRP, MBP, saliva cortisol with respect to pre-treatment level of glycated hemoglobin (HbA1c).

Expectations concerning the effect of the three allocated treatments will be obtained at baseline.

As a true blinding in acupuncture trials is not possible, due to the very nature of the treatment, a special design is used, combining psychological, physiological and clinical effect variables.

Patient expectations concerning each of the treatments are measured before the start of the treatment. The use of a placebo, the unknown level of serum HbA1c as well as the use of objective physiological variables serve to blind the patient. The treating researcher cannot be blinded for obvious reasons. However, the influence of the treating researcher's expectations are eliminated by the hypothesis of the study: the effect of the treatment being determined by the pre-treatment physiological state of the subject—and this information being unavailable to him. The observing researcher will conduct all measurements and will be blinded towards the choice of treatment, as the acupuncture, placebo treatment and rest having the same appearance from the observers location (i.e. the legs of the patients (on which acupuncture point ST 36 is located) will be covered by a piece of cloth during all treatments. The observing researcher will be out of the treatment room at the time of randomisation and when the allocated treatment is conducted. Furthermore, by the use of automatic machinery, observer expectation can be eliminated. The subject is told not to have verbal communication with the observer or the acupuncturist, except when during WSS measurement, to say stop when pressure-pain-threshold has been reached. Between the observing researcher and the acupuncturist, communication is concerning the timing of the procedure, only.

Information of Patients

The patients receiving the placebo are informed that they receive a tablet, which is believed to have the same effect as acupuncture. However, the exact effect of the pill cannot be disclosed, as this would induce an expectation effect in the patient. The patients are informed that the aim of the study is to record the physiological changes, which may occur during the two kinds of treatment, and compare these changes to the changes, which may happen during rest.

The patients are asked to take in no food, tea, coffee, and alcohol and not to smoke within the last 2 hours prior to the examination.

Randomisation Procedure

The randomisation procedure is based on a computer program. Choice of randomisation will be known to the treating researcher and patient, only. Choice of randomisation is unknown to the statistician until the database is released for statistical analysis.

The subjects may withdraw at will at any time. Adverse event during the trail will be registered. No major risks are expected.

The study will be submitted to “Datatilsynet” in Denmark in amble time prior to the initiating of the study, and will not be initiated until approved by “Datatilsynet”.

The primary analysis will be based on all randomised subjects according to the intention-to-treat principle (ITT).

Statistical Considerations

Sample Size Calculation

Based on previous observations WSS is expected to decrease 35% after 3 minutes of acupuncture, and the standard deviation (SD) of the relative change of WSS is expected to be 25%. Testing the hypothesis of no difference between acupuncture and placebo treatment using a t-test with α=0.05 (Type I) will result in the following powers (Type II error) when the total sample size is 100 subjects (33 in each treatment arm):

| | | | | | |
|---|---|---|---|---|---|
| Relative change of WSS | 20% | 18% | 16% | 14% | 12% |
| In Placebo group | | | | | |
| β = | 67% | 78% | 86% | 92% | 96% |
| (Type II) | | | | | |

Thus the planned sample size of 100 results in an acceptable power around 90%, if WSS decreases 15% in the placebo treatment group.

Statistical Methods

The primary analysis will be based on the relative change of WSS calculated as:

WSS_{after treatment}−WSS_{before treatment}/WSS_{before treatment}

The test of the hypothesis of no difference between acupuncture and placebo treatment will be done using t-test with α=0.05.

The study will be approved by the local Ethics Committee.

The above described findings in pilot trials have opened for novel aspects of the invention:

First of all, a method for determining the status of warning system sensitivity (WSS) in a subject is provided, comprising determining an applied stimulation at a threshold value in point(s) on or in the body where nociception is dependent on sympathetic tone and correlating the stimulation threshold with a WSS value. Hence, the herein described determinations of stress and sympathetic tone can be supplemented with measurements of WSS, given that the right set of circumstances are present. For example, measurements of WSS are optimized when the subject is in an environment substantially free from acute stress-inducing factors or where it can be established that no acute stress-inducing factors seem to be present. Likewise, the environment (or the person's activity level) should preferably be free from factors which would in their own right induce an increase in sympathetic tone (for instance, the subject should preferably not be in a state following shortly after strenuous heavy exercise because this will increase the sympathetic tone). In such a situation, the WSS readout of the method of the invention will indicate to the skilled artisan whether the person has an increased nociception and hence an increased WSS—this may in turn indicate the need for some kind of clinical intervention.

In general, all teachings herein relating to the technicalities involved in measurements of sympathetic tone and stress (choice of types of stimulation etc) apply mutatis mutandis to the method for determination of WSS.

As mentioned above, the findings relating to WSS also opens for possible intervention so the findings also open for a method for modulating the status of warning system sensitivity, the method comprising a) determination of sympathetic tone and/or the stress level and/or WSS in a patient described herein and if the determination indicates an elevated sympathetic tone and/or level of stress, subjecting a sympathetic tone dependent point to a stimulation having a lower intensity than the stimulation threshold value for a period of time. This subjection of a lower intensity is preferably performed by means of acupressure or acupuncture therapy.

### Example 12

### Pressure-Pain-Threshold Measured by Algometry

Warning and defense systems have been an essential part of the survival strategy of living organisms throughout the entire evolution. On the skin, special sensors have developed in order to detect potentially tissue-damaging environmental stimuli, providing the organism with the information needed for an optimal response to adverse environmental conditions—and it may be accompanied by a reflex response such as startle or withdrawal.

This nociceptive system is based on a polymodal receptor, an undifferentiated nerve cell of identical nature throughout the evolutionary chain, from fish to higher vertebrates and humans. The receptor is stimulated by mechanical pressure (noxious as well as non-noxious), temperature and acidity: Two distinct classes have been identified; A-delta receptor with a low pain threshold and C-receptor with a high threshold, both susceptible to modulation (1) though the Cation channel subgroup TRPV 4 (TRPV=Transient receptor potential vanilloid). A variety of exogenous and endogenous substances, including sympathetic as well as parasympathetic input, Khasar S G 2003), may cause modulation, thus creating gating diversity.

Psychological stress is known to suppress the pain sensation of these sensors (stress-induced analgesia), providing survival potential by suppressing general pain sensation in fight or flight or just as a salivating gesture from nature in the final defeat. The opposite, with an increase of pain sensation (stress-induced hyperalgesia) has also been observed, mainly locally in relation to trauma or injury or as an enhanced tale-flick or paw-pressure response in animal during restraint stress, each providing survival potential by enhancing the sensitivity of the warning system. Similarly, visionary input causing psychological stress has been found to enhance hearing, and olfactory input to augment the startle reflex.

The existence of points on the skin with increased sensitivity, and specifically related diseased organs/regions have been a cornerstone of the Oriental acupuncture tradition for centuries, and clinical observations in ischemic heart disease patients have indicated that an increased tenderness in specific acupuncture points on the chest bone may be associated with increase in sympathetic tone and stress, while other points on the body surface were unaffected. The link between stress and ischemic heart disease is strong. Furthermore, a multifarious treatment complex, in which patients used the degree of tenderness as a biological feedback marker for the stress, gave the patients a survival rate superior to that of the general population.

On this background, we hypothesized that psychological stress increases the sensitivity of the chest bone acupuncture point CV 17, located in level with the nipple and the forth intercostals space.

We first measured the pain threshold (PT), using algometry in 48 members of the Royal Opera Chorus before and after an opera performance. With 5 seconds in-between measurements, a significant correlation between first and second measurement was found, (r=0.92; p<0.0001). PT was significant lower after performance, when compared to before performance (mean 33.6 versus 38.9) (p<0.05).

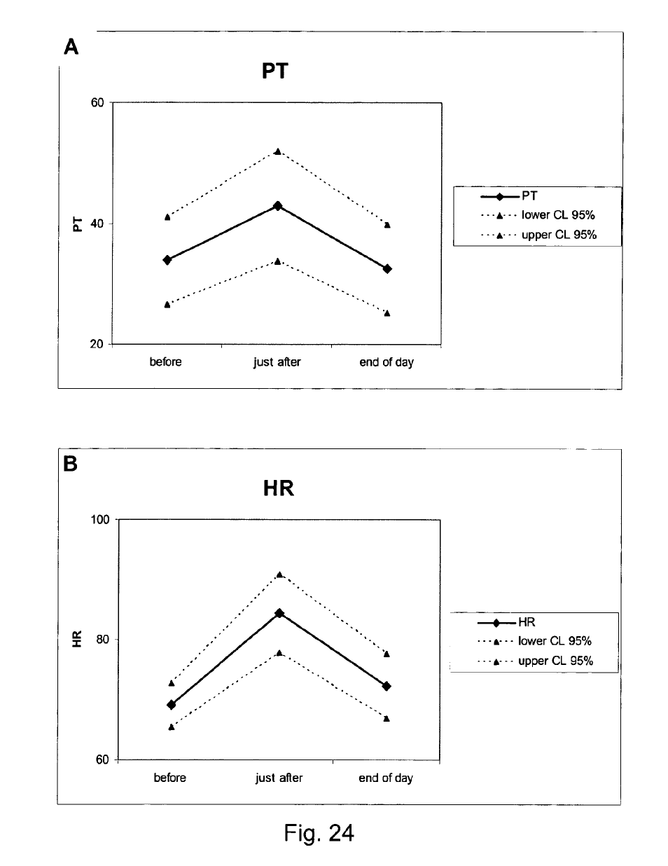

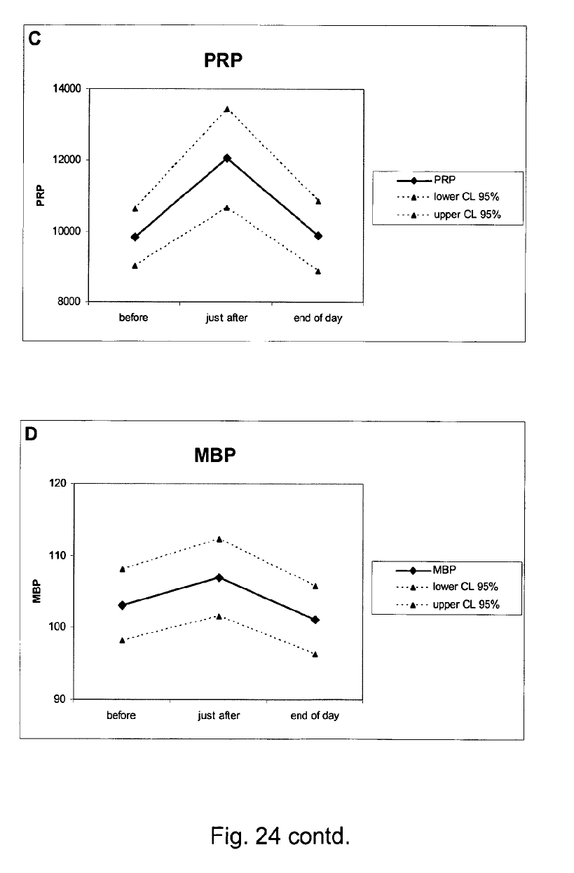

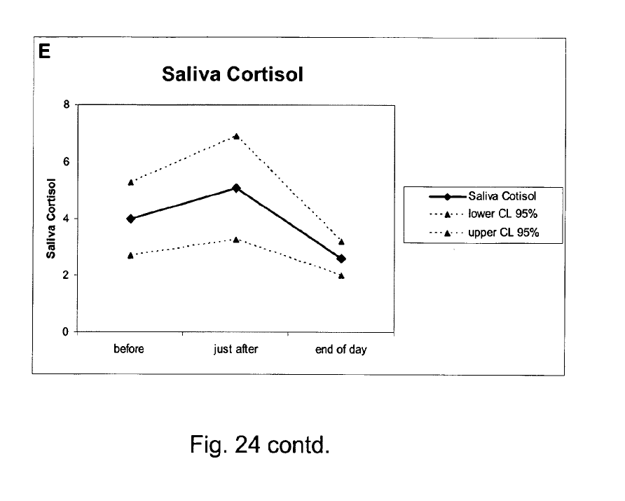

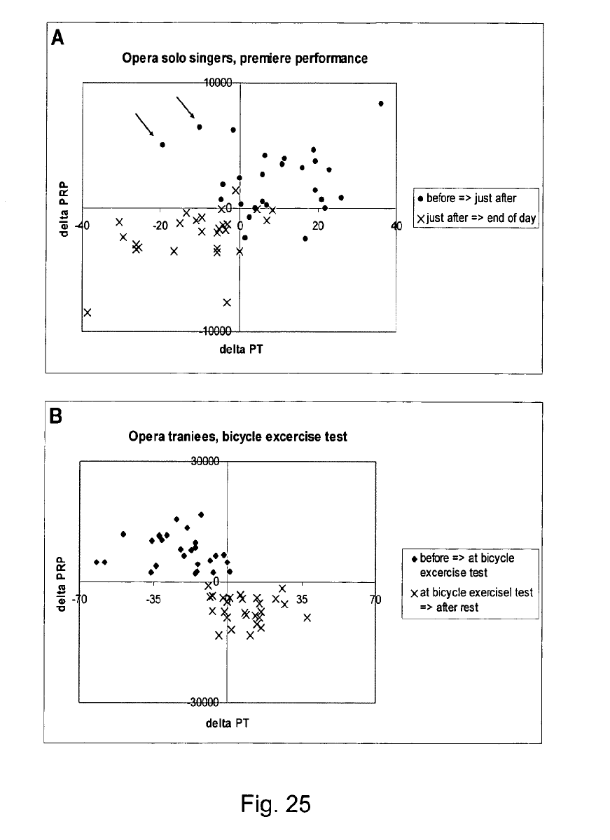

Secondly, to increase fluctuations in the recorded psychological stress levels, solo singers, preferable during an opening-night performance, were measured: 1) before the start of the performance; 2) right after the top of their performance and 3) at the end of evening. For effect variables the Pressure-Rate-Product(PRP), the Heart Rate (HR), the Middle Blod Pressure (MBP) and saliva cortisol were included. FIG. 24 shows that changes were significant for all effect variables. Changes in PT correlated significantly to changes in PRP (r=0.54, p<0.0001) (see FIG. 25a), HR (r=0.55, p<0.0001), MBP (r=0.42, p<0.005), and salivary cortisol (r=0.28, p<0.05).

With respect to the correlation between changes in PRP and PT, two measurements deviated from the general pattern, as indicated by arrows in FIG. 25: an increase in PRP being associated with a decrease in PT. An exploration of possible explanations revealed that compared to the rest of the singers, these two singers have had a high level of physical activity in the last minutes before the second measurement (running across the stage and climbing a ladder).

Subsequently, 26 opera trainees underwent a bicycle exercise test, simulating the exercise patterns of the opera singers on stage. FIG. 25b, shows the negative correlation between changes in PRP and PT during the bicycle exercise (r=−0.70; p<0.0001). In addition, the trainees were measured in relationship to a singing audition, with the finding that changes in PRP and PT correlated significantly (r=0.32; p<0.01).

These studies suggest that when healthy people are exposed to a psychologically stressful situation, this is associated with an increased sensitivity of chest bone acupuncture point CV 17, and when the situation resolves, the sensitivity is reversed. The initial observations in opera chorus singers were confirmed under improved experimental conditions, first in opera solo singers and later in opera trainees. Changes in sensitivity correlated to known physiological and biochemical stress variables. In addition, the inclusion a physical exercise study revealed that, although the physiological changes of the cardiovascular system during an opera performance are similar to those of physical exercise, they relate to different phenomena: psychological stress and physical exercise, with the opera performance containing a combination of the two.

Exercise is known to reduce pain sensitivity and to reduce psychological stress. On this background it is concluded that changes in pain sensitivity of the chest bone acupuncture point reflect different levels of psychological stress.

One aspect of measurement of psychological stress is the ability to record transient changes in stress. A second aspect is the ability to measure baseline levels. Accordingly, resting values of pain threshold of acupuncture point CV17 and the index finger were recorded together with HR, PRP and MPT in 181 consecutive patients in an outpatient medical acupuncture clinic. Resting PT on CV 17 correlated significantly to baseline PRP (r=0.23; p<0.01) and HR (r=0.25; p<0.01). A significant correlation to MBP was present among the 63 women with elevated PT, i.e. PT>45 (r=0.30; p>0.05). Females had a significantly higher PT (mean 46), when compared to males (mean 32) (p<0.0001). The median PT on the index finger was 0, compared to 39 on acupuncture point CV17 (p<0.0001), and accordingly measurement of the PT on the index finger did not exhibit a useful sensitivity.

HR, PPR and MBP have all been found to be useful variables in stress related conditions and prognostic indicators in cardiovascular disease. Furthermore, in apparently healthy men, resting heart rate has found to be associated with a micro-inflammatory response. The common denominator has been suggested to be sympathetic activation. Thus, the finding of a significant correlation between sensitivity of the chest bone and cardiovascular variables, supports the hypothesis of the study.

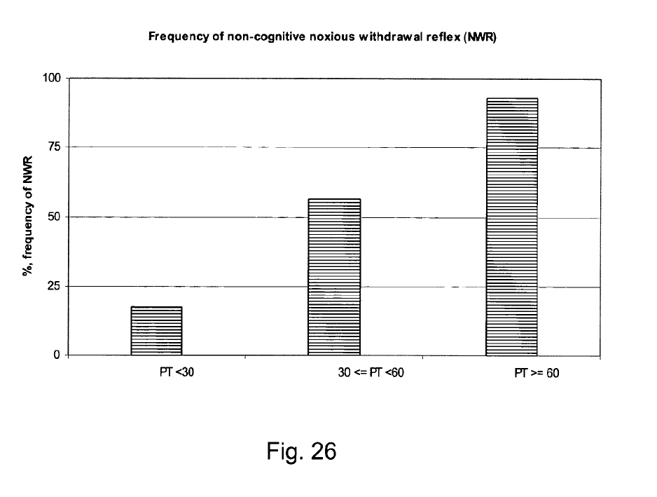

FIG. 26 shows the connection between the PT and frequency of noxious withdrawal reflex NWR among patients divided in three groups, with a 100% increase in pain sensitivity separating each group. Among patients with low sensitivity (PT<30), a withdrawal reflex was observed in 17% of the patients, compared to 56% in the middle group, and 93% among patients with highest sensitivity (PT>60) (p for group difference<0.0001; correlation coefficient between PT and presence of NWR; r=1.0).

The withdrawal reflex has been a survival device throughout the evolution—as a defensive response to a potential damaging stimulus and represents the simplest centrally organised response to painful stimuli. This reaction helps the animal to move away from the noxious stimulus. It is likely that all nervous regulatory systems have been built upon the foundation of this primitive withdrawal reflex. A variety of NWR exists in both animals and humans, with each NWR being regarded as a poly-synaptic functional module, depending on a specific area of stimulation. In horses, a dose-dependent relationship between stimulus dose and NWR magnitude has been observed, and observer concordance was high. Differentiation of the withdrawal reflexes improves survival potential: Injurious stimuli augment the protective function of reflexes by enhancing (sensitising) reflexes that protect the injured site and inhibiting those reflexes that might exacerbate the insult.

Accordingly, the hypothesis of the study, namely that the warning system may increase sensitivity of vital areas in order to enhance survival, is supported by the present findings. The survival potential is improved through enhancement of the simplest non-cognitive efferent motor response: the withdrawal reflex. On a more sophisticated response level, the survival potential may be improved for the individual, who through repetitive self-measurements and cognitive processing of the achieved information succeeds in preventing suboptimal behavioral patterns associated with prolonged elevated levels of stress hormones.

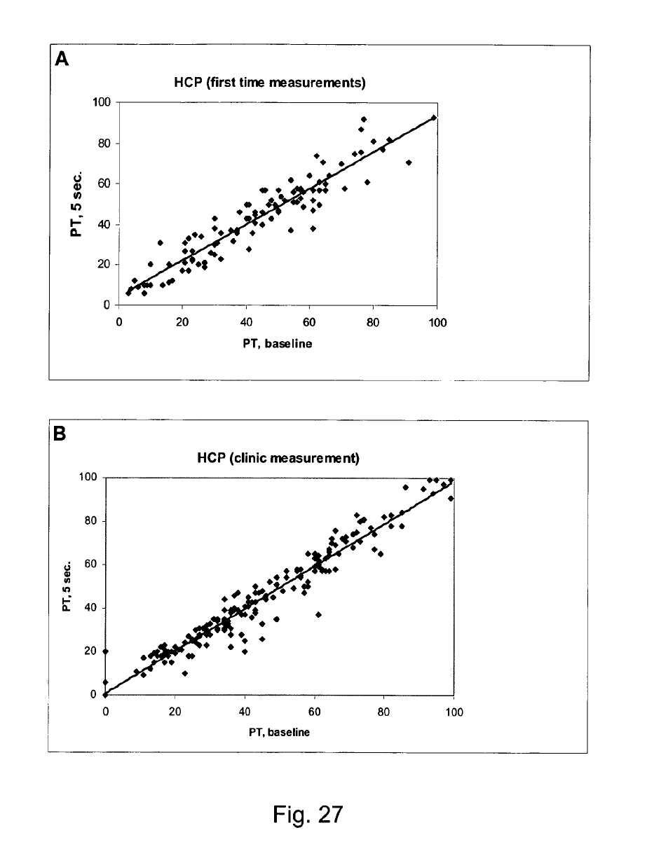

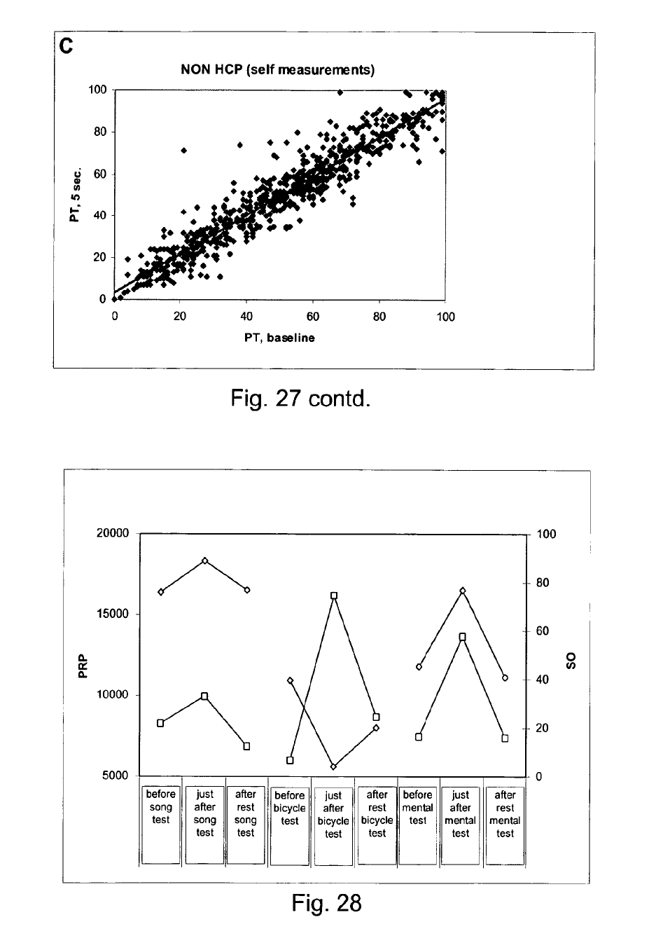

FIG. 27 shows the results of the reliability testing: 27a) Measurement conducted by HCP in 82 healthy people, who are measured for the first time (r=0.94, p<0.0001), 27b) measurement conducted by an HCP in 181 consecutive patients in a medical outpatient clinic (r=0.97; p<0.0001) and 27c) measurement conducted by 33 NON-HCP's as self measurement, conducted twice daily during a two week period (r=0.95; p<0.0001).

The observed level of reliability of PT measurement matches the level of contemporary non-chemical non-invasive diagnostic methods: repeated audiometric measurement (r=0.70), armpit versus rectal temperature (r=0.73) and rectal versus core temperature (r=0.94).

Pain is an active process generated partly in the periphery and partly within the central nervous system by multiple changes that together determine the gain of the system,with the overall aim to create optimal survival potential (Woolf C J & Salter M W 2000). In line with this, pain sensation is influenced by cognitive and emotional processing in the brain, attention as well as social factors.

Physiological pain is initiated by special sensory receptor fibers innervating peripheral tissues: the polymodal receptor. The sensory input activates neurons in the spinal cord, which project to the brain cortex via relay stations in the thalamus. A noxious input also activates a withdrawal reflex, as well as emotional, autonomic and neurohumoral responses.

Modulations of the sensitivity are reversible changes in the excitability of primary and central neurons mediated by alterations in receptors/ion channels by intracellular signal transduction cascades. An increase in pain sensitivity may lead to a situation in which stimuli that would never normally produce pain, do so (allodynia) or noxious stimuli produce more pain that usual (hyperalgesia). Such sensitisation may take place peripherally as for example in inflammation, or centrally as in repeated C-fiber stimulation. Depression of spinal inhibitory mechanisms may contribute to the centrally mediated sensitisation. Activation by the neurotransmitter gamma-aminobutyric acid (GABA) of A-delta afferent fibers accounts for this depression. Selectively activating of GABA(B)-receptor-bearing RAIC neurons produces hyperalgesia through projections to the Amygdala, an area involved fear and thus psychological stress.

Transient receptor potential (TRP) channels represent a family of Cation channels, which are expressed in almost all cell types in both invertebrates and vertebrates. They are expressed in nearly every tissue and cell type. On the polymodal receptor cell one subfamily of the TRP channels; the TRPV 1-6 family is involved in the perception of temperature, pain and acidity. The Subgroup TRPV 4 is specially related to pressure with the sensation being transmitted through A as well as C fibers, and with pronounced modulatory diversity, reflecting sympathetic as well as parasympathetic input.

On this background, it may be concluded that a functional, structural and molecular basis for the observed modulation is present, with the A-delta polymodal receptor being the mediator, and the TRVP 4 Cation channel being the molecular site of the modulation.

The present series of studies in this example shows that PT measurement on the chest bone acupuncture point CV 17 represents a new method to measure transient changes as well as baseline psychological stress. At present, no international consensus exits concerning measurement of stress in general. As the method is reliable and easy to use for both professionals and non professionals, the present findings suggest a broad range of practical applications.

Psychological stress is a strong pathogenetic factor in metabolic syndrome, affecting approximately 40 million Americans, and is a risk factor for cardiovascular disease and premature death. Apart from this obvious application in the risk- and prognostic management of cardiovascular disease, the healthy individuals of the population may benefit from the method, in terms of recording of the psychological implication of their working environment as well as their general life and life style, providing the potential for appropriate cognitive reflexions and actions. In animal farming, future research may focus on the potential of the method to help ensuring a non-stressful living environment as well, with significant impact on the nutritional quality of the associated products.

Specific Method Steps:

An algometer with the following special features was used:
1) Algometer readings were hidden until the measurement was completed in order to blind the subject and researcher. For analysis, the mean of two consecutive measurements was used.
2) The applied algometer pressure was mathematically transformed into a logarithmic scale of sensitivity levels similar to the Decíbel scale. Algometric Measure (PT)=Log 200−Log 100 (100*threshold in kilo/14 kilo)). An increase in 30 PT units corresponds to a 100% increase in sensitivity (=lower pain threshold).
3) In order to prevent damage on the skin, an alarm sound was activated when pressure reached 14 kilograms.
4) A special rubber measurement foot plate was incorporated with the aim to allow determination of pressure pain threshold on the bone, without applying noxious stimulation to the skin, as such a stimulation may lead to determination of skin pressure threshold instead.

Conduction of pain threshold measurement: All measurements were carried out with the subject in the supine position. The subject was instructed to say: “stop” as soon as discomfort or pain was felt. If the researcher observed a startle or withdrawal reflex, the measurement was stopped. Each measurement session started with 2 measurements at the index finger, and subsequently two measurements on the chest. In the study of opera trainees (cf. below), the first measurement of the day consisted of two measurements. For the remaining measurements, only one measurement was used in order to prevent damage of the sensory receptor.

Measurement locations: 1) Active point: Acupuncture point CV 17 on the middle of the chest bone, identified by palpation by the observer as the most tender point within a circle of 5 centimeter in diameter from the Chinese description of the point in level with the nipple and the forth intercostal space. 2) Control point: on the dorsal part of the middle phalanx on the left index finger.

Blood pressure and heart rate were recorded by a Thuasne automatic blood pressure monitor, model W0840 002 001 (Microlife ref. BP-3AA1-2, BP 243-92307 Levallois-Perret Cedex, France). For analysis, the mean of two consecutive measurements was used. Pressure-Rate-Product (PRP) was calculated as systolic blood pressure×heart rate (HR). Middle blood pressure (MBP) is calculated as ⅔ diastolic blood pressure×⅓ systolic blood pressure. Saliva cortisol samples were obtained on location and analysed by a radio-immunoassay method.

Blood pressure, heart rate, pressure-rate-product and saliva cortisol are expected to change when the level of psychological stress changes (R. E. Noble, Metabolism 51(5), 37-39 (2002); B. Hari, B. Wesisshuhn, H. H. Kerscbaum. Neuro Endocrinolog Letter 27(5), 669-674 (2006).

Noxious withdrawal reflex (NWR) during measurement was recorded as the presence of in-voluntary muscle contractions in the regions of eyes, cheeks (=startle reflex) or in the flexor muscles of the neck and upper extremity. NWR is regarded as a reliable and objective tool for exploring pain control systems in humans (V. Skljarevski, N. M. Ramadan, Pain 96, 3-8 (2002)).

Subjects and Study Design

Opera singers in general were expected to be exposed to performance related psychological stress (D. T. Kenny, P. Davis, J. Oates, J Anxiety Disord. 18(6), 757-777 (2004)), and opera solo singers were expected to be exposed to an extraordinary high level of stress at the peak of their solo performance, especially at a premiere performance.

Study 1: Changes in Pain Threshold (PT) and Physiolocical Variables for Stress in Conditions with Predictable Changes in Stress

Phase A: 48 members of the Royal Opera Chorus were measured before and after a 3-hour opera performance. As the PT measurement was found to be reliable and was recorded as lower before than after the performance, the initial hypothesis was confirmed, and phase B was established.

Phase B: The experimental set-up was changed in order to increase the changes in recorded psychological stress and decrease the influence from confounding factors: Only solo singers were included (N=26; 16 women and 10 men), and preferably during a premiere performance. Their mean age was 46 years. Three measurements were recorded on the same day: 1) before the start of the performance; 2) just after the top of the performance at the expected peak level of their stress and 3) at the end of evening: i.e. as late as possible after the top performance, allowing maximum restitution within the limitations of the study logistics. The first and third measurements were conducted in the dressing room area of the singers, while the second measurement was conducted at a temporary backstage measurement station in order to minimize the time period between peak performance and measurement. For effect variables PRP, HR, MBP and saliva cortisol were included.

Phase C: When changes in PRP and PT were recorded for the opera solo singers, two measurements deviated from the general pattern (indicated by arrows in FIG. 25a): an increase in PRP was associated with a decrease in PT. An exploration of possible explanations revealed that compared to the rest of the singers, these two have had a high level of physical activity in the last minutes before the second measurement (running across the stage and climbing a ladder). Accordingly, phase C was established in which 26 opera trainees participated in two different tests on separate days: a singing audition and a bicycle exercise test. Each test was initiated and concluded with 10 minutes of rest in the supine position. Furthermore, the subjects were asked not to take any medication, coffee, alcohol or tobacco 2 hours prior to the tests and were asked not to endure in heavy physical activity during the last hour before the test. Their mean age was 27 years, 11 were males and 15 females.

The singing test consisted of two measurements: 1) as close as possible to the singing audition, but after 10 minutes of rest and 2) right after the audition, but again after 10 minutes of rest right. Accordingly, any recorded changes in PRP and PT were related to the restitution following the audition.

The bicycle test was conducted on a Kettler Ergometer bicycle, model X_{c} (X³) with Siemens elektronik cadiofitnes/SD 4, 8,5,9. Freizeit Marke Kettler, Heins Kettler GmbH & Co, KG post fach 1020, D-59463 Ense-Parsit, Germany, and consisted of 4 measurements: 1) after 10 minutes of rest, 2) after 2 minutes of exercise aiming at increasing PRP with 25%, when compared to resting PPR, 3) after another 2 minutes of exercise with the aim to increase PRP with a minimum of 40%, when compared to resting PRP, but below with a maximum workload of 80% of the age-estimated maximal workload/heart rate. The 4^{th} measurement was after 10 minutes of rest.

Depending on the estimate of the individual trainee concerning her or his physical fitness condition, the initial working load was 50/75 Watt and 80 revolutions per minutes (rpm) for women and 75/100 Watt and 80 rpm for men. For the second exercise phase, the workload was increased to 75/100/125 Watt and 90/100/110 rpm for women and 100/150/200 Watt and 90/100/110 rpm for men and with the possibility to increase bicycle time to 3 minutes. One subject was excluded as his PRP increased from 9700 to 33.300 during second exercise test with a heart rate of 197 (80% age-adjusted limit for heart rate=154).

Study 2 and 3: Resting Values of Pain Threshold (PT), Physiological Variables for Stress, and Presence of a Noxious Withdrawal Reflex (NWR)

181 consecutive patients (126 women and 55 men) at a private medical outpatient clinic were included. Their average age was 58 years. Their diagnoses were; cancer 55; heart disease 49, stress 19 and others 58. Resting values of PT, blood pressure, heart rate and the presence/absence of NWR were conducted in the supine position after 10 minutes of rest and blindly by two separate research teams; PT and NWR by the one team; blood pressure and heart rate by another team. For determination of PT, measurement on the acupuncture point CV 17 on the chest bone was regarded as the active measurement point, and measurement on the index finger as control measurement site. For clinical use, the PT scale has been made into a 3-step scale, for every 100% increase in sensitivity; PT<30; 30<=PT<60, PT=>60. The same is used when comparing the PT measurement and NWR.

Study 4. Reliability Testing of PT Measurement

Reliability testing of the PT measurement has been conducted in three situations, each of which the PT measurement were conducted twice with 5 seconds between measurements: 1) measurement conducted by HCP in 103 healthy people, who are measured for the first time, 2) measurement conducted by an HCP in 181 consecutive patients in a medical outpatient clinic and c) measurement conducted by 33 NON-HCP's as self measurement, conducted twice daily during a two week period.

Statistics

Statistics were carried out by an independent statistician, with the use of SAS software. 5% was used as significance limit.

Ethics

All subjects were given verbal and written information and signed a written content before first measurement. The local ethical committee approved the study in opera singers and trainees. The remaining study was conducted as part of the daily medical work at the clinic according to Danish medical regulations and ICH Guidelines for good clinical practice (7).

### Example 13

This example demonstrates 1) creation of peak performance during periods of transisent stress, 2) use of measurement of elasticity as a distinction between transient and persistent stress and 3) the effect of 12 weeks of self-measurement, cognitive processing of the achieved information and conduction of appropriate actions. Cf. FIGS. 28-30.

Physiolocical Background:

A prior condition for a peak performance at a situation with transient stress, is an activation of the stress response of the organism. This shows as an increase in WSS and DRS

To conduct the stress response, the brain needs to work extra, thus increasing the demand for oxygen. Similarly, for the muscles, which carry out the physical aspects of the defense response. Accordingly, the heart needs to work more. This shows as an increase in heart work measured as PRP (Heart Rate×Systolic Blood Pressure Product)(PRP), Heart Rate (HR), Middle Blood Pressure (MBP).

The above mentioned cardio-physiological variables are affected, not only by stress, but also by physical work—for example mild to moderate exercise on a bicycle. Consequently, these measures cannot be stand-alone measures for stress, but present usefull measures for stress, when combined with WSS and DRS.

Elasticity denotes the ability of the organism to appropriate adjustment of function in response to change in circumstances. Good elasticity means that the organism adjusts quickly and adequatly to such changes. In respect to a specific situation of transient stress, this means that the stress response is activated sufficiently at the peak of the performance and that homeostasis is reestablished quickly, when the situation is over.

In this example, three separate test situations are used:
a) Test at a singing audition: measurement conducted at the following periods: 1) after 10 minutes of rest and before the audition, 2) right after the audition, and 3) after 10 minutes of rest just after measurement no 2.
b) Test at a bicycle test 1) after 10 minutes of rest 2) after 4 minutes of exercise which in respect to increase in the work of the heart equals that of a singing audition, and 3) after 10 minutes rest.
c) Test at a 10 minute-mental stress test using arithmetics in combination with time and performance strain: 1) after 10 minutes of rest, 2) twice during the test and 3) after 10 minutes of rest.

Results are reported in the figure legends to FIG. 28-30.

1-123. (canceled)

124. A method of determining the status of warning system sensitivity (WSS)/defense reaction/reflex sensitivity (DRS) in a subject, including the steps of: measuring an applied stimulation at a threshold value of the stimulation in one or more sympathetic tone-neutral points and measuring an applied stimulation at the same threshold value in one or more sympathetic tone-dependent points, or measuring an applied stimulation at a threshold value in one or more sympathetic tone-dependent points and optionally comparing said threshold value to a predetermined or pre-established calibration threshold value.

125. A method according to claim 124, wherein the threshold value of the stimulation is the stimulation's nociception threshold value.

126. A method according to claim 124, wherein the applied stimulation is provided by means of an applied mechanical stimulation, such as an applied compressive force and, an applied vibrating force, or wherein the applied stimulation is provided by means of an applied thermal stimulation, such as an applied heat or cold source, or wherein the applied stimulation is provided by means of an applied electrical stimulation, such as an alternating current or direct current, or wherein the applied stimulation is provided by means of an applied radiation, such as an applied infrared, visible and/or ultraviolet light or combined spectra thereof, or wherein.

127. A method according to claim 124, wherein the applied stimulation is provided by means of an applied chemical stimulation.

128. A method according to claim 127, wherein the applied chemical stimulation is provided by means of an applied organic or inorganic compound.

129. The method according to claim 124, wherein the applied stimulation in the sympathetic tone dependent point is direct stimulation of a at least one polymodal sensor cell and wherein the threshold value in the one or more sympathetic tone-dependent points is the firing threshold of said at least one polymodal sensor cell.

130. The method according to claim 129, wherein the direct stimulation is selected from AC stimulation, DC stimulation, stimulation with a neurotransmitter, and chemical stimulation.

131. A method according to claim 124, wherein the determination is performed by means of a system for measuring the applied stimulation.

132. A method according to claim 124, wherein the measuring of an applied stimulation at a threshold value of the stimulation in one or more sympathetic tone-neutral points is carried out anteriorly on the upper side of the clavicle and/or posteriorly on the spinal column corresponding to TH 10-11 and/or on a finger and/or on a toe.

133. A method according to claim 124, wherein the measuring of an applied stimulation at a threshold value of the stimulation is carried out in one or more sympathetic tone-dependent points at one or more locations on or in the skin which innervationally correspond(s) to the nerve supply to the heart of the sympathetic nervous system.

134. A method according to claim 124, wherein the measuring of an applied stimulation at a threshold value of the stimliation in one or more sympathetic tone-dependent points is carried out in one or more of the points: C.V. 17 in the middle of the sternum and/or St 18 between two ribs below the nipple and/or Per 1 between the nipple and the anterior axillary fold and/or on the spinal column corresponding to TH 3-6, where the most sore point of the said points are chosen.

135. A method of quantitative and/or qualitative determination of status of WSS/DRS in an animal, including a human being, said method including:
a) storage of a calibration threshold value and a stimulation threshold value, the calibration threshold value being a quantitative measure of a nociception threshold value in a sympathetic tone-neutral point in or on the animal's body and the stimulation threshold value being a quantitative measure of a nociception threshold value in a sympathetic tone-dependent point in or on the animal's body, and subsequently;
b) calculation of an indication value of WSS and/or DRS and/or sympathetic tone and/or level of stress by comparing the stimulation threshold value with the calibration threshold value, whereby the indication value of WSS and/or DRS and/or sympathetic tone and/or level of stress is a measure of the warning system sensitivity or and/or sympathetic tone and/or level of stress in the human being.

136. A method of quantitative and/or qualitative determination of status of WSS/DRS and/or sympathetic tone and/or level of stress in a animal, including a human being, said method including:
a) storage of a calibration threshold value and a stimuilation threshold value, the calibration threshold value being a quantitative measure of polymodal sensor cell firing threshold in a sympathetic tone-neutral point in or on the animal's body and the stimulation threshold value being a quantitative measure of polymodal sensor cell firing threshold in a sympathetic tone-dependent point in or on the animal's body, and subsequently;
b) calculation of an indication value of warning system sensitivity by comparing the stimulation threshold value with the calibration threshold value, whereby the indication value of warning system sensitivity and/or sympathetic tone and/or level of stress is a measure of the warning system sensitivity and/or sympathetic tone and/or warning system sensitivity in the animal.

137. A method according to claim 134, wherein the calibration threshold value is a pre-determined or pre-established constant value which is stored permanently.

138. A method according to claim 134, wherein the calibration threshold value is zero, whereby the indication value is identical to the stimulation threshold value or is a function of the stimulation threshold value.

139. A method according to claim 134, wherein the calibration threshold value and the stimulation threshold value are measured substantially simultaneously.

140. A method according to claim 139, wherein the indication value of the sympathetic tone is a mathematical combination of the calibration threshold value and the stimulation threshold value.

141. A method according to claim 139, wherein the indication value is a function, such as a monotonic function, of the ratio between the calibration threshold value and the stimulation threshold value.

142. A method according to claim 139, wherein the indication value is a function of the difference between the calibration threshold value and the stimulation threshold value.

143. A method for controlling the progress of a patient's therapeutic regimen, wherein the efficacy and/or patient compliance of said regimen is dependent on sympathetic tone and/or stress level and/or level of WSS and/or DRS in said patient, comprising
i) determining one or more times during the course of the therapeutic regimen the sympathetic tone and/or level of stress in said patient, and
ii) adjusting the therapeutic regimen based on an integrated measure of the patient's benefit from the therapeutic regimen and the sympathetic tone and/or level of stress and/or level of WSS and/or DRS determination is step i.

144. A method according to claim 143, wherein the determination in step i is performed according to the method.

145. A method according to claim 143, wherein the therapeutic regimen is selected from the group consisting of treatment with SSRI (Selective Serotonine Re-uptake Inhibitors), psychopharmalogical treatment of psychological, mental or behavorial disturbances, which are influenced by stress, including depression, other mood disorders, addiction, dependence disorder, neurosis, and suicidal behavorior, Insulin-treatment in diabetes, nicotine substitution used as adjuvant therapy in smoking cessation, hormonal therapy in postmenopausal syndromes, hormone or other therapeutic means with respect to reproduction, fertility and miscarriage treatment, antiinflammatory therapy in acute and chronic inflammation, antiinfective therapy in infectious diseases, treatment of hypo- or hyperthyroid conditions, treatments with respect to dental care, treatment of diseases in heart, vessels, and kidney using cardiovascular drugs, treatment of ulcers, irritable bowel syndrome, malabsorption, nausea, and other symptoms using gastrointestinal drugs, pharmacological treatment with body weight lowering drugs, exercise programmes, relaxation programmes, diet programmes, counselling or coaching, stress-management programs, personal development programmes, personal performance programmes and self-care programmes.

146. A method for prevention or reduction of undesired or unproductive stress or reducing WSS/DRS in a patient, the method comprising
a) determination of sympathetic tone and/or the stress level and/or level of WSS and/or DRS in the patient according to the method of claim 124, and
b) if the determination in step a indicates an elevated sympathetic tone and/or level of stress and/or increased level of WSS and/or DRS, subjecting a sympathetic tone dependent point to a stimulation having a lower intensity than the stimulation threshold value for a period of time.

147. A method according to claim 146, further comprising, after step b,
c) determination of sympathetic tone and/or the stress level and/or level of WSS and/or DRS in the patient, and if the determination does not indicate a less elevated sympathetic tone and/or level of stress and/or level of WSS and/or DRS, subjecting a sympathetic tone dependent point to a stimulation having a lower intensity than the stimulation threshold value for a period of time which is different from the period of time in step a, and
d) repeating step c until the determination indicates a less elevated sympathetic tone and/or level of stress or level of WSS and/or DRS than the determination in step a.

148. A method of claim 146, wherein subjection of the sympathetic tone dependent point to the lower stimulation intensity is controlled by indicating a correct stimulation intensity by means of a visible or audible indication.

149. A method for prognosis or prophylaxis of a disease in a patient, comprising
1) determining the status of WSS and/or DRS and/or sympathetic tone and/or level of stress in the patient, and subsequently
2) providing a prognosis for the patient with respect to the disease by incorporating in the determination of the prognosis the result of the determination in step 1, a determination in step 1 indicating a low sympathetic tone and/or level of stress and/or level of WSS and/or DRS being indicative of a better prognosis than a determination in step 1 of a higher sympathetic tone and/or level of stress and/or level of WSS and/or DRS. 35. A method according to claim 149, wherein the determination in step 1 is performed.

150. A method according to claim 149, wherein the disease is selected from the group consisting of
an acute, subacute or chronic inflammatory condition;
a condition in which immunological reactions cause harm to human organs or in which insufficient function of the immunological system cause impaired function and/or disease;
an acute, subacute and chronic infectious disease;
a cardiovascular disturbance, which is affected by sympathetic tone, such as circulatory shock, atheriosclerosis, thrombosis, an ischemic condition, infarction, cardiac arrhythmia, hypertension;
a neoplastic growth disturbance;
an aquired metabolic disturbance;
a poisining or physical damage due to mechanic, thermal, electrical or radiation energy;
a psychological, mental or behavioural disturbance, which are influenced by sympathetic tone, such as depression or other mood disorders, an addiction or dependence disorder of any kind, a neurosis, a suicidal behaviour,
a sleep disturbance, fatigue, a stress-related complain of psychological and/or mental character;
a fertility decrease in both female and male;
a gynaecological disturbance, which is influenced by sympathetic tone, such as premenstrual syndrome, dysmenorhea, menopause problems, hyperemesis gravidarum, preeclampsia and eclampsia, premature labor, situs invertus, induction of Labor, postpartum hemorhage;
an otolaryngological disturbance, which is influenced by sympathetic tone, such as tinnitus and presbyacusis;
a dermatological disturbance, which is influenced by sympathetic tone such as pruritus;
a gastrointestinal disease with stress-sensitive clinical signs and symptoms, such as gastric and duodenal ulcer, irritable bowel syndrome, malabsorption, diarrhea, constipation, nausea, and vomiting;
a neurological disturbance, such as tension headache, migraine, concussion, Parkinson's disease, Alzheimer's disease, intracranial traumas, and neuropathies an endocrinological disorder, such as diabetes, hypothyroidism, hyperthyroidism, an adrenocortical disorder, adrenomedullary disorder, a hypothamic disorders, a pituitary disorder, and polycystic ovary syndrome;
a allergy, such as one with reactions in skin, bronchi, and the gastrointestinal tract;
a pulmonary diseases with impaired gas exchange, such as bronchitis and emphysema;
a disease in joints and bone, such as, acute or chronic arthritis and osteoporosis;
a disorder related to changes in body weight composition, such as obesity, weight loss, cachexia;
a sodium and water-retaining disease state, such as heart failure, kidney failure, liver failure; and pain.

151. A system for measuring the sympathetic tone and/or level of stress and/or the status of WSS/DRS in an animal, including a human being, said system including:
a) memory means for storing a nociception calibration threshold value determined in a sympathetic tone-neutral point on or in the animal's body and for storing a nociception stimulation threshold value determined in a sympathetic tone-dependent point on or in the animal's body;
b) an electronic circuit programmed to data process the nociception calibration threshold value and the nociception stimulation threshold value so as to obtain the measurement; and
c) user-operated means for applying a discomfort-evoking stimulus to the animal's body and user-operated storage means adapted to store the nociception stimulation threshold value resulting from a first user operation;
d) user-operated means for applying a discomfort-evoking stimulus to the animal's body and user-operated storage means adapted to store the nociception stimulation threshold value resulting from a second user operation;
wherein the discomfort-evoking stimulus involves 1) vibration applied by means of a first vibration base and/or 2) heat applied by means of a first heating base and/or 3) electricity applied by means of a first electricity base, and wherein the means for applying a discomfort-evoking stimulation is/are adapted to apply a stimulus which is gradually increased, the storage means being adapted to store a stimulation level at a moment in time corresponding to the first and the second user operation, respectively.

152. A system according to claim 151, wherein the calibration threshold value is a pre-determined or pre-established constant value which is stored permanently.

153. A system according to claim 151, wherein the calibration threshold value is zero.

154. A system according to claim 152, wherein the means for applying a discomfort-evoking stimulus is contained in a first unit and where the said electronic circuit is contained in a second unit.

155. A system according to claim 154, wherein the first and the second units are adapted to allow wireless communication between the first unit and the second units.

156. A system according to claim 152, wherein the means for applying a discomfort-evoking stimulus and the said electronic circuit are integrated in one and the same apparatus.

157. A system according to claim 151, wherein the vibration base comprises an essentially spherical or hemispherical vibration head.

158. A system according to claim 157, wherein the diameter of the vibration head is less than 1 cm, such as between 0.05 mm and 0.5 mm, or less than 0.5 mm, such as between 1 mm and 4 mm.

159. A system according to claim 157, further comprising a magnetic actuator.

160. A system according to claim 159, wherein the actuator is arranged along an essentially linear axis.

161. A system according to claim 160, wherein the actuator comprises a sliding bar and a plurality of sliding elements arranged along the sliding bar.

162. A system according to claim 161, wherein the sliding elements define at least one circumferential groove for supporting windings wound around the sliding elements.

163. A system according to claim 159, further comprising a plurality of magnets and separators.

164. A system according to claim 163, wherein the sliding bar defines an internal, longitudinally extending cavity or bore, and wherein the magnets and separators are alternatingly positioned in said sliding cavity or bore.

165. A system according to claim 161, wherein the sliding bar is coated with a friction reducing material.

166. A system according to claim 163, wherein the magnets are arranged to create a magnetic field essentially perpendicular to the sliding bar.

167. A system according to claim 162, further comprising a control unit enabling alternation of a current flowing in the windings.

168. A system according to claim 167, wherein the magnetic actuator is arranged to create vibrations by alternating the current flowing in the windings.

169. A system for measuring the sympathetic tone and/or the level of stress and/or the status of the WSS/DRS in an animal, including a human being, said system including a vibration base with a vibration head adapted to exert an outer vibrating force on the animal's body, a sensor for measuring the vibrating force exerted by the vibration base on the body, an electronic circuit adapted to store a first measured vibrating force and a second measured vibrating force, respectively, and to calculate a read-out value as an expression of the ratio between the first measured vibrating force and the second measured vibrating force.

170. A system according to claim 169, further comprising a read-out unit for displaying the read-out value.

171. A system according to claim 169, wherein the vibration base and the sensor are integrated in a first unit and wherein the said electronic circuit is integrated in a second unit.

172. A system according to claim 171, wherein the first and the second units are adapted such to allow wireless communication between the first unit and the second unit.

173. A system according to claim 169, wherein the vibration base, the sensor and the said electronic circuit are integrated in one and the same apparatus.

174. A system according to claim 169, wherein the vibration head of the vibration base is essentially spherical or hemispherical.

175. A system according to claim 174, wherein the diameter of the vibration head is less than 1 cm, preferably between 0.05 mm and 0.5 mm.

176. A system according to claim 169, wherein the sensor comprises a piezoresistive force sensor.

177. A system according to claim 169, said system being hand-held and supplied with power by one or more batteries.

178. A system according to claim 170, wherein the read-out unit comprises an electronic display.

179. A system according claim 170, wherein the electronic circuit is adapted to determine the read-out value as one of a number, e.g. four, discrete read-out values (0, 1, 2, 3), the ratio between the first measured value and the second measured value being allocated a discrete read-out value (0, 1, 3, 4) displayed on the read-out unit.

180. A system according to claim 179, wherein the discrete read-out value (0, 1, 2, 3) is non-proportional to the ratio between the first measured value and the second measured value.

181. A system according to claim 169, wherein the electronic circuit is adapted to calculate the first measured value as an average of a number of measured values and calculate the second measured value as an average of a number of measured values.

182. A system according to claim 151, wherein the area of a contact face of the heating base is less than 4 cm², such as between 1 and 2 cm², or less than 1 cm², such as between 0.5 and 1 cm².

183. A system according to claim 182, further comprising a control unit, the control unit being provided to change the temperature of the contact face.

184. A system according to claim 183, wherein the control unit increases the temperature of the contact face until a build-in maximum temperature is reached or until a user interrupts the upward temperature movement.

185. A system according to claim 183, wherein the contact face is cooled by natural cooling.

186. A system for measuring the sympathetic tone and/or level of stress and/or status of WSS/DRS in an animal, including a human being, said system including a heating base with a contact face adapted to apply heat on the animal's body, a sensor for measuring the temperature applied by the heating base on the body, an electronic circuit adapted to store a first measured temperature and a second measured temperature, respectively, and to calculate a read-out value as an expression of the ratio between the first measured temperature and the second measured temperature.

187. A system according to claim 186, further comprising a read-out unit for displaying the read-out value.

188. A system according to claim 186, wherein the heating base and the sensor are integrated in a first unit and wherein the said electronic circuit is integrated in a second unit.

189. A system according to claim 188, wherein the first and the second units are adapted such to allow wireless communication between the first unit and the second unit.

190. A system according to claim 186, wherein the heating base, the sensor and the said electronic circuit are integrated in one and the same apparatus.

191. A system according to claim 186, wherein the area of a contact face of the heating base is less than 4 cm², preferably between 1 and 2 cm².

192. A system according to claim 186, wherein the sensor comprises a thermoresistive sensor.

193. A system according to claim 186, said system being hand-held and supplied with power by one or more batteries.

194. A system according to claim 187, wherein the read-out unit comprises an electronic display.

195. A system according to claim 187, wherein the electronic circuit is adapted to determine the read-out value as one of a number, e.g. four, discrete read-out values (0, 1, 2, 3), the ratio between the first measured value and the second measured value being allocated a discrete read-out value (0, 1, 3, 4) displayed on the read-out unit.

196. A system according to claim 195, wherein the discrete read-out value (0, 1, 2, 3) is non-proportional to the ratio between the first measured value and the second measured value.

197. A system according to claim 186, wherein the electronic circuit is adapted to calculate the first measured value as an average of a number of measured values and calculate the second measured value as an average of a number of measured values.

198. A system according to claim 151, wherein the electricity base comprises an essentially spherical or hemispherical electricity head.

199. A system according to claim 198, wherein the diameter of the electricity head is less than 1 cm, such as between 0.05 mm and 0.5 mm, or less than 0.5 mm, such as between 1 mm and 4 mm.

200. A system according to claim 198, further comprising an electric actuator.

201. A system according to claim 200, further comprising a control unit enabling current flow in the electricity head.

202. A system according to claim 201, wherein the control unit further enables changing of current from alternating current to direct current and vice versa.

203. A system according to claim 202, wherein the control unit is arranged to increase a direct current flow in the electricity head until a build-in maximum current flow is reached or until a user interrupts the increasing current flow.

204. A system according to claim 202, wherein the control unit is arranged to increase a frequency of an alternating current flow in the electricity head until a build-in maximum frequency is reached or until a user interrupts the increasing frequency.

205. A system for measuring the sympathetic tone and/or level of stress and/or level of WSS and/or DRS in an animal, including a human being, said system including an electricity base with an electricity head adapted to exert an outer electric force on the animal's body, a sensor for measuring the electric force exerted by the electricity base on the body, an electronic circuit adapted to store a first measured electric force and a second measured electric force, respectively, and to calculate a read-out value as an expression of the ratio between the first measured electric force and the second measured electric force.

206. A system according to claim 205, wherein the system includes a read-out unit for displaying the read-out value.

207. A system according to claim 205, wherein the electricity base and the sensor are integrated in a first unit and wherein the said electronic circuit is integrated in a second unit.

208. A system according to claim 207, wherein the first and the second units are adapted such to allow wireless communication between the first unit and the second unit.

209. A system according to claim 205, wherein the electricity base, the sensor and the said electronic circuit are integrated in one and the same apparatus.

210. A system according to claim 205, wherein the electricity head of the electricity base is essentially spherical or hemispherical.

211. A system according to claim 210, wherein the diameter of the electricity head is less than 1 cm, preferably between 0.05 mm and 0.5 mm.

212. A system according to claim 205, wherein the sensor comprises an electric sensor.

213. A system according to claim 205, said system being hand-held and supplied with power by one or more batteries.

214. A system according to claim 206, wherein the read-out unit comprises an electronic display.

215. A system according to claim 206, wherein the electronic circuit is adapted to determine the read-out value as one of a number, e.g. four, discrete read-out values (0, 1, 2, 3), the ratio between the first measured value and the second measured value being allocated a discrete read-out value (0, 1, 3, 4) displayed on the read-out unit.

216. A system according to claim 215, wherein the discrete read-out value (0, 1, 2, 3) is non-proportional to the ratio between the first measured value and the second measured value.

217. A system according to claim 205, wherein the electronic circuit is adapted to calculate the first measured value as an average of a number of measured values and calculate the second measured value as an average of a number of measured values.

218. Use of measurement(s) of increased nociception (hyperalgesia) for determining the the status of WSS/DRS in a subject.

219. Use of measurement(s) of polymodal sensor cell firing threshold for determining the the status of warning system sensitivity in a subject.

220. A method for determining myocardial oxygen consumption in a subject, the method comprising determining sympathetic tone and/or level of stress and/or status of warning system sensitivity according to the method of claim 124 in the subject and determining the level of myocardial oxygen consumption based on a predetermined correlation to measurements or sympathetic tone and/or level of stress and/or status of warning system sensitivity.

221. A method according to claim 220, wherein the correlation is based on a pre-established correlation between sympathetic tone and/or level of stress and/or WSS and/or DRS determinations and PRP (Pressure Rate Product) determinations.

222. A method for determining cardiac work capacity in a subject, the method comprising a determination according to the method of claim 124 under stress conditions and during rest, respectively, and determining the cardiac work capacity of the subject based on a mathematical combination of determinations under stress conditions and during rest.

223. A method according to claim 222, wherein the mathematical combination is selected from a difference, a ratio, and any monotonic function thereof.

224. A method for indirectly determining the level of glycated haemoglobin in a diabetic subject, the method comprising a determination according to the method of claim 124 and determining the glycated haemoglobin level as a monotonic function of said determination.

225. A method according to claim 224, wherein it has been pre-established that the subject does not suffer from a high level of chronic stress or has an increase in level of WSS and/or DRS.

226. The method for preventing/reducing stress according to claim 146 in a diabetic subject, comprising consolidating the measurements of stimulation threshold values with at least one measurement of glycated haemoglobin

227. The method according to claim 226, wherein a measurement indicating an elevated level of glycated haemoglobin indicates that the subject is chronically stressed at a high level and/or has an elevated level of WSS and/or DRS.

228. A method according to claim 124 for monitoring the level and/or nature of stress and/or the level of WSS and/or DRS in a subject who is undergoing an investigative trial of a potentially stress-relevant nature or of a potentially warning system sensitive nature.

229. A method for determining the status of warning system sensitivity (WSS) in a subject, comprising determining an applied stimulation at a threshold value in point(s) on or in the body where nociception is dependent on sympathetic tone and correlating the stimulation threshold with a WSS value.

230. The method according to claim 229, wherein the subject is in an environment substantially free from acute stress-inducing factors or other factors which could effect an increase an increased sympathetic tone.

231. The method according to claim 229, wherein the measurement of the applied stimulation is performed.

232. A method for modulating the status of warning system sensitivity, the method comprising a) determination of sympathetic tone and/or the stress level and/or WSS in a patient according to claim 124, and if the determination indicates an elevated sympathetic tone and/or level of stress and/or WSS, subjecting a sympathetic tone dependent point to a stimulation having a lower intensity than the stimulation threshold value for a period of time.

233. The method according to claim 232, wherein the stimulation having a lower intensity is selected from acupressure, acupuncture therapy, and direct stimulation of polymodal sensor cells.

234. A method for determining whether the level of stress and/or the level of sympathetic tone and/or WSS is elevated in an animal, such as a human being, the method comprising determining the presence in the animal of a noxious withdrawal reflex (NWR) in response to a stimulus, said stimulus being one which does not elicit an NWR in a majority of individual animals in a population having a normal sympathetic tone or not being stressed or not having increased level of WSS and/or DRS but which does elicit an NWR in a majority of individual animals in a population having increased sympathetic tone or high level of stress or increased level of WSS and/or DRS,
where the observation of an NWR in response to said stimulus is an indication that said animal has an increased sympathetic tone and/or level of stress and/or level of WSS and/or DRS.

235. A method for reducing the level of sympathetic tone and/or the level of stress and/or pain perception and/or level of WSS and/or DRS in a subject in need thereof, the method comprising
a) stimulating polymodal sensor cell(s) in the subject so as to gauge the firing threshold, where the stimulation preferably is by means of electrical stimuli;
b) monitoring afferent impulses originating from the polymodal sensor cell(s) in response to the stimuation in step a);
c) if the monitoring in step b) reveals a lowered threshold for firing, which is indicative of elevated psychological stress and/or level of WSS and/or DRS, stimulating efferent nerve fibres to change the physiological state of the subject, and d) repeating steps a)-c).

236. The method according to claim 235, wherein stimulating efferent nerve fibres in step c) involves epidural spinal cord stimulation.

237. A closed loop device for controlled stimulation of efferent nerve fibres, comprising
means for applying electrical stimuli to polymodal sensor cells,
means for measuring afferent impulses originating from the polymodal sensor cells or from efferent motor nerve cells;
means for comparing measured afferent impulses with standard values;
means for applying electrical stimuli to efferent nerve fibres; and
means for controlling the application of electrical stimuli to efferent nerve fibres as a response to the comparison between the afferent impulses with the standard values.
